(19) United States
(12) Patent Application Publication
(10) Pub. No.: US 2026/0049336 A1
(43) Pub. Date: Feb. 19, 2026

(54) BICISTRONIC AAV VECTORS ENCODING HEXOSAMINIDASE ALPHA AND BETA-SUBUNITS AND USES THEREOF

(71) Applicant: University of Massachusetts, Westborough, MA (US)

(72) Inventors: Miguel Sena Esteves, Westford, MA (US); Diane Golebiowski, Billerica, MA (US)

(21) Appl. No.: 19/374,990

(22) Filed: Oct. 30, 2025

Related U.S. Application Data
(62) Division of application No. 17/046,756, now Pat. No. 12,486,518.
(60) Provisional application No. 62/657,243, filed on Apr. 13, 2018.

(51) Int. Cl. C12N 15/86; A61K 48/00; A61P 25/00; C12N 9/24

(52) U.S. Cl. CPC C12N 15/86; A61P 25/00; C12N 9/2402; C12Y 302/01052; A61K 48/00; C12N 2750/14143; C12N 2830/50

(57) ABSTRACT

Aspects of the disclosure relate to bicistronic AAV nucleic acid constructs comprising a transgene encoding hexosaminidase A (HEXA) and hexosaminidase (HEXB) proteins. In some embodiments, the disclosure provides methods for treating or preventing lysosomal storage disorders, such as Tay-Sachs disease and Sandhoff disease, using bicistronic nucleic acid constructs described by the disclosure.

### RELATED APPLICATIONS

This application is a divisional of U.S. patent application Ser. No. 17/046,756, filed Oct. 9, 2020, which is a National Stage Application under 35 U.S.C. § 371 and claims the benefit of International Application No. PCT/US2019/027271, filed Apr. 12, 2019, which application claims the benefit under 35 U.S.C. § 119(e) of the filing date of U.S. Provisional Application Ser. No. 62/657,243, filed Apr. 13, 2018. The disclosure of each of the foregoing applications is incorporated herein by reference.

### FEDERALLY SPONSORED RESEARCH

This invention was made with government support under grant number NS093941, awarded by the National Institutes of Health. The government has certain rights in the invention.

### SEQUENCE LISTING

This application contains a sequence listing that has been submitted electronically as an XML file named “07917-0419002_SL_ST26.xml.” The XML file, created on Oct. 28, 2025, is 66,807 bytes in size. The material in the XML file is hereby incorporated by reference in its entirety.

### BACKGROUND OF INVENTION

Tay-Sachs disease (TSD) and Sandhoff disease (SD) are autosomal recessive lysosomal storage diseases (LSDs) caused by deficiency in hexosaminidase A (HexA) resulting from mutations in the HEXA or HEXB genes, respectively. HexA deficiency leads to storage of GM2 ganglioside in the central nervous system (CNS) and progressively deteriorating neurological function, developmental regression and eventually premature death. Currently there are no treatments available for these diseases collectively known as GM2 gangliosidoses.

### SUMMARY OF INVENTION

Aspects of the disclosure relate to recombinant AAV vectors for gene delivery. The disclosure is based, in part, on the discovery that a single AAV vector encoding simultaneously hexosaminidase alpha and beta-subunits allows less invasive and potentially more effective expression of HexA in the CNS of a subject (e.g., a subject having Tay-Sachs disease or Sandhoff disease). As described in the Examples section, compositions of the disclosure comprise two HexA subunits (e.g., encode HEXA and HEXB transcripts) in a configuration that has been shown to be functional in vivo and achieve important therapeutic milestones.

Accordingly, in some aspects, the disclosure provides an isolated nucleic acid construct comprising: a first expression cassette, comprising a nucleic acid encoding a hexosaminidase alpha-subunit (HexA) under the control of a first promoter, and a first intron; and, a second expression cassette, comprising a nucleic acid encoding a hexosaminidase beta-subunit (HexB) under the control of a second promoter, and a second intron. In some embodiments, the first expression cassette and the second expression cassette are flanked by adeno-associated virus (AAV) inverted terminal repeats (ITRs).

In some embodiments, the HexA comprises an amino acid sequence as set forth in SEQ ID NO: 1. In some embodiments, the HexB comprises an amino acid sequence as set forth in SEQ ID NO: 2.

In some embodiments, the first intron is positioned between the first promoter and the nucleic acid sequence encoding the HexA. In some embodiments, the first intron is a chimeric intron. In some embodiments, the first promoter is positioned proximal to an AAV ITR, optionally wherein the first promoter is positioned between an AAV ITR and a nucleic acid sequence encoding HexA. In some embodiments, the first promoter and/or the second promoter is a P2 promoter. In some embodiments, the second intron is positioned between the second promoter and the nucleic acid sequence encoding the HexB. In some embodiments, the second intron is a chimeric intron. In some embodiments, the second promoter is positioned proximal to an AAV ITR. In some embodiments, wherein the second promoter is positioned between an AAV ITR and a nucleic acid sequence encoding HexB.

In some embodiments, the first expression cassette comprises a first poly A signal (e.g., poly A tail) operably linked to the nucleic acid sequence encoding HexA, optionally wherein the first poly A signal (e.g., poly A tail) is a BGH poly A signal (e.g., BGH poly A tail). In some embodiments, the second expression cassette comprises a second poly A signal (e.g., poly A tail) operably linked to the nucleic acid sequence encoding HexB. In some embodiments, the second poly A signal (e.g., poly A tail) is an SV40 poly A signal (e.g., SV40 poly A tail). In some embodiments, the first poly A signal (e.g., poly A tail) and the second poly A signal (e.g., poly A tail) are positioned adjacent to one another. In some embodiments, the first expression cassette and the second expression cassette are orientated in opposing directions (e.g., the first expression cassette and the second expression cassette are transcribed toward one another).

In some aspects, the disclosure provides an isolated nucleic acid construct comprising: a first expression cassette, comprising a nucleic acid sequence encoding a hexosaminidase alpha-subunit; and, a second expression cassette, comprising a nucleic acid sequence encoding a hexosaminidase beta-subunit, wherein the first expression cassette and the second expression cassette are operably linked by a bidirectional promoter. In some embodiments, the first expression cassette and the second expression cassette are flanked by adeno-associated virus (AAV) inverted terminal repeats (ITRs).

In some embodiments, the HexA comprises an amino acid sequence as set forth in SEQ ID NO: 1. In some embodiments, the HexB comprises an amino acid sequence as set forth in SEQ ID NO: 2.

In some embodiments, the bidirectional promoter comprises at least one chicken beta-actin (CBA) promoter. In some embodiments, the bidirectional promoter comprises two CBA promoters, wherein the CBA promoters initiate transcription of the first expression cassette and the second expression cassette in opposite directions (e.g., transcription of the first expression cassette occurs in a direction that is distal with respect to the second expression cassette). In some embodiments, the bidirectional promoter comprises a CMV enhancer sequence. In some embodiments, the CMV enhancer sequence is positioned between the two CBA promoters.

In some embodiments, the first expression construct comprises a first poly A signal (e.g., first poly A tail), optionally wherein the first poly A signal (e.g., first poly A tail) is proximal to an AAV ITR. In some embodiments, the first expression construct comprises a second poly A signal (e.g., second poly A tail). In some embodiments, the second poly A signal (e.g., second poly A tail) is proximal to an AAV ITR. In some embodiments, the first and/or second poly A signal (e.g., poly A tail) is each selected from SV40 poly A signal (e.g., SV40 poly A tail), rabbit beta-globulin (RBG) poly A signal (e.g., RBG poly A tail), and bovine growth hormone (BGH) poly A signal (e.g., BGH poly A tail).

In some aspects, the disclosure provides an isolated nucleic acid comprising the sequence set forth in SEQ ID NO: 3-9. In some embodiments, an isolated nucleic acid encoding HexA protein and/or HexB is codon-optimized.

In some aspects, the disclosure provides a recombinant AAV (rAAV) comprising: a capsid protein; and an isolated nucleic acid sequence of any of the above. In some embodiments, the capsid protein is of a serotype selected from AAV1, AAV2, AAV3, AAV4, AAV5, AAV6, AAV7, AAV8, AAVrh8, AAV9, AAV10, AAVrh10, AAV-PHP.B, and AAV-PHP.eB.

In some embodiments, the isolated nucleic acid comprises an ITR selected from the group consisting of AAV1 ITR, AAV2 ITR, AAV3 ITR, AAV4 ITR, AAV5 ITR, or AAV6 ITR.

In some embodiments, the disclosure provides a host cell comprising the nucleic acid or the rAAV. In some embodiments, the host cell is a mammalian cell, yeast cell, bacterial cell, insect cell, plant cell, or fungal cell.

In some aspects, the disclosure provides a method for treating a lysosomal storage disease, the method comprising administering the isolated nucleic acid or the rAAV to a subject having or suspected of having a lysosomal storage disease. In some embodiments, a subject is a human.

In some embodiments, the lysosomal storage disease is Tay-Sachs disease or Sandhoff disease. In some embodiments, the subject is characterized as having a mutation in a HEXA gene resulting in reduced (or loss of) function of a hexosaminidase alpha-subunit of the subject. In some embodiments, the subject is characterized as having a mutation in a HEXB gene resulting in reduced or loss of function of a hexosaminidase beta-subunit of the subject.

In some embodiments, the rAAV is administered by intracranial injection, intracerebral injection, or injection into the CSF via the cerebral ventricular system, cisterna magna, or intrathecal space. In some embodiments, the subject is administered the isolated nucleic acid or the rAAV during a pre-symptomatic stage of the lysosomal storage disease. In some embodiments, the pre-symptomatic stage of the lysosomal storage disease occurs between birth (e.g., perinatal) and 4-weeks of age.

### BRIEF DESCRIPTION OF DRAWINGS

FIG. 1 shows a schematic depiction of the structure of bicistronic AAV vectors encoding simultaneously hexosaminidase alpha and beta-subunits. In some embodiments, the AAV-BiP2i-HexAB vector carries two copies of a small promoter with a small intron (P2i) at the ends of the AAV genome driving expression of Hex alpha and beta subunits in opposing directions. In some embodiments, the AAV-BiCBA-HexAB vector carries a bidirectional CBA promoter designed by duplication of the chicken beta-actin promoter in opposing directions with a CMV enhancer in the middle.

FIGS. 2A-2C show the behavioral performance of AAV-treated Sandhoff (SD) mice (Hexb^{-/-}) remained stable over time. Motor coordination and performance of AAV-treated SD mice and controls were assessed at 60, 90, 105, 120, and 149 days of age. FIG. 2A shows the performance of SD mice using the accelerating rotarod test (4-40 rpm over 300 seconds). FIG. 2B shows the performance of SD mice using the inverted screen test assessing latency to falling and FIG. 2C shows the performance of SD mice using the inverted screen test assessing the number of hindlimb movements.

FIG. 3 shows that systemic AAV treatment reduces GM2 ganglioside content throughout the central nervous system (CNS) of SD mice. GM2 ganglioside content was measured at 150 days of age in AAV-treated SD mice (4×10¹² vg AAV9 or 1×10¹² vg AAV-PHP.B) using a liquid chromatography tandem mass spectrometry (LC-MS/MS) method. GM2 ganglioside levels in the CNS of normal animals was below the detection limit of the method.

FIGS. 4A-4D show increased hexosaminidase activity in brain of 150 day old SD mice treated with 4×10¹² vg AAV9 or 1×10¹² vg AAV-PHP.B vectors. FIG. 4A shows HexA enzyme activity in the cerebrum using the artificial substrate MUGS. FIG. 4B shows total Hex (HexA+HexB) activity in the cerebrum using the artificial substrate MUG. FIG. 4C shows HexA enzyme activity in the cerebellum using MUGS. FIG. 4D shows total Hex activity in the cerebellum using MUG.

FIG. 5 shows increased total hexosaminidase activity in the liver using the artificial substrate MUG upon systemic delivery of AAV-PHP.B-BiCBA-HexAB in SD mice.

FIGS. 6A-6B show systemic AAV treatment extends survival of SD mice. Four week-old SD mice were treated with either low dose or high dose AAV therapies and survival was assessed. FIG. 6A shows treatment with low dose AAV9-Bic (1×10¹² vg), AAV9-P2I (1×10¹² vg), AAV-PHP.B-Bic (3×10¹¹ vg), AAV-PHP.B-P2I (3×10¹¹ vg), vectors or PBS (KO). FIG. 6B shows treatment with high dose AAV9-Bic (4×10¹² vg), AAV9-P2I (4×10¹² vg), AAV-PHP.B (1×10¹² vg), AAV-PHP.B-Bic-P2I (1×10¹² vg), vectors or PBS (KO).

FIG. 7 shows systemic delivery of AAV-PHP.B-BiCBA-HexAB vector restores Hex activity throughout the brain of SD mice. Distribution of Hex enzyme activity was assessed at one month post-injection using an enzyme specific histochemical stain of sagittal brain sections. Sections of all three AAV-treated mice are shown.

FIGS. 8A-8D show data indicating that neonatal ICV treatment of GM2 mice with bicistronic AAV9 improves neurochemistry, behavior and survival. FIG. 8A shows GM2 ganglioside content in cerebrum [C], cerebellum and brainstem [Cb+BS] at 1 month of age after injection of AAV9-BiCB-HexAB. FIG. 8B shows assessment of motor performance by an accelerating rotarod (4-40 rpm over 5 min) test. FIG. 8C shows data relating to an inverted screen test (max of 120 sec) at 120 and 150 days of age. FIG. 8D shows Kaplan-Meier survival plot. Median survival of untreated GM2 mice is 129.5 days. Two-sided t-tests were used to compare outcomes between AAV and untreated GM2 controls (**** P<0.0001; *** P<0.001; ** P<0.01).

FIGS. 9A-9C show data indicating lumbar intrathecal delivery of AAV9-BiCB-HexAB improves motor performance and increases survival of GM2 mice. FIG. 9A shows assessment of motor performance over time in GM2 mice treated with 1×1012 vg at ˜6 weeks of age using the accelerating rotarod (4-40 rpm over 5 min) test. FIG. 9B shows data relating to an inverted screen test (max of 120 sec). FIG. 9C shows a Kaplan-Meier analysis indicating a significant increase (P<0.0001) in survival of AAV treated GM2 mice with a current median survival of 265 days compared to 129.5 days for untreated GM2 mice. Log-rank test was used to assess survival benefit.

### DETAILED DESCRIPTION OF INVENTION

Adeno-associated virus (AAV) mediated gene therapy is one experimental approach for treatment of TSD and SD, for example via intracranial delivery of a transgene to brain parenchyma. Overexpression of both alpha and beta-subunits is necessary as HexA is a αβ heterodimer. This is usually achieved by co-injection of two vectors encoding Hex alpha and beta-subunits separately due to the transgene size limitation of recombinant AAV vectors (˜4.7 kb). Delivery of two separate vectors through the bloodstream or CSF compromises the efficacy of this approach as HexA overexpression requires co-infection of cells with both vectors, and the likelihood of this even declines precipitously as the vectors are diluted upon infusion into these body fluids.

In some aspects, the disclosure relates to compositions and methods useful in the treatment of lysosomal storage disorders, for example Tay-Sachs disease or Sandhoff disease. The disclosure is based, in part, on recombinant AAV vectors (e.g., isolated nucleic acids) and recombinant adeno-associated viruses (rAAVs) comprising expression cassettes configured for bicistronic expression of multiple (e.g., 2, 3, etc.) therapeutic transgenes in the CNS of a subject

### Isolated Nucleic Acids

In some aspects, the disclosure provides isolated nucleic acids (e.g., bicistronic expression constructs, such as rAAV vectors) that are useful in expressing therapeutic transgenes in the CNS of a subject.

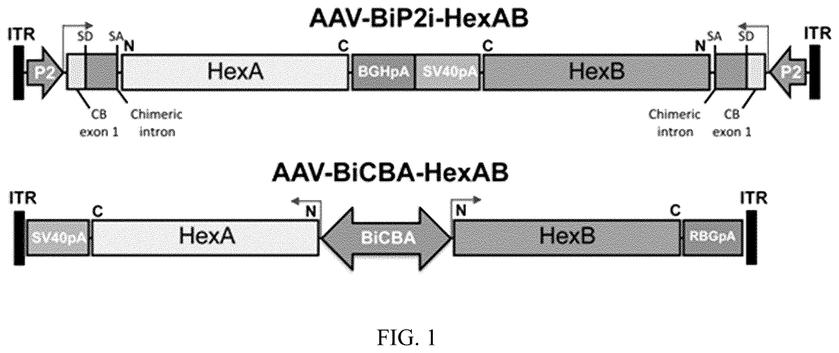

In some embodiments, a therapeutic transgene encodes one or more proteins associated with a lysosomal storage disease (e.g., Tay-Sachs disease, Sandhoff disease), for example HexA protein, HexB protein, or HexA and HexB protein. In some embodiments, a HexA protein comprises an amino acid sequence as set forth in SEQ ID NO: 1. In some embodiments, a HexB protein comprises an amino acid sequence as set forth in SEQ ID NO: 2. In some embodiments, a transgene (e.g., a transgene encoding HexA) encodes a sequence that is at least 70% (e.g., at least 70%, 80%, 90%, 95%, 99%, etc.) identical to an amino acid sequence as set forth in SEQ ID NO: 1. In some embodiments, a transgene (e.g., a transgene encoding HexB) encodes an amino acid sequence that is at least 70% (e.g., at least 70%, 80%, 90%, 95%, 99%, etc.) identical to an amino acid sequence as set forth in SEQ ID NO: 2. In some embodiments a transgene encodes a bicistronic expression construct harboring two expression cassettes in opposite orientations as shown in FIG. 1, AAV-BiP2i-HexAB and AAV-BiCBA-HexAB. Each of the HexA and HexB proteins encoded by a bicistronic construct may comprise an amino acid sequence that is at least 70% (e.g., at least 70%, 80%, 90%, 95%, 99%, etc.) identical to an amino acid sequence as set forth in SEQ ID NO: 1 or 2, respectively.

In some embodiments, a transgene (e.g., a transgene encoding HexA) comprises a nucleic acid sequence that is at least 70% (e.g., at least 70%, 80%, 90%, 95%, 99%, etc.) identical to a HexA-encoding nucleic acid sequence (e.g. wild-type HexA nucleic acid sequence, codon-optimized HexA encoding sequence, etc.) as set forth in any one of SEQ ID NOs: 3-9 (or a portion thereof). In some embodiments, a transgene (e.g., a transgene encoding HexB) comprises a nucleic acid sequence that is at least 70% (e.g., at least 70%, 80%, 90%, 95%, 99%, etc.) identical to a HexB-encoding nucleic acid sequence (e.g. wild-type HexB nucleic acid sequence, codon-optimized HexB encoding sequence, etc.) as set forth in any one of SEQ ID NOs: 3-9 (or a portion thereof).

A “nucleic acid” sequence refers to a DNA or RNA sequence. In some embodiments, proteins and nucleic acids of the disclosure are isolated. As used herein, the term “isolated” means artificially produced. As used herein with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. As used herein with respect to proteins or peptides, the term “isolated” refers to a protein or peptide that has been isolated from its natural environment or artificially produced (e.g., by chemical synthesis, by recombinant DNA technology, etc.).

The isolated nucleic acids of the invention may be recombinant adeno-associated virus (AAV) vectors (rAAV vectors). In some embodiments, an isolated nucleic acid as described by the disclosure comprises a region (e.g., a first region) comprising a first adeno-associated virus (AAV) inverted terminal repeat (ITR), or a variant thereof. The isolated nucleic acid (e.g., the recombinant AAV vector) may be packaged into a capsid protein and administered to a subject and/or delivered to a selected target cell. “Recombinant AAV (rAAV) vectors” are typically composed of, at a minimum, a transgene and its regulatory sequences, and 5′ and 3′ AAV inverted terminal repeats (ITRs). The transgene may comprise a region encoding, for example, a protein and/or an expression control sequence (e.g., a poly-A tail), as described elsewhere in the disclosure.

Generally, ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al., “Molecular Cloning. A Laboratory Manual”, 2d ed., Cold Spring Harbor Laboratory, New York (1989); and K. Fisher et al., J Virol., 70: 520 532 (1996)). An example of such a molecule employed in the present invention is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types. In some embodiments, the isolated nucleic acid (e.g., the rAAV vector) comprises at least one ITR having a serotype selected from AAV1, AAV2, AAV3, AAV4, AAV5, AAV6, and variants thereof.

In some embodiments, the isolated nucleic acid further comprises a region (e.g., a second region, a third region, a fourth region, etc.) comprising a second AAV ITR. In some embodiments, the second AAV ITR has a serotype selected from AAV1, AAV2, AAV3, AAV4, AAV5, AAV6, and variants thereof.

In addition to the major elements identified above for the recombinant AAV vector, the vector also includes conventional control elements which are operably linked with elements of the transgene in a manner that permits its transcription, translation and/or expression in a cell transfected with the vector or infected with the virus produced by the invention. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (e.g., Kozak consensus sequence); sequences that enhance protein stability; and when desired, sequences that enhance secretion of the encoded product. A number of expression control sequences, including promoters which are native, constitutive, inducible and/or tissue-specific, are known in the art and may be utilized.

As used herein, a nucleic acid sequence (e.g., coding sequence) and regulatory sequences are said to be operably linked when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein or polypeptide. Similarly two or more coding regions are operably linked when they are linked in such a way that their transcription from a common promoter results in the expression of two or more proteins having been translated in frame. In some embodiments, operably linked coding sequences yield a fusion protein.

A region comprising a transgene (e.g., a second region, third region, fourth region, etc.) may be positioned at any suitable location of the isolated nucleic acid. The region may be positioned in any untranslated portion of the nucleic acid, including, for example, an intron, a 5′ or 3′ untranslated region, etc.

In some cases, it may be desirable to position the region (e.g., the second region, third region, fourth region, etc.) upstream of the first codon of a nucleic acid sequence encoding a protein (e.g., a protein coding sequence). For example, the region may be positioned between the first codon of a protein coding sequence) and 2000 nucleotides upstream of the first codon. The region may be positioned between the first codon of a protein coding sequence and 1000 nucleotides upstream of the first codon. The region may be positioned between the first codon of a protein coding sequence and 500 nucleotides upstream of the first codon. The region may be positioned between the first codon of a protein coding sequence and 250 nucleotides upstream of the first codon. The region may be positioned between the first codon of a protein coding sequence and 150 nucleotides upstream of the first codon.

In some cases (e.g., when a transgene lacks a protein coding sequence), it may be desirable to position the region (e.g., the second region, third region, fourth region, etc.) upstream of the poly-A signal of a transgene. For example, the region may be positioned between the first base of the poly-A signal and 2000 nucleotides upstream of the first base. The region may be positioned between the first base of the poly-A signal and 1000 nucleotides upstream of the first base. The region may be positioned between the first base of the poly-A signal and 500 nucleotides upstream of the first base. The region may be positioned between the first base of the poly-A signal and 250 nucleotides upstream of the first base. The region may be positioned between the first base of the poly-A signal and 150 nucleotides upstream of the first base. The region may be positioned between the first base of the poly-A signal and 100 nucleotides upstream of the first base. The region may be positioned between the first base of the poly-A signal and 50 nucleotides upstream of the first base. The region may be positioned between the first base of the poly-A signal and 20 nucleotides upstream of the first base. In some embodiments, the region is positioned between the last nucleotide base of a promoter sequence and the first nucleotide base of a poly-A signal sequence.

In some cases, the region may be positioned downstream of the last base of the poly-A signal of a transgene. The region may be between the last base of the poly-A signal and a position 2000 nucleotides downstream of the last base. The region may be between the last base of the poly-A signal and a position 1000 nucleotides downstream of the last base. The region may be between the last base of the poly-A signal and a position 500 nucleotides downstream of the last base. The region may be between the last base of the poly-A signal and a position 250 nucleotides downstream of the last base. The region may be between the last base of the poly-A signal and a position 150 nucleotides downstream of the last base.

It should be appreciated that in cases where a transgene encodes more than one polypeptide, each polypeptide may be positioned in any suitable location within the transgene. For example, a nucleic acid encoding a first polypeptide may be positioned in an intron of the transgene and a nucleic acid sequence encoding a second polypeptide may be positioned in another untranslated region (e.g., between the last codon of a protein coding sequence and the first base of the poly-A signal of the transgene).

A “promoter” refers to a DNA sequence recognized by the synthetic machinery of the cell, or introduced synthetic machinery, required to initiate the specific transcription of a gene. The phrases “operatively positioned,” “under control” or “under transcriptional control” means that the promoter is in the correct location and orientation in relation to the nucleic acid to control RNA polymerase initiation and expression of the gene.

For nucleic acids encoding proteins, a polyadenylation sequence generally is inserted following the transgene sequences and before the 3′ AAV ITR sequence. A rAAV construct useful in the present disclosure may also contain an intron, desirably located between the promoter/enhancer sequence and the transgene. One possible intron sequence is derived from SV-40, and is referred to as the SV-40 T intron sequence. Another vector element that may be used is an internal ribosome entry site (IRES). An IRES sequence is used to produce more than one polypeptide from a single gene transcript. An IRES sequence would be used to produce a protein that contain more than one polypeptide chains. Selection of these and other common vector elements are conventional and many such sequences are available [see, e.g., Sambrook et al., and references cited therein at, for example, pages 3.18 3.26 and 16.17 16.27 and Ausubel et al., Current Protocols in Molecular Biology, John Wiley & Sons, New York, 1989]. In some embodiments, a Foot and Mouth Disease Virus 2A sequence is included in polyprotein; this is a small peptide (approximately 18 amino acids in length) that has been shown to mediate the cleavage of polyproteins (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459). The cleavage activity of the 2A sequence has previously been demonstrated in artificial systems including plasmids and gene therapy vectors (AAV and retroviruses) (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459; de Felipe, P et al., Gene Therapy, 1999; 6: 198-208; de Felipe, P et al., Human Gene Therapy, 2000; 11: 1921-1931.; and Klump, H et al., Gene Therapy, 2001; 8: 811-817).

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al., Cell, 41: 521-530 (1985)], the SV40 promoter, the dihydrofolate reductase promoter, the 3-actin promoter, the phosphoglycerol kinase (PGK) promoter, and the EF1α promoter [Invitrogen]. In some embodiments, a promoter is a P2 promoter. In some embodiments, a promoter is a chicken (3-actin (CBA) promoter. In some embodiments, a promoter is two CBA promoters. In some embodiments, a promoter is two CBA promoters separated by a CMV enhancer.

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al., Proc. Natl. Acad. Sci. USA, 89: 5547-5551 (1992)), the tetracycline-inducible system (Gossen et al., Science, 268: 1766-1769 (1995), see also Harvey et al., Curr. Opin. Chem. Biol., 2: 512-518 (1998)), the RU486-inducible system (Wang et al., Nat. Biotech., 15: 239-243 (1997) and Wang et al., Gene Ther., 4: 432-441 (1997)) and the rapamycin-inducible system (Magari et al., J. Clin. Invest., 100: 2865-2872 (1997)). Still other types of inducible promoters which may be useful in this context are those which are regulated by a specific physiological state, e.g., temperature, acute phase, a particular differentiation state of the cell, or in replicating cells only.

In another embodiment, the native promoter for the transgene will be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. Exemplary tissue-specific regulatory sequences include, but are not limited to the following tissue specific promoters: a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (α-MHC) promoter, or a cardiac Troponin T (cTnT) promoter. Other exemplary promoters include Beta-actin promoter, hepatitis B virus core promoter, Sandig et al., Gene Ther., 3: 1002-9 (1996); alpha-fetoprotein (AFP) promoter, Arbuthnot et al., Hum. Gene Ther., 7: 1503-14 (1996)), bone osteocalcin promoter (Stein et al., Mol. Biol. Rep., 24: 185-96 (1997)); bone sialoprotein promoter (Chen et al., J. Bone Miner. Res., 11: 654-64 (1996)), CD2 promoter (Hansal et al., J. Immunol., 161: 1063-8 (1998); immunoglobulin heavy chain promoter; T cell receptor α-chain promoter, neuronal such as neuron-specific enolase (NSE) promoter (Andersen et al., Cell. Mol. Neurobiol., 13: 503-15 (1993)), neurofilament light-chain gene promoter (Piccioli et al., Proc. Natl. Acad. Sci. USA, 88: 5611-5 (1991)), and the neuron-specific vgf gene promoter (Piccioli et al., Neuron, 15: 373-84 (1995)), among others which will be apparent to the skilled artisan.

Aspects of the disclosure relate to an isolated nucleic acid comprising more than one promoter (e.g., 2, 3, 4, 5, or more promoters). For example, in the context of a construct having a transgene comprising a first region encoding a protein and an second region encoding a protein it may be desirable to drive expression of the first protein coding region using a first promoter sequence (e.g., a first promoter sequence operably linked to the protein coding region), and to drive expression of the second protein coding region with a second promoter sequence (e.g., a second promoter sequence operably linked to the second protein coding region). Generally, the first promoter sequence and the second promoter sequence can be the same promoter sequence or different promoter sequences. In some embodiments, the first promoter sequence (e.g., the promoter driving expression of the protein coding region) is a RNA polymerase III (pol III) promoter sequence. Non-limiting examples of pol III promoter sequences include U6 and H1 promoter sequences. In some embodiments, the second promoter sequence (e.g., the promoter sequence driving expression of the second protein) is a RNA polymerase II (pol II) promoter sequence. Non-limiting examples of pol II promoter sequences include T7, T3, SP6, RSV, and cytomegalovirus promoter sequences. In some embodiments, a pol III promoter sequence drives expression of the first protein coding region. In some embodiments, a pol II promoter sequence drives expression of the second protein coding region.

In some embodiments, the nucleic acid comprises a transgene that encodes a protein. The protein can be a therapeutic protein (e.g., a peptide, protein, or polypeptide useful for the treatment or prevention of disease states in a mammalian subject) or a reporter protein. In some embodiments, the therapeutic protein is useful for treatment or prevention of lysosomal storage diseases such as Tay-Sachs or Sandhoff disease, including, but not limited to, Hexosaminidase A (HexA) and Hexosaminidase B (HexB).

### Bicistronic Nucleic Acid Constructs

Some aspects of this invention provide bicistronic nucleic acid constructs. The term “cistron” refers to a nucleic acid cassette sufficient for expression of a gene product. In some embodiments, a cistron is an expression cassette. Accordingly, some aspects of this invention provide nucleic acid constructs comprising two or more cistrons, for example, two or more expression cassettes. The term “expression cassette” refers to a nucleic construct comprising nucleic acid elements sufficient for the expression of a gene product. Typically, an expression cassette comprises a nucleic acid encoding a gene product operatively linked to a promoter sequence. Encoding sequences can be operatively linked to regulatory sequences in sense or antisense orientation. In some embodiments, the promoter is a heterologous promoter. The term “heterologous promoter”, as used herein, refers to a promoter that does is not found to be operatively linked to a given encoding sequence in nature. In some embodiments, an expression cassette may comprise additional elements, for example, an intron, an enhancer, a polyadenylation site, a woodchuck hepatitis virus post-transcriptional regulatory element (WPRE), and/or other elements known to affect expression levels of the encoding sequence. Without wishing to be bound by theory, inclusion of an intron in an expression cassette, for example, between the transcriptional start site and an encoding nucleic acid sequence, for example, a protein-encoding cDNA sequence, is believed to result in increased expression levels of the encoding nucleic acid and the encoded gene product as compared to an expression construct not including an intron.

The term “intron” refers to a nucleic acid sequence in an expression cassette that is removed after transcription of a primary transcript by a cellular process termed splicing. Intron sequences generally comprise a splice donor and a splice acceptor and sequences of such donor and acceptor sites are well known to those of skill in the art. “Chimeric intron” as used herein, are composed of nucleic acid sequences from two or more different sources.

Some aspects of this invention provide bicistronic expression constructs comprising two or more expression cassettes in various configurations.

In different embodiments, bicistronic expression constructs are provided in which the expression cassettes are positioned in different ways. For example, in some embodiments, a multicistronic expression construct is provided in which a first expression cassette is positioned adjacent to a second expression cassette. In some embodiments, the first expression cassette and the second expression cassette are operably linked by a bidirectional promoter, wherein the first expression cassette and the second expression cassette are flanked by adeno-associated virus (AAV) inverted terminal repeats (ITRs).

In different embodiments, bicistronic expression constructs are provided in which the expression cassettes are oriented in different ways. For example, in some embodiments, bicistronic expression construct is provided comprising a first and a second expression cassette in opposite orientations.

The term “orientation” as used herein in connection with expression cassettes, refers to the directional characteristic of a given cassette or structure. In some embodiments, an expression cassette harbors a promoter 5′ of the encoding nucleic acid sequence, and transcription of the encoding nucleic acid sequence runs from the 5′ terminus to the 3′ terminus of the sense strand, making it a directional cassette (e.g., 5′-promoter/(intron)/encoding sequence-3′). Since virtually all expression cassettes are directional in this sense, those of skill in the art can easily determine the orientation of a given expression cassette in relation to a second nucleic acid structure, for example, a second expression cassette, a viral genome, or, if the cassette is comprised in an AAV construct, in relation to an AAV ITR.

For example, if a given nucleic acid construct comprises two expression cassettes in the configuration 5′-promoter 1/encoding sequence 1---promoter2/encoding sequence 2-3′,
>>>>>>>>>>>>>>>>>>>>>>> >>>>>>>>>>>>>>>>>>>>>>>
the expression cassettes are in the same orientation, the arrows indicate the direction of transcription of each of the cassettes. For another example, if a given nucleic acid construct comprises a sense strand comprising two expression cassettes in the configuration
5′-promoter 1/encoding sequence 1---encoding sequence 2/promoter 2-3′,
>>>>>>>>>>>>>>>>>>>>>>> <<<<<<<<<<<<<<<<<<<<<<<
the expression cassettes are in opposite orientation to each other and, as indicated by the arrows, the direction of transcription of the expression cassettes, are opposed. In this example, the strand shown comprises the antisense strand of promoter 2 and encoding sequence 2.

For another example, if an expression cassette is comprised in an AAV construct, the cassette can either be in the same orientation as an AAV ITR or a second expression cassette (e.g., transcription of the expression cassette proceeds in the same direction as transcription of the AAV ITR or second expression cassette), or in opposite orientation (e.g., transcription of the expression cassette proceeds in the opposite direction (e.g., distally) as transcription of the AAV ITR or second expression cassette). AAV ITRs are directional. For example, an AAV construct comprising a 5′ITR exemplified in FIG. 1 (top) would be in the same orientation as the HexA expression cassette expression cassette. In another example, an AAV construct comprising the 5′ITR exemplified in FIG. 1 (bottom), would be in the opposite orientation as the HexA expression cassette expression cassette.

A large body of evidence suggests that bicistronic expression constructs often do not achieve optimal expression levels as compared to expression systems containing only one cistron. One of the suggested causes of sub-par expression levels achieved with bicistronic expression constructs comprising two or more promoter elements is the phenomenon of promoter interference (see, e.g., Curtin J A, Dane A P, Swanson A, Alexander I E, Ginn S L. Bidirectional promoter interference between two widely used internal heterologous promoters in a late-generation lentiviral construct. Gene Ther. 2008 March; 15(5): 384-90; and Martin-Duque P, Jezzard S, Kaftansis L, Vassaux G. Direct comparison of the insulating properties of two genetic elements in an adenoviral vector containing two different expression cassettes. Hum Gene Ther. 2004 October; 15(10): 995-1002; both references incorporated herein by reference for disclosure of promoter interference phenomenon). Various strategies have been suggested to overcome the problem of promoter interference, for example, by producing bicistronic expression constructs comprising only one promoter driving transcription of multiple encoding nucleic acid sequences separated by internal ribosomal entry sites, or by separating cistrons comprising their own promoter with transcriptional insulator elements. All suggested strategies to overcome promoter interference are burdened with their own set of problems, though. For example, single-promoter driven expression of multiple cistrons usually results in uneven expression levels of the cistrons. Further some promoters cannot efficiently be isolated and isolation elements are not compatible with some gene transfer vectors, for example, some retroviral vectors.

In some embodiments, a bicistronic expression construct is provided that allows efficient expression of a first encoding nucleic acid sequence driven by a first promoter and of a second encoding nucleic acid sequence driven by a second promoter without the use of transcriptional insulator elements. Various configurations of such bicistronic expression constructs are provided herein, for example, expression constructs harboring a first expression cassette comprising an intron and a second expression cassette comprising an intron, wherein the first expression cassette and second expression cassette are under the control of separate promoters located proximal to the AAV ITRs that flank the first expression cassette and the second expression cassette. In some embodiments, the first expression cassette and the second expression cassette are operably linked by a bidirectional promoter and are flanked by AAV ITRs.

In some embodiments, bicistronic expression constructs are provided allowing for efficient expression of two or more encoding nucleic acid sequences. In some embodiments, the bicistronic expression construct comprises two expression cassettes. In some embodiments, a first expression cassette of a bicistronic expression construct as provided herein comprises an RNA polymerase II promoter and a second expression cassette comprises an RNA polymerase III promoter. In some embodiments, a first expression cassette comprises a P2 promoter and a second expression cassette comprises a P2 promoter. In some embodiments, a first expression cassette and a second expression cassette are operably linked by a bidirectional promoter. In some embodiments, the bicistronic expression construct provided is a recombinant AAV (rAAV) construct.

Recombinant Adeno-Associated Viruses (rAAVs)

In some aspects, the disclosure provides isolated adeno-associated viruses (AAVs). As used herein with respect to AAVs, the term “isolated” refers to an AAV that has been artificially produced or obtained. Isolated AAVs may be produced using recombinant methods. Such AAVs are referred to herein as “recombinant AAVs”. Recombinant AAVs (rAAVs) preferably have tissue-specific targeting capabilities, such that a nuclease and/or transgene of the rAAV will be delivered specifically to one or more predetermined tissue(s). The AAV capsid is an important element in determining these tissue-specific targeting capabilities. Thus, an rAAV having a capsid appropriate for the tissue being targeted can be selected.

Methods for obtaining recombinant AAVs having a desired capsid protein are well known in the art. (See, for example, US 2003/0138772), the contents of which are incorporated herein by reference in their entirety). Typically the methods involve culturing a host cell which contains a nucleic acid sequence encoding an AAV capsid protein; a functional rep gene; a recombinant AAV vector composed of AAV inverted terminal repeats (ITRs) and a transgene; and sufficient helper functions to permit packaging of the recombinant AAV vector into the AAV capsid proteins. In some embodiments, capsid proteins are structural proteins encoded by the cap gene of an AAV. AAVs comprise three capsid proteins, virion proteins 1 to 3 (named VP1, VP2 and VP3), all of which are transcribed from a single cap gene via alternative splicing. In some embodiments, the molecular weights of VP1, VP2 and VP3 are respectively about 87 kDa, about 72 kDa and about 62 kDa. In some embodiments, upon translation, capsid proteins form a spherical 60-mer protein shell around the viral genome. In some embodiments, the functions of the capsid proteins are to protect the viral genome, deliver the genome and interact with the host. In some aspects, capsid proteins deliver the viral genome to a host in a tissue specific manner.

In some embodiments, an AAV capsid protein is of an AAV serotype selected from the group consisting of AAV1, AAV2, AAV3, AAV4, AAV5, AAV6, AAV7, AAV8, AAVrh8, AAV9, AAV10, AAVrh10, and AAV.PHP.B. In some embodiments, an AAV capsid protein is of a serotype derived from a non-human primate, for example AAVrh8 serotype. In some embodiments, an AAV capsid protein is of a serotype derived for broad and efficient CNS transduction, for example AAV.PHP.B. In some embodiments, the capsid protein is of AAV serotype 9.

The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters. Still other stable host cells may be generated by one of skill in the art.

In some embodiments, the instant disclosure relates to a host cell containing a nucleic acid that comprises a coding sequence encoding a protein or proteins (e.g., HEXA and HEXB proteins). In some embodiments, the host cell is a mammalian cell, a yeast cell, a bacterial cell, an insect cell, a plant cell, or a fungal cell.

The recombinant AAV vector, rep sequences, cap sequences, and helper functions required for producing the rAAV of the disclosure may be delivered to the packaging host cell using any appropriate genetic element (vector). The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al., Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. See, e.g., K. Fisher et al., J. Virol., 70: 520-532 (1993) and U.S. Pat. No. 5,478,745.

In some embodiments, recombinant AAVs may be produced using the triple transfection method (described in detail in U.S. Pat. No. 6,001,650). Typically, the recombinant AAVs are produced by transfecting a host cell with an AAV vector (comprising a transgene flanked by ITR elements) to be packaged into AAV particles, an AAV helper function vector, and an accessory function vector. An AAV helper function vector encodes the “AAV helper function” sequences (e.g., rep and cap), which function in trans for productive AAV replication and encapsidation. Preferably, the AAV helper function vector supports efficient AAV vector production without generating any detectable wild-type AAV virions (e.g., AAV virions containing functional rep and cap genes). Non-limiting examples of vectors suitable for use with the present disclosure include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (e.g., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

In some aspects, the disclosure provides transfected host cells. The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells.

A “host cell” refers to any cell that harbors, or is capable of harboring, a substance of interest. Often a host cell is a mammalian cell. A host cell may be used as a recipient of an AAV helper construct, an AAV minigene plasmid, an accessory function vector, or other transfer DNA associated with the production of recombinant AAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

As used herein, the term “vector” includes any genetic element, such as a plasmid, phage, transposon, cosmid, chromosome, artificial chromosome, virus, virion, etc., which is capable of replication when associated with the proper control elements and which can transfer gene sequences between cells. Thus, the term includes cloning and expression vehicles, as well as viral vectors. In some embodiments, useful vectors are contemplated to be those vectors in which the nucleic acid segment to be transcribed is positioned under the transcriptional control of a promoter. A “promoter” refers to a DNA sequence recognized by the synthetic machinery of the cell, or introduced synthetic machinery, required to initiate the specific transcription of a gene. The phrases “operatively positioned,” “under control” or “under transcriptional control” means that the promoter is in the correct location and orientation in relation to the nucleic acid to control RNA polymerase initiation and expression of the gene. The term “expression vector or construct” means any type of genetic construct containing a nucleic acid in which part or all of the nucleic acid encoding sequence is capable of being transcribed. In some embodiments, expression includes transcription of the nucleic acid, for example, to generate a biologically-active polypeptide product from a transcribed gene. The foregoing methods for packaging recombinant vectors in desired AAV capsids to produce the rAAVs of the disclosure are not meant to be limiting and other suitable methods will be apparent to the skilled artisan.

### Methods for Treating Lysosomal Storage Diseases

Methods for delivering a transgene to a subject are provided by the disclosure. A subject may be any mammal, for example a human, non-human primate, rodent, dog, cat, horse, pig, etc. In some embodiments, a subject is a human. The methods typically involve administering to a subject an effective amount of isolated nucleic acid encoding hexosaminidase A (HEXA) and hexosaminidase B (HEXB) (e.g., wild-type HEXA and/or HEXB, codon optimized HEXA and/or HEXB, or any combination of the foregoing) proteins capable of compensating for mutant HEXA and HEXB proteins in a subject. HEXA and HEXB proteins form the heterodimeric isozyme beta-hexosaminidase A, which is the lysosomal protein/enzyme in human that breaks down GM2 ganglioside. As such, bi-allelic mutations in a HEXA gene and/or a HEXB gene generally result in the toxic accumulation of GM2 ganglioside and lysosomal storage disorders.

As used herein, a “lysosomal storage disorder” refers to an inherited metabolic disease characterized by an abnormal build-up of GM2 ganglioside in cells, particularly neurons, resulting from bi-allelic mutations in a HEXA gene and/or a HEXB gene. In some embodiments, a subject is characterized by having a mutation in a HEXA gene resulting in reduced or loss of function of the beta-hexosaminidase A protein of the subject. In some embodiments, a subject is characterized by having a mutation in a HEXB gene resulting in a reduced or loss of function of the beta-hexosaminidase A protein of the subject. “Bi-allelic mutations” refers to both copies of a gene, in this case either HEXA and/or HEXB, possessing alterations in amino acid sequence. The progressive build-up of GM2 ganglioside in lysosomes leads to the destruction of neurons. In some embodiments, the lysosomal storage disorder is Tay-Sachs disease (TSD), a condition caused by reduced or loss of function of the HEXA protein. In some embodiments, the lysosomal storage disorder is Sandhoff disease (SD), a condition caused by reduced or loss of function of the HEXB protein.

An “effective amount” of a substance is an amount sufficient to produce a desired effect. In some embodiments, an effective amount of an isolated nucleic acid is an amount sufficient to transfect (or infect in the context of rAAV mediated delivery) a sufficient number of target cells of a target tissue of a subject. In some embodiments, a target tissue is central nervous system (CNS) tissue (e.g., brain tissue, spinal cord tissue, cerebrospinal fluid (CSF), etc.). In some embodiments, an effective amount of an isolated nucleic acid (e.g., which may be delivered via an rAAV) may be an amount sufficient to have a therapeutic benefit in a subject, e.g., to compensate for reduction or loss of function of a protein resulting from mutation of a gene (e.g., HEXA or HEXB), to extend the lifespan of a subject, to improve in the subject one or more symptoms of disease (e.g., a symptom of TSD or SD), etc. The effective amount will depend on a variety of factors such as, for example, the species, age, weight, health of the subject, and the tissue to be targeted, and may thus vary among subject and tissue as described elsewhere in the disclosure.

In some embodiments an isolated nucleic acid or rAAV as described herein is useful to treat a lysosomal storage disease, for example Tay-Sachs disease or Sandhoff disease. As used herein, the term “treating” refers to the application or administration of a composition encoding an isolated nucleic acid (e.g., an rAAV comprising an isolated nucleic acid) as described herein to a subject, who has a lysosomal storage disease, a symptom of a lysosomal storage disease, or a predisposition toward a lysosomal storage disease (e.g., one or more mutations in the HEXA gene, HEXB gene, etc.), with the purpose to cure, heal, alleviate, relieve, alter, remedy, ameliorate, improve, or affect the disorder, the symptom of the disease, or the predisposition toward the lysosomal storage disease.

Alleviating a lysosomal storage disease includes delaying the development or progression of the disease, or reducing disease severity. Alleviating the disease does not necessarily require curative results. As used therein, “delaying” the development of a lysosomal storage disease means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a lysosomal storage disease includes initial onset and/or recurrence.

### Modes of Administration

The rAAVs of the disclosure may be delivered to a subject in compositions according to any appropriate methods known in the art. For example, an rAAV, preferably suspended in a physiologically compatible carrier (e.g., in a composition), may be administered to a subject, i.e. host animal, such as a human, mouse, rat, cat, dog, sheep, rabbit, horse, cow, goat, pig, guinea pig, hamster, chicken, turkey, or a non-human primate (e.g., Macaque). In some embodiments a host animal does not include a human.

Delivery of the rAAVs to a mammalian subject may be by, for example, intramuscular injection or by administration into the bloodstream of the mammalian subject. Administration into the bloodstream may be by injection into a vein, an artery, or any other vascular conduit. In some embodiments, the rAAVs are administered into the bloodstream by way of isolated limb perfusion, a technique well known in the surgical arts, the method essentially enabling the artisan to isolate a limb from the systemic circulation prior to administration of the rAAV virions. A variant of the isolated limb perfusion technique, described in U.S. Pat. No. 6,177,403, can also be employed by the skilled artisan to administer the virions into the vasculature of an isolated limb to potentially enhance transduction into muscle cells or tissue. Moreover, in certain instances, it may be desirable to deliver the virions to the CNS of a subject. By “CNS” is meant all cells and tissue of the brain and spinal cord of a vertebrate. Thus, the term includes, but is not limited to, neuronal cells, glial cells, astrocytes, cerebrospinal fluid (CSF), interstitial spaces, bone, cartilage and the like. Recombinant AAVs may be delivered directly to the CNS or brain by injection into, e.g., the ventricular region, as well as to the striatum (e.g., the caudate nucleus or putamen of the striatum), thalamus, spinal cord and neuromuscular junction, or cerebellar lobule, with a needle, catheter or related device, using neurosurgical techniques known in the art, such as by stereotactic injection (see, e.g., Stein et al., J Virol 73: 3424-3429, 1999; Davidson et al., PNAS 97: 3428-3432, 2000; Davidson et al., Nat. Genet. 3: 219-223, 1993; and Alisky and Davidson, Hum. Gene Ther. 11: 2315-2329, 2000). In some embodiments, an rAAV as described in the disclosure are administered by intravenous injection. In some embodiments, rAAVs are administered by intracerebral injection. In some embodiments, rAAVs are administered by intrathecal injection. In some embodiments, rAAVs are administered by intrastriatal injection. In some embodiments, rAAVs are delivered by intracranial injection. In some embodiments, rAAVs are delivered by cisterna magna injection. In some embodiments, the rAAV are delivered by cerebral lateral ventricle injection.

Aspects of the instant disclosure relate to compositions comprising a recombinant AAV comprising a capsid protein and a nucleic acid encoding a transgene, wherein the transgene comprises a nucleic acid sequence encoding one or more proteins. In some embodiments, each protein comprises a sequence set forth in any one of SEQ ID NO: 1 or 2. In some embodiments, the nucleic acid further comprises AAV ITRs. In some embodiments, the rAAV comprises an rAAV vector represented by the sequence set forth in any one of SEQ ID NOs: 3-9, or a portion thereof. In some embodiments, a composition further comprises a pharmaceutically acceptable carrier.

The compositions of the disclosure may comprise an rAAV alone, or in combination with one or more other viruses (e.g., a second rAAV encoding having one or more different transgenes). In some embodiments, a composition comprises 1, 2, 3, 4, 5, 6, 7, 8, 9, 10, or more different rAAVs each having one or more different transgenes.

Suitable carriers may be readily selected by one of skill in the art in view of the indication for which the rAAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure.

Optionally, the compositions of the disclosure may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, parachlorophenol, and poloxamers (non-ionic surfactants) such as Pluronic® F-68. Suitable chemical stabilizers include gelatin and albumin.

The rAAVs are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected organ (e.g., intraportal delivery to the liver), oral, inhalation (including intranasal and intratracheal delivery), intraocular, intravenous, intramuscular, subcutaneous, intradermal, intratumoral, and other parental routes of administration. Routes of administration may be combined, if desired.

The dose of rAAV virions required to achieve a particular “therapeutic effect,” e.g., the units of dose in genome copies/per kilogram of body weight (GC/kg), will vary based on several factors including, but not limited to: the route of rAAV virion administration, the level of gene or RNA expression required to achieve a therapeutic effect, the specific disease or disorder being treated, and the stability of the gene or RNA product. One of skill in the art can readily determine a rAAV virion dose range to treat a patient having a particular disease or disorder based on the aforementioned factors, as well as other factors that are well known in the art.

An effective amount of an rAAV is an amount sufficient to target infect an animal, target a desired tissue. In some embodiments, an effective amount of an rAAV is administered to the subject during a pre-symptomatic stage of the lysosomal storage disease. In some embodiments, the pre-symptomatic stage of the lysosomal storage disease occurs between birth (e.g., perinatal) and 4-weeks of age.

In some embodiments, rAAV compositions are formulated to reduce aggregation of AAV particles in the composition, particularly where high rAAV concentrations are present (e.g., ˜10¹³ GC/mL or more). Methods for reducing aggregation of rAAVs are well known in the art and, include, for example, addition of surfactants, pH adjustment, salt concentration adjustment, etc. (See, e.g., Wright F R, et al., Molecular Therapy (2005) 12, 171-178, the contents of which are incorporated herein by reference.)

Formulation of pharmaceutically-acceptable excipients and carrier solutions is well-known to those of skill in the art, as is the development of suitable dosing and treatment regimens for using the particular compositions described herein in a variety of treatment regimens.

Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver the rAAV-based therapeutic constructs in suitably formulated pharmaceutical compositions disclosed herein either subcutaneously, intraopancreatically, intranasally, parenterally, intravenously, intramuscularly, intrathecally, or orally, intraperitoneally, or by inhalation. In some embodiments, the administration modalities as described in U.S. Pat. Nos. 5,543,158; 5,641,515 and 5,399,363 (each specifically incorporated herein by reference in its entirety) may be used to deliver rAAVs. In some embodiments, a preferred mode of administration is by portal vein injection.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the active rAAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The rAAV compositions disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present disclosure into suitable host cells. In particular, the rAAV vector delivered transgenes may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the rAAV constructs disclosed herein. The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 A, containing an aqueous solution in the core.

Alternatively, nanocapsule formulations of the rAAV may be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

In addition to the methods of delivery described above, the following techniques are also contemplated as alternative methods of delivering the rAAV compositions to a host. Sonophoresis (i.e., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

### EXAMPLES

AAV vectors encoding both alpha and beta-subunits of hexosaminidase (HexA and HexB, respectively) have been developed (FIG. 1) using two design principles for bicistronic vectors. In AAV-BiP2i-HexAB (P2I), the transgene cassettes are positioned in opposite orientations with the promoters driving expression from the ends of the genome. The other vector, AAV-BiCBA-HexAB (Bic), uses a bidirectional promoter composed of a single CMV enhancer flanked by the minimal chicken β-actin (CBA) promoter to drive expression in opposite directions from the center of the genome.

Short-term studies using AAV-PHP.B vectors demonstrated efficacy. Although AAV9 is considerably less efficient than AAV-PHP.B for systemic CNS gene delivery in mice, it is also a capsid for which there is considerable clinical experience with an excellent safety track record. Moreover, systemic delivery of AAV9 has been observed to mediate transformative therapeutic results in spinal muscular atrophy (SMA) patients. For these reasons, it was included in therapeutic experiments for a total of four vectors being tested.

Four week-old Sandhoff mice (Hex^{-/-}) were treated systemically (tail vein injection) with 1×10¹² vg (n=8) or 4×10¹² vg (n=14) of AAV9 vectors (Bic or P2I) or 3×10¹¹ vg (n=8) or 1×10¹² vg (n=14) of AAV-PHP.B vectors (Bic or P2I) encoding mouse HexA and HexB proteins. Age matched PBS-injected SD (n=6) and wild-type (n=14) mice were used as controls. All cohorts were composed of equal numbers of males and females. A subset of mice (n=6) in the high dose cohorts and normal controls were sacrificed at 150 days of age, or humane endpoint to assess efficacy using biochemical (enzyme activity and GM2 ganglioside content) and histological outcome measures, while the remaining mice were used for survival analysis. PBS-treated SD mice were euthanized when they reached the humane endpoint (all before 150 days of age). Humane endpoint was determined by any of the following parameters: inability to right themselves for 30 seconds when placed in supine position, paralysis of one hindlimb, or more than 15% body weight loss from peak weight.

### Example 1: Behavioral Performance of AAV-Treated SD Mice Remained Stable Over Time

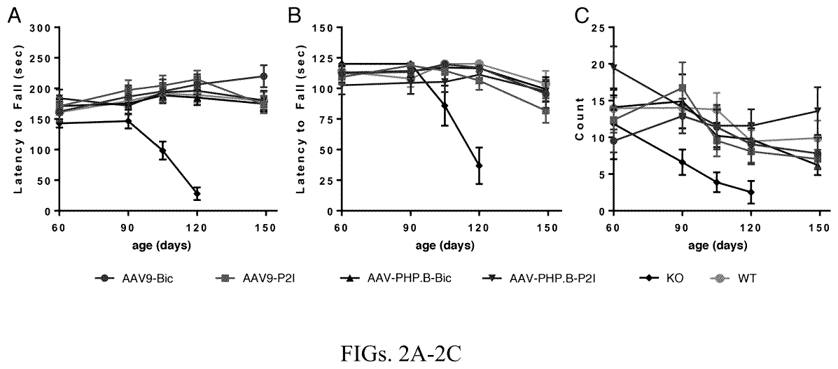

The behavioral performance of AAV-treated SD and control (KO) mice was assessed using the rotarod and inverted screen tests at 60, 90, 105, 120, and 149 days of age. The motor coordination and performance of AAV-treated SD mice remained comparable to normal controls in the accelerated rotarod (FIG. 2A) and inverted screen tests (FIGS. 2B-2C) tests up to the last time point at 149 days of age. The performance of untreated SD mice declined over time and no animals remained alive at the last time point.

### Example 2: Systemic AAV Treatment Reduces GM2 Ganglioside Content Throughout the Central Nervous System of SD Mice

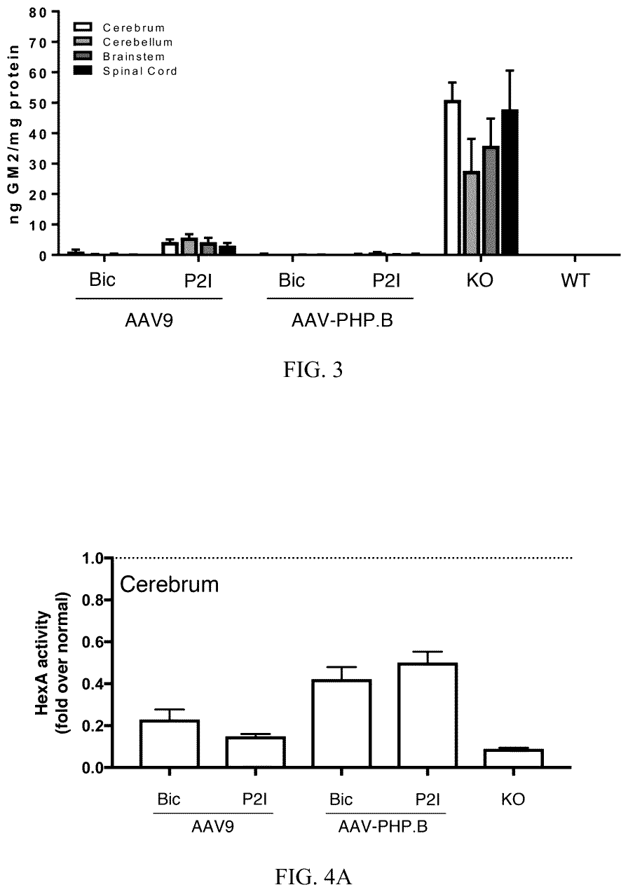

The GM2 ganglioside content in the brain, cerebellum, brainstem, and spinal cord of AAV-treated SD mice (high dose) at 150 days of age was significantly lower than in untreated SD mice at the humane endpoint, and the barely detectable in AAV9-Bic and both AAV-PHP.B treatment groups (FIG. 3). The GM2 levels remained above background in AAV9-P2I treated animals.

### Example 3: Increased Hexosaminidase Activity in Brain and Liver of AAV-Treated SD Mice

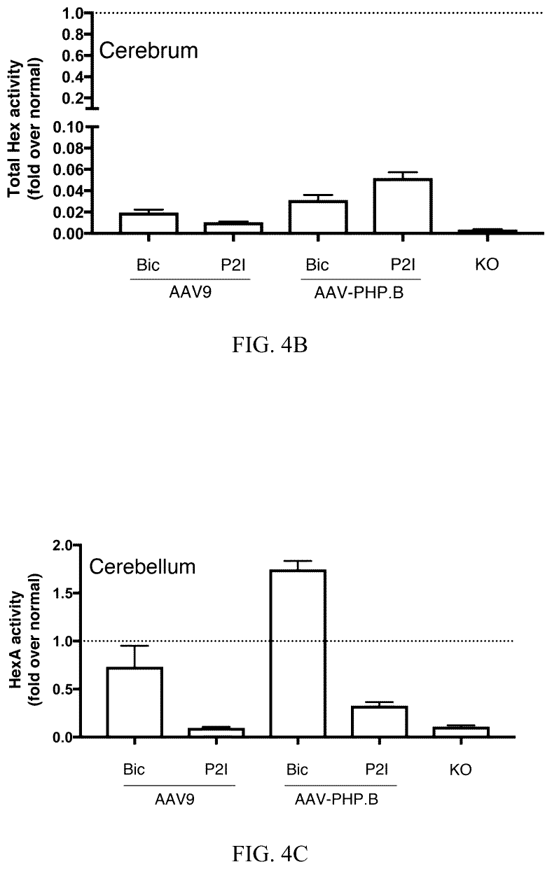

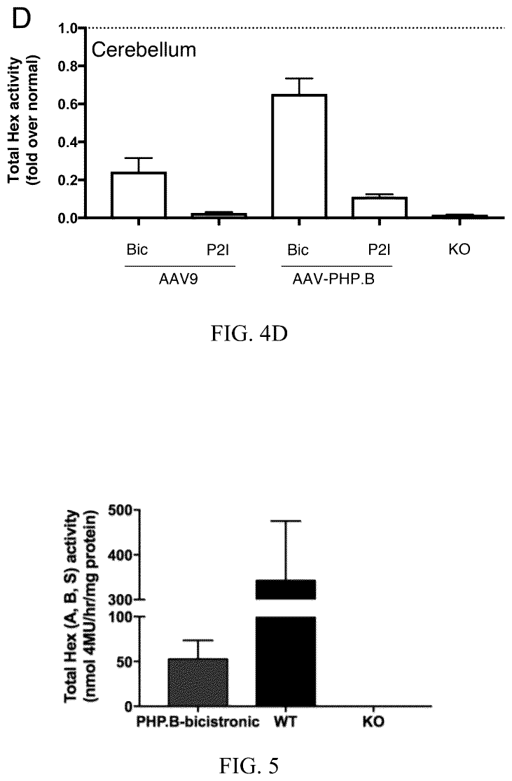

Hexosaminidase activity in the cerebrum and cerebellum (FIGS. 4A-4D) were consistent with the findings for GM2 ganglioside content. The enzyme activity in the brain of AAV9-P2I treated mice was consistently lower than in other groups, but nonetheless restoration of ˜10% of normal HexA activity appears sufficient to significantly reduce GM2 ganglioside content in the cerebrum (FIG. 3). Restoring HexA activity in the cerebrum to ˜20% as in AAV9-Bic treated animals (FIG. 4A) appears sufficient to largely eliminate GM2 ganglioside storage (FIG. 3). Total hexosaminidase (HexA, HexB, HexS) activity in the liver was also assessed following systemic administration of AAV-PHP.Bic using the artificial substrate MUG (FIG. 5). In the liver, the total hexosaminidase activity was restored to 15% of normal.

### Example 4: Systemic AAV Treatment Extends Survival of SD Mice

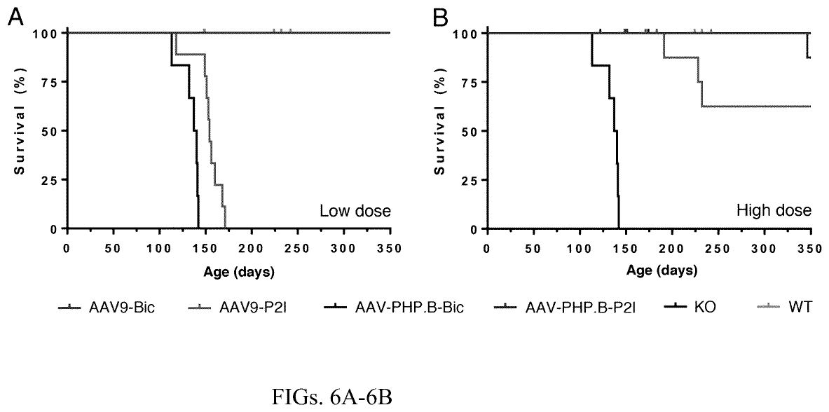

All SD mice injected with AAV-Bic vectors remain alive past 350 days in the low (3×10¹¹ vg) and high dose cohorts (1×10¹² vg) (FIG. 6A-6B). Systemic AAV9-P2I treatment had a marginal impact of survival at the low dose (1×10¹² vg), but it increased at the high dose (4×10¹² vg) (FIG. 6A-6B). The impact of AAV9-P2I treatment on survival is consistent with the CNS biochemical findings where GM2 ganglioside levels were reduced in relation to untreated control SD mice (FIG. 3), but remained detectable, unlike in other AAV treatment groups or normal cohorts.

### Example 5: Systemic AAV Treatment Restores Hex Activity Throughout Brain of SD Mice

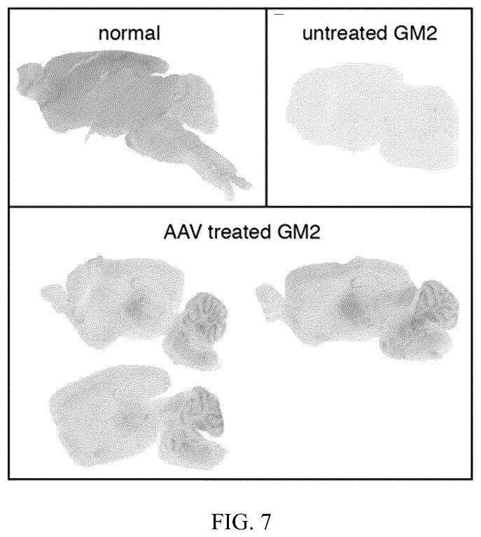

Analysis of Hexosaminidase distribution in the brain following delivery of AAV-PHP.B-BiCBA-HexAB using a Hexosaminidase specific histochemical stain showed restoration of enzyme activity throughout the brain (FIG. 7) indicating that this approach, in some embodiments, mediates a therapeutic impact.

### Example 6: CSF Delivery of AAV Gene Therapy for Tay-Sachs and Sandhoff Disease

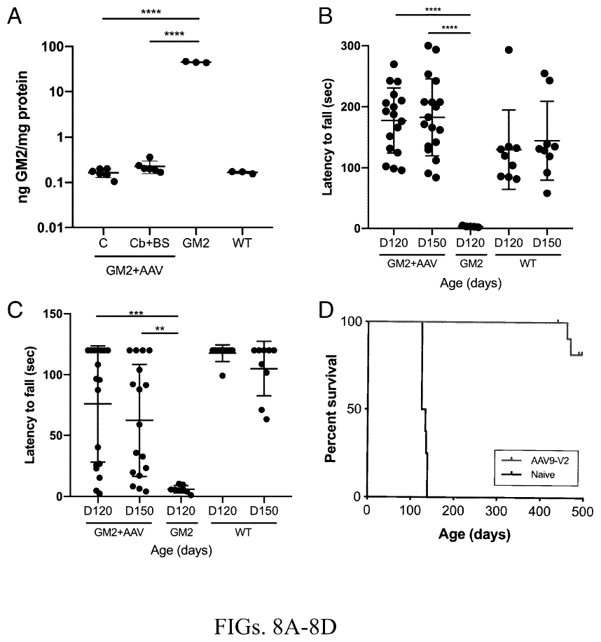

Neonatal GM2 mice (n=31 mice) received bilateral injections of AAV9-BiCB-HexAB (7.25×10¹⁰ vg) into the brain lateral ventricles (2 μl per side). Control GM2 mice (n=8) received bilateral injection of phosphate buffered saline. At one month of age the GM2 ganglioside content in the brain of AAV-treated GM2 mice (n=6) was identical to that in normal animals, which is less than 0.5% of the level found in age matched untreated GM2 mice (FIG. 8A). The motor performance of AAV treated GM2 mice in two tasks, rotarod (FIG. 8B) and inverted screen (FIG. 8C), was significantly improved compared to untreated GM2 mice at 120 days of age and remained stable to 150 days of age. Presently the majority of AAV treated GM2 mice remain alive past 400 days of age compared to a median survival of 129.5 days for untreated GM2 mice (FIG. 8D).

### Example 7: AAV9-BiCB-HexAB Vector Injected into CSF Through Intrathecal Delivery in Young Adult GM2 Mice

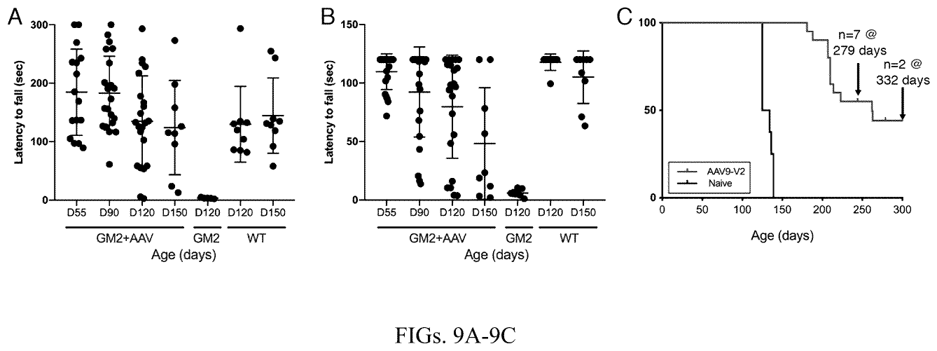

Lumbar intrathecal (LIT) injection of 1×10¹² vg AAV9-BiCB-HexAB vector in 42-45 day-old GM2 mice (n=20) led to improved performance in the rotarod (FIG. 9A) and inverted screen (FIG. 9B) tests. Median survival of AAV treated GM2 mice was observed to be at least 265 days (accounting only for mice that have lived longer than 250 days), compared to 129.5 days for untreated GM2 mice (FIG. 9C; P<0.0001). No naïve GM2 mice were alive at 246 (n=7) and 299 (n=2) days of age (FIG. 9C). Data indicates a therapeutic benefit of AAV9-BiCB-HexAB vector delivered LIT in young adult mice.

SEQUENCES

>SEQ ID NO: 1; HexA amino acid sequence
MTSSRLWFSLLLAAAFAGRATALWPWPQNFQTSDQRYVLYPNNFQFQYDVSSAAQPG
CSVLDEAFQRYRDLLFGSGSWPRPYLTGKRHTLEKNVLVVSVVTPGCNQLPTLESVEN
YTLTINDDQCLLLSETVWGALRGLETFSQLVWKSAEGTFFINKTEIEDFPRFPHRGLLLD
TSRHYLPLSSILDTLDVMAYNKLNVFHWHLVDDPSFPYESFTFPELMRKGSYNPVTHIY
TAQDVKEVIEYARLRGIRVLAEFDTPGHTLSWGPGIPGLLTPCYSGSEPSGTFGPVNPSL
NNTYEFMSTFFLEVSSVFPDFYLHLGGDEVDFTCWKSNPEIQDFMRKKGFGEDFKQLES
FYIQTLLDIVSSYGKGYVVWQEVFDNKVKIQPDTIIQVWREDIPVNYMKELELVTKAGF
RALLSAPWYLNRISYGPDWKDFYVVEPLAFEGTPEQKALVIGGEACMWGEYVDNTNL
VPRLWPRAGAVAERLWSNKLTSDLTFAYERLSHFRCELLRRGVQAQPLNVGFCEQEFE
QT
>SEQ ID NO: 2; HexB amino acid sequence
MELCGLGLPRPPMLLALLLATLLAAMLALLTQVALVVQVAEAARAPSVSAKPGPALW
PLPLSVKMTPNLLHLAPENFYISHSPNSTAGPSCTLLEEAFRRYHGYIFGFYKWHHEPAE
FQAKTQVQQLLVSITLQSECDAFPNISSDESYTLLVKEPVAVLKANRVWGALRGLETFS
QLVYQDSYGTFTINESTIIDSPRFSHRGILIDTSRHYLPVKIILKTLDAMAFNKFNVLHWHI
VDDQSFPYQSITFPELSNKGSYSLSHVYTPNDVRMVIEYARLRGIRVLPEFDTPGHTLSW
GKGQKDLLTPCYSRQNKLDSFGPINPTLNTTYSFLTTFFKEISEVFPDQFIHLGGDEVEFK
CWESNPKIQDFMRQKGFGTDFKKLESFYIQKVLDIIATINKGSIVWQEVFDDKAKLAPG
TIVEVWKDSAYPEELSRVTASGFPVILSAPWYLDLISYGQDWRKYYKVEPLDFGGTQK
QKQLFIGGEACLWGEYVDATNLTPRLWPRASAVGERLWSSKDVRDMDDAYDRLTRH
RCRMVERGIAAQPLYAGYCNHENM
>SEQ ID NO: 3; AAV-BiCBA-HexAB nucleic acid sequence
TTGGCCACTCCCTCTCTGCGCGCTCGCTCGCTCACTGAGGCCGGGCGACCAAAGGTC
GCCCGACGCCCGGGCTTTGCCCGGGCGGCCTCAGTGAGCGAGCGAGCGCGCAGAGA
GGGAGTGGCCAACTCCATCACTAGGGGTTCCTAGATCTGAATTCTACCACATTTGTA
GAGGTTTTACTTGCTTTAAAAAACCTCCCACATCTCCCCCTGAACCTGAAACATAAA
ATGAATGCAATTGTTGTTGTTAACTTGTTTATTGCAGCTTATAATGGTTACAAATAA
AGCAATAGCATCACAAATTTCACAAATAAAGCATTTTTTTCACTGCATTCTAGTTGT
GGTTTGTCCAAACTCATCAATGTATCTTATCATGTCTGTCGAGGCGGCCGCTCAGGT
CTGCTCAAATTCCTGCTCACAGAATCCGACATTCAGGGGCTGTGCCTGCACGCCTCT
CCTCAGCAGCTCGCACCTGAAGTGGCTCAGCCTTTCGTAAGCGAATGTCAGATCAG
AGGTCAGCTTGTTGGACCACAGTCTCTCGGCCACTGCTCCGGCCCTGGGCCACAGTC
GTGGGACCAGATTGGTGTTGTCCACATACTCGCCCCACATACATGCTTCGCCCCCGA
TGACCAGAGCTTTCTGCTCAGGAGTCCCTTCAAAGGCCAGGGGCTCCACAATGTAG
AAATCCTTCCAGTCTGGTCCATATGAGATCCTGTTCAGGTACCAAGGGGCACTCAGC
AGTGCTCGGAAGCCAGCTTTTGTCACCAGTTCCAGCTCCTTCATATAATTGACAGGA
ATATCCTCGCGCCACACTTGAATGATGGTATCGGGCTGGATTTTGACCTTGTTGTCA
AACACTTCCTGCCAGACCACATAGCCCTTTCCGTATGAACTCACAATGTCCAGCAGA
GTCTGGATGTAGAAACTCTCCAGCTGCTTGAAGTCTTCGCCGAACCCTTTCTTCCGC
ATAAAGTCCTGGATCTCTGGATTAGACTTCCAACATGTGAAATCCACTTCGTCTCCG
CCCAGGTGCAGATAAAAGTCAGGGAAGACAGAGGACACCTCCAGAAAGAATGTAG
ACATGAACTCGTAGGTATTGTTCAGGGATGGGTTCACAGGTCCGAAGGTGCCGCTT
GGCTCACTCCCAGAGTAGCAAGGAGTCAGCAGTCCGGGAATTCCTGGGCCCCAGCT
CAGAGTATGGCCAGGTGTATCGAATTCAGCCAGCACTCGAATCCCCCTCAGTCGGG
CATACTCGATGACTTCTTTCACGTCCTGGGCGGTATAGATGTGAGTCACGGGATTGT
AGCTGCCCTTGCGCATCAGCTCTGGAAATGTGAAGCTTTCGTAAGGAAATGAGGGA
TCGTCGACCAGATGCCAGTGGAACACGTTCAGCTTGTTATAAGCCATCACATCCAG
AGTGTCCAGAATGCTTGACAGGGGCAGGTAATGTCTAGATGTATCCAGCAGCAGCC
CCCGGTGTGGAAATCTAGGGAAGTCTTCGATCTCAGTTTTGTTGATAAAGAATGTTC
CCTCGGCTGACTTCCAGACCAGCTGACTGAAAGTCTCCAGGCCCCTCAGTGCCCCCC
ACACTGTTTCGGACAGCAGCAGACACTGATCGTCATTGATTGTCAGGGTATAGTTCT
CCACGGATTCCAGGGTGGGCAGCTGATTGCAGCCTGGAGTGACCACGCTGACCACC
AGCACGTTTTTCTCCAGGGTGTGGCGCTTCCCAGTCAGATAGGGCCGTGGCCAGGA
CCCGCTTCCAAACAGCAGATCGCGGTACCGCTGGAATGCCTCGTCCAGGACGCTGC
ATCCTGGCTGAGCGGCGCTGCTCACGTCGTACTGAAACTGGAAATTGTTTGGATACA
GCACGTACCTCTGGTCAGATGTCTGGAAGTTCTGAGGCCATGGCCACAGGGCGGTT
GCTCGTCCAGCAAATGCAGCGGCCAGCAGCAGGGAGAACCACAGTCTGGAGCTGGT
CATGGTGGCTTTATCGATACTAGCGCTAGAGCTTGCTCCCGCCCGCCGCGCGCTTCG
CTTTTTATAGGGCCGCCGCCGCCGCCGCCTCGCCATAAAAGGAAACTTTCGGAGCG
CGCCGCTCTGATTGGCTGCCGCCGCACCTCTCCGCCTCGCCCCGCCCCGCCCCTCGC
CCCCATCGCTGCACAAAATAATTAAAAAATAAATAAATACAAAATTGGGGGTGGGG
AGGGGGGGGAGATGGGGAGAGTGAAGCAGAACGTGGCCTCGGATCCCCCGGGCTG
CAGTATTAATAGTAATCAATTACGGGGTCATTAGTTCATAGCCCATATATGGAGTTC
CGCGTTACATAACTTACGGTAAATGGCCCGCCTGGCTGACCGCCCAACGACCCCCG
CCCATTGACGTCAATAATGACGTATGTTCCCATAGTAACGCCAATAGGGACTTTCCA
TTGACGTCAATGGGTGGAGTATTTACGGTAAACTGCCCACTTGGCAGTACATCAAGT
GTATCATATGCCAAGTACGCCCCCTATTGACGTCAATGACGGTAAATGGCCCGCCTG
GCATTATGCCCAGTACATGACCTTATGGGACTTTCCTACTTGGCAGTACATCTACGT
ATTAGTCATCGCTATTACCATGTCGAGGCCACGTTCTGCTTCACTCTCCCCATCTCCC
CCCCCTCCCCACCCCCAATTTTGTATTTATTTATTTTTTAATTATTTTGTGCAGCGAT
GGGGGCGAGGGGCGGGGCGGGGCGAGGCGGAGAGGTGCGGCGGCAGCCAATCAG
AGCGGCGCGCTCCGAAAGTTTCCTTTTATGGCGAGGCGGCGGCGGCGGCGGCCCTA
TAAAAAGCGAAGCGCGCGGCGGGCGGGAGCAAGCTCGCTAGCACTAGTGGAGCAC
CATGGAGCTGTGCGGGCTGGGGCTGCCCCGGCCGCCCATGCTGCTGGCGCTGCTGTT
GGCGACACTGCTGGCGGCGATGTTGGCGCTGCTGACTCAGGTGGCGCTGGTGGTGC
AGGTGGCGGAGGCGGCTCGGGCCCCGAGCGTCTCGGCCAAGCCGGGGCCGGCGCT
GTGGCCCCTGCCGCTCTCGGTGAAGATGACCCCGAACCTGCTGCATCTCGCCCCGGA
GAACTTCTACATCAGCCACAGCCCCAATTCCACGGCGGGCCCCTCCTGCACCCTGCT
GGAGGAAGCGTTTCGACGATATCATGGCTATATTTTTGGTTTCTACAAGTGGCATCA
TGAACCTGCTGAATTCCAGGCTAAAACCCAGGTTCAGCAACTTCTTGTCTCAATCAC
CCTTCAGTCAGAGTGTGATGCTTTCCCCAACATATCTTCAGATGAGTCTTATACTTTA
CTTGTGAAAGAACCAGTGGCTGTCCTTAAGGCCAACAGAGTTTGGGGAGCATTACG
AGGTTTAGAGACCTTTAGCCAGTTAGTTTATCAAGATTCTTATGGAACTTTCACCAT
CAATGAATCCACCATTATTGATTCTCCAAGGTTTTCTCACAGAGGAATTTTGATTGA
TACATCCAGACATTATCTGCCAGTTAAGATTATTCTTAAAACTCTGGATGCCATGGC
TTTTAATAAGTTTAATGTTCTTCACTGGCACATAGTTGATGACCAGTCTTTCCCATAT
CAGAGCATCACTTTTCCTGAGTTAAGCAATAAAGGAAGCTATTCTTTGTCTCATGTT
TATACACCAAATGATGTCCGTATGGTGATTGAATATGCCAGATTACGAGGAATTCG
AGTCCTGCCAGAATTTGATACCCCTGGGCATACACTATCTTGGGGAAAAGGTCAGA
AAGACCTCCTGACTCCATGTTACAGTAGACAAAACAAGTTGGACTCTTTTGGACCTA
TAAACCCTACTCTGAATACAACATACAGCTTCCTTACTACATTTTTCAAAGAAATTA
GTGAGGTGTTTCCAGATCAATTCATTCATTTGGGAGGAGATGAAGTGGAATTTAAAT
GTTGGGAATCAAATCCAAAAATTCAAGATTTCATGAGGCAAAAAGGCTTTGGCACA
GATTTTAAGAAACTAGAATCTTTCTACATTCAAAAGGTTTTGGATATTATTGCAACC
ATAAACAAGGGATCCATTGTCTGGCAGGAGGTTTTTGATGATAAAGCAAAGCTTGC
GCCGGGCACAATAGTTGAAGTATGGAAAGACAGCGCATATCCTGAGGAACTCAGTA
GAGTCACAGCATCTGGCTTCCCTGTAATCCTTTCTGCTCCTTGGTACTTAGATTTGAT
TAGCTATGGACAAGATTGGAGGAAATACTATAAAGTGGAACCTCTTGATTTTGGCG
GTACTCAGAAACAGAAACAACTTTTCATTGGTGGAGAAGCTTGTCTATGGGGAGAA
TATGTGGATGCAACTAACCTCACTCCAAGATTATGGCCTCGGGCAAGTGCTGTTGGT
GAGAGACTCTGGAGTTCCAAAGATGTCAGAGATATGGATGACGCCTATGACAGACT
GACAAGGCACCGCTGCAGGATGGTCGAACGTGGAATAGCTGCACAACCTCTTTATG
CTGGATATTGTAACCATGAGAACATGTAACTCGAGGCGGCCGCTCTAGAGATCTTTT
TCCCTCTGCCAAAAATTATGGGGACATCATGAAGCCCCTTGAGCATCTGACTTCTGG
CTAATAAAGGAAATTTATTTTCATTGCAATAGTGTGTTGGAATTTTTTGTGTCTCTCA
CTCGGCATGCTGGGGAGAGATCTAGGAACCCCTAGTGATGGAGTTGGCCACTCCCT
CTCTGCGCGCTCGCTCGCTCACTGAGGCCGCCCGGGCAAAGCCCGGGCGTCGGGCG
ACCTTTGGTCGCCCGGCCTCAGTGAGCGAGCGAGCGCGCAGAGAGGGAGTGGCCAT
GCAGCCAGCTGGCGTAATAGCGAAGAGGCCCGCACCGATCGCCCTTCCCAACAGTT
GCGTAGCCTGAATGGCGAATGGCGCGACGCGCCCTGTAGCGGCGCATTAAGCGCGG
CGGGTGTGGTGGTTACGCGCAGCGTGACCGCTACACTTGCCAGCGCCCTAGCGCCC
GCTCCTTTCGCTTTCTTCCCTTCCTTTCTCGCCACGTTCGCCGGCTTTCCCCGTCAAG
CTCTAAATCGGGGGCTCCCTTTAGGGTTCCGATTTAGTGCTTTACGGCACCTCGACC
CCAAAAAACTTGATTAGGGTGATGGTTCACGTAGTGGGCCATCGCCCTGATAGACG
GTTTTTCGCCCTTTGACGTTGGAGTCCACGTTCTTTAATAGTGGACTCTTGTTCCAAA
CTGGAACAACACTCAACCCTATCTCGGTCTATTCTTTTGATTTATAAGGGATTTTGCC
GATTTCGGCCTATTGGTTAAAAAATGAGCTGATTTAACAAAAATTTAACGCGAATTT
TAACAAAATATTAACGTTTACAATTTCCTGATGCGGTATTTTCTCCTTACGCATCTGT
GCGGTATTTCACACCGCATATGGTGCACTCTCAGTACAATCTGCTCTGATGCCGCAT
AGTTAAGCCAGCCCCGACACCCGCCAACACCCGCTGACGCGCCCTGACGGGCTTGT
CTGCTCCCGGCATCCGCTTACAGACAAGCTGTGACCGTCTCCGGGAGCTGCATGTGT
CAGAGGTTTTCACCGTCATCACCGAAACGCGCGAGACGAAAGGGCCTCGTGATACG
CCTATTTTTATAGGTTAATGTCATGATAATAATGGTTTCTTAGACGTCAGGTGGCAC
TTTTCGGGGAAATGTGCGCGGAACCCCTATTTGTTTATTTTTCTAAATACATTCAAAT
ATGTATCCGCTCATGAGACAATAACCCTGATAAATGCTTCAATAATATTGAAAAAG
GAAGAGTATGAGTATTCAACATTTCCGTGTCGCCCTTATTCCCTTTTTTGCGGCATTT
TGCCTTCCTGTTTTTGCTCACCCAGAAACGCTGGTGAAAGTAAAAGATGCTGAAGAT
CAGTTGGGTGCACGAGTGGGTTACATCGAACTGGATCTCAACAGCGGTAAGATCCT
TGAGAGTTTTCGCCCCGAAGAACGTTTTCCAATGATGAGCACTTTTAAAGTTCTGCT
CTATTCTCAGAATGACTTGGTTGAGTACTCACCAGTCACAGAAAAGCATCTTACGGA
TGGCATGACGTAAGAGAATTATGCAGTGCTGCCATAACCATGAGTGATAACACTGC
GGCCAACTTACTTCTGACAACGATCGGAGGACCGAAGGAGCTAACCGCTTTTTTGC
ACAACATGGGGGATCATGTAACTCGCCTTGATCGTTGGGAACCGGAGCTGAATGAA
GCCATACCAAACGACGAGCGTGACACCACGATGCCTGTAGCAATGGCAACAACGTT
GCGCAAACTATTAACTGGCGAACTACTTACTCTAGCTTCCCGGCAACAATTAATAGA
CTGGATGGAGGCGGATAAAGTTGCAGGACCACTTCTGCGCTCGGCCCTTCCGGCTG
GCTGGTTTATTGCTGATAAATCTGGAGCCGGTGAGCGTGGGTCTCGCGGTATCATTG
CAGCACTGGGGCCAGATGGTAAGCCCTCCCGTATCGTAGTTATCTACACGACGGGG
AGTCAGGCAACTATGGATGAACGAAATAGACAGATCGCTGAGATAGGTGCCTCACT
GATTAAGCATTGGTAACTGTCAGACCAAGTTTACTCATATATACTTTAGATTGATTT
AAAACTTCATTTTTAATTTAAAAGGATCTAGGTGAAGATCCTTTTTGATAATCTCAT
GACCAAAATCCCTTAACGTGAGTTTTCGTTCCACTGAGCGTCAGACCCCGTAGAAA
AGATCAAAGGATCTTCTTGAGATCCTTTTTTTCTGCGCGTAATCTGCTGCTTGCAAA
CAAAAAAACCACCGCTACCAGCGGTGGTTTGTTTGCCGGATCAAGAGCTACCAACT
CTTTTTCCGAAGGTAACTGGCTTCAGCAGAGCGCAGATACCAAATACTGTCCTTCTA
GTGTAGCCGTAGTTAGGCCACCACTTCAAGAACTCTGTAGCACCGCCTACATACCTC
GCTCTGCTAATCCTGTTACCAGTGGCTGCTGCCAGTGGCGATAAGTCGTGTCTTACC
GGGTTGGACTCAAGACGATAGTTACCGGATAAGGCGCAGCGGTCGGGCTGAACGG
GGGGTTCGTGCACACAGCCCAGCTTGGAGCGAACGACCTACACCGAACTGAGATAC
CTACAGCGTGAGCATTGAGAAAGCGCCACGCTTCCCGAAGGGAGAAAGGCGGACA
GGTATCCGGTAAGCGGCAGGGTCGGAACAGGAGAGCGCACGAGGGAGCTTCCAGG
GGGAAACGCCTGGTATCTTTATAGTCCTGTCGGGTTTCGCCACCTCTGACTTGAGCG
TCGATTTTTGTGATGCTCGTCAGGGGGGCGGAGCCTATGGAAAAACGCCAGCAACG
CGGCCTTTTTACGGTTCCTGGCCTTTTGCTGGCCTTTTGCTCACATGTTCTTTCCTGC
GTTATCCCCTGATTCTGTGGATAACCGTATTACCGCCTTTGAGTGAGCTGATACCGC
TCGCCGCAGCCGAACGACCGAGCGCAGCGAGTCAGTGAGCGAGGAAGCGGAAGAG
CGCCCAATACGCAAACCGCCTCTCCCCGCGCGTTGGCCGATTCATTAATGCAGCTGG
GCTGCAGGGGGGGGGGGGGGGGGGTGGGGGGGGGGGGGGGGGG
>SEQ ID NO: 4; AAV-BiCBA-HexA:codon optimized-HexB nucleic acid
sequence
TTGGCCACTCCCTCTCTGCGCGCTCGCTCGCTCACTGAGGCCGGGCGACCAAAGGTC
GCCCGACGCCCGGGCTTTGCCCGGGCGGCCTCAGTGAGCGAGCGAGCGCGCAGAGA
GGGAGTGGCCAACTCCATCACTAGGGGTTCCTAGATCTGAATTCTACCACATTTGTA
GAGGTTTTACTTGCTTTAAAAAACCTCCCACATCTCCCCCTGAACCTGAAACATAAA
ATGAATGCAATTGTTGTTGTTAACTTGTTTATTGCAGCTTATAATGGTTACAAATAA
AGCAATAGCATCACAAATTTCACAAATAAAGCATTTTTTTCACTGCATTCTAGTTGT
GGTTTGTCCAAACTCATCAATGTATCTTATCATGTCTGTCGAGGCGGCCGCTCAGGT
CTGCTCAAATTCCTGCTCACAGAATCCGACATTCAGGGGCTGTGCCTGCACGCCTCT
CCTCAGCAGCTCGCACCTGAAGTGGCTCAGCCTTTCGTAAGCGAATGTCAGATCAG
AGGTCAGCTTGTTGGACCACAGTCTCTCGGCCACTGCTCCGGCCCTGGGCCACAGTC
GTGGGACCAGATTGGTGTTGTCCACATACTCGCCCCACATACATGCTTCGCCCCCGA
TGACCAGAGCTTTCTGCTCAGGAGTCCCTTCAAAGGCCAGGGGCTCCACAATGTAG
AAATCCTTCCAGTCTGGTCCATATGAGATCCTGTTCAGGTACCAAGGGGCACTCAGC
AGTGCTCGGAAGCCAGCTTTTGTCACCAGTTCCAGCTCCTTCATATAATTGACAGGA
ATATCCTCGCGCCACACTTGAATGATGGTATCGGGCTGGATTTTGACCTTGTTGTCA
AACACTTCCTGCCAGACCACATAGCCCTTTCCGTATGAACTCACAATGTCCAGCAGA
GTCTGGATGTAGAAACTCTCCAGCTGCTTGAAGTCTTCGCCGAACCCTTTCTTCCGC
ATAAAGTCCTGGATCTCTGGATTAGACTTCCAACATGTGAAATCCACTTCGTCTCCG
CCCAGGTGCAGATAAAAGTCAGGGAAGACAGAGGACACCTCCAGAAAGAATGTAG
ACATGAACTCGTAGGTATTGTTCAGGGATGGGTTCACAGGTCCGAAGGTGCCGCTT
GGCTCACTCCCAGAGTAGCAAGGAGTCAGCAGTCCGGGAATTCCTGGGCCCCAGCT
CAGAGTATGGCCAGGTGTATCGAATTCAGCCAGCACTCGAATCCCCCTCAGTCGGG
CATACTCGATGACTTCTTTCACGTCCTGGGCGGTATAGATGTGAGTCACGGGATTGT
AGCTGCCCTTGCGCATCAGCTCTGGAAATGTGAAGCTTTCGTAAGGAAATGAGGGA
TCGTCGACCAGATGCCAGTGGAACACGTTCAGCTTGTTATAAGCCATCACATCCAG
AGTGTCCAGAATGCTTGACAGGGGCAGGTAATGTCTAGATGTATCCAGCAGCAGCC
CCCGGTGTGGAAATCTAGGGAAGTCTTCGATCTCAGTTTTGTTGATAAAGAATGTTC
CCTCGGCTGACTTCCAGACCAGCTGACTGAAAGTCTCCAGGCCCCTCAGTGCCCCCC
ACACTGTTTCGGACAGCAGCAGACACTGATCGTCATTGATTGTCAGGGTATAGTTCT
CCACGGATTCCAGGGTGGGCAGCTGATTGCAGCCTGGAGTGACCACGCTGACCACC
AGCACGTTTTTCTCCAGGGTGTGGCGCTTCCCAGTCAGATAGGGCCGTGGCCAGGA
CCCGCTTCCAAACAGCAGATCGCGGTACCGCTGGAATGCCTCGTCCAGGACGCTGC
ATCCTGGCTGAGCGGCGCTGCTCACGTCGTACTGAAACTGGAAATTGTTTGGATACA
GCACGTACCTCTGGTCAGATGTCTGGAAGTTCTGAGGCCATGGCCACAGGGCGGTT
GCTCGTCCAGCAAATGCAGCGGCCAGCAGCAGGGAGAACCACAGTCTGGAGCTGGT
CATGGTGGCTTTATCGATACTAGCGCTAGAGCTTGCTCCCGCCCGCCGCGCGCTTCG
CTTTTTATAGGGCCGCCGCCGCCGCCGCCTCGCCATAAAAGGAAACTTTCGGAGCG
CGCCGCTCTGATTGGCTGCCGCCGCACCTCTCCGCCTCGCCCCGCCCCGCCCCTCGC
CCCCATCGCTGCACAAAATAATTAAAAAATAAATAAATACAAAATTGGGGGTGGGG
AGGGGGGGGAGATGGGGAGAGTGAAGCAGAACGTGGCCTCGGATCCCCCGGGCTG
CAGTATTAATAGTAATCAATTACGGGGTCATTAGTTCATAGCCCATATATGGAGTTC
CGCGTTACATAACTTACGGTAAATGGCCCGCCTGGCTGACCGCCCAACGACCCCCG
CCCATTGACGTCAATAATGACGTATGTTCCCATAGTAACGCCAATAGGGACTTTCCA
TTGACGTCAATGGGTGGAGTATTTACGGTAAACTGCCCACTTGGCAGTACATCAAGT
GTATCATATGCCAAGTACGCCCCCTATTGACGTCAATGACGGTAAATGGCCCGCCTG
GCATTATGCCCAGTACATGACCTTATGGGACTTTCCTACTTGGCAGTACATCTACGT
ATTAGTCATCGCTATTACCATGTCGAGGCCACGTTCTGCTTCACTCTCCCCATCTCCC
CCCCCTCCCCACCCCCAATTTTGTATTTATTTATTTTTTAATTATTTTGTGCAGCGAT
GGGGGCGAGGGGCGGGGCGGGGCGAGGCGGAGAGGTGCGGCGGCAGCCAATCAG
AGCGGCGCGCTCCGAAAGTTTCCTTTTATGGCGAGGCGGCGGCGGCGGCGGCCCTA
TAAAAAGCGAAGCGCGCGGCGGGCGGGAGCAAGCTCGCTAGCACTAGTGGAGCAC
CATGGAGCTGTGCGGGCTGGGGCTGCCCCGGCCGCCCATGCTGCTGGCGCTGCTGTT
GGCGACACTGCTGGCGGCGATGTTGGCGCTGCTGACTCAGGTGGCGCTGGTGGTGC
AGGTGGCGGAGGCGGCTCGGGCCCCGAGCGTCTCGGCCAAGCCGGGGCCGGCGCT
GTGGCCCCTGCCGCTCTCGGTGAAGATGACCCCGAACCTGCTGCATCTCGCCCCGGA
GAACTTCTACATCAGCCACAGCCCCAATTCCACGGCGGGCCCCTCCTGCACCCTGCT
GGAGGAAGCGTTTCGACGATATCATGGCTATATTTTTGGTTTCTACAAGTGGCATCA
TGAACCTGCTGAATTCCAGGCTAAAACCCAGGTTCAGCAACTTCTTGTCTCAATCAC
CCTTCAGTCAGAGTGTGATGCTTTCCCCAACATATCTTCAGATGAGTCTTATACTTTA
CTTGTGAAAGAACCAGTGGCTGTCCTTAAGGCCAACAGAGTTTGGGGAGCATTACG
AGGTTTAGAGACCTTTAGCCAGTTAGTTTATCAAGATTCTTATGGAACTTTCACCAT
CAATGAATCCACCATTATTGATTCTCCAAGGTTTTCTCACAGAGGAATTTTGATTGA
TACATCCAGACATTATCTGCCAGTTAAGATTATTCTTAAAACTCTGGATGCCATGGC
TTTTAATAAGTTTAATGTTCTTCACTGGCACATAGTTGATGACCAGTCTTTCCCATAT
CAGAGCATCACTTTTCCTGAGTTAAGCAATAAAGGAAGCTATTCTTTGTCTCATGTT
TATACACCAAATGATGTCCGTATGGTGATTGAATATGCCAGATTACGAGGAATTCG
AGTCCTGCCAGAATTTGATACCCCTGGGCATACACTATCTTGGGGAAAAGGTCAGA
AAGACCTCCTGACTCCATGTTACAGTAGACAAAACAAGTTGGACTCTTTTGGACCTA
TAAACCCTACTCTGAATACAACATACAGCTTCCTTACTACATTTTTCAAAGAAATTA
GTGAGGTGTTTCCAGATCAATTCATTCATTTGGGAGGAGATGAAGTGGAATTTAAAT
GTTGGGAATCAAATCCAAAAATTCAAGATTTCATGAGGCAAAAAGGCTTTGGCACA
GATTTTAAGAAACTAGAATCTTTCTACATTCAAAAGGTTTTGGATATTATTGCAACC
ATAAACAAGGGATCCATTGTCTGGCAGGAGGTTTTTGATGATAAAGCAAAGCTTGC
GCCGGGCACAATAGTTGAAGTATGGAAAGACAGCGCATATCCTGAGGAACTCAGTA
GAGTCACAGCATCTGGCTTCCCTGTAATCCTTTCTGCTCCTTGGTACTTAGATTTGAT
TAGCTATGGACAAGATTGGAGGAAATACTATAAAGTGGAACCTCTTGATTTTGGCG
GTACTCAGAAACAGAAACAACTTTTCATTGGTGGAGAAGCTTGTCTATGGGGAGAA
TATGTGGATGCAACTAACCTCACTCCAAGATTATGGCCTCGGGCAAGTGCTGTTGGT
GAGAGACTCTGGAGTTCCAAAGATGTCAGAGATATGGATGACGCCTATGACAGACT
GACAAGGCACCGCTGCAGGATGGTCGAACGTGGAATAGCTGCACAACCTCTTTATG
CTGGATATTGTAACCATGAGAACATGTAACTCGAGGCGGCCGCTCTAGAGATCTTTT
TCCCTCTGCCAAAAATTATGGGGACATCATGAAGCCCCTTGAGCATCTGACTTCTGG
CTAATAAAGGAAATTTATTTTCATTGCAATAGTGTGTTGGAATTTTTTGTGTCTCTCA
CTCGGCATGCTGGGGAGAGATCTAGGAACCCCTAGTGATGGAGTTGGCCACTCCCT
CTCTGCGCGCTCGCTCGCTCACTGAGGCCGCCCGGGCAAAGCCCGGGCGTCGGGCG
ACCTTTGGTCGCCCGGCCTCAGTGAGCGAGCGAGCGCGCAGAGAGGGAGTGGCCAT
GCAGCCAGCTGGCGTAATAGCGAAGAGGCCCGCACCGATCGCCCTTCCCAACAGTT
GCGTAGCCTGAATGGCGAATGGCGCGACGCGCCCTGTAGCGGCGCATTAAGCGCGG
CGGGTGTGGTGGTTACGCGCAGCGTGACCGCTACACTTGCCAGCGCCCTAGCGCCC
GCTCCTTTCGCTTTCTTCCCTTCCTTTCTCGCCACGTTCGCCGGCTTTCCCCGTCAAG
CTCTAAATCGGGGGCTCCCTTTAGGGTTCCGATTTAGTGCTTTACGGCACCTCGACC
CCAAAAAACTTGATTAGGGTGATGGTTCACGTAGTGGGCCATCGCCCTGATAGACG
GTTTTTCGCCCTTTGACGTTGGAGTCCACGTTCTTTAATAGTGGACTCTTGTTCCAAA
CTGGAACAACACTCAACCCTATCTCGGTCTATTCTTTTGATTTATAAGGGATTTTGCC
GATTTCGGCCTATTGGTTAAAAAATGAGCTGATTTAACAAAAATTTAACGCGAATTT
TAACAAAATATTAACGTTTACAATTTCCTGATGCGGTATTTTCTCCTTACGCATCTGT
GCGGTATTTCACACCGCATATGGTGCACTCTCAGTACAATCTGCTCTGATGCCGCAT
AGTTAAGCCAGCCCCGACACCCGCCAACACCCGCTGACGCGCCCTGACGGGCTTGT
CTGCTCCCGGCATCCGCTTACAGACAAGCTGTGACCGTCTCCGGGAGCTGCATGTGT
CAGAGGTTTTCACCGTCATCACCGAAACGCGCGAGACGAAAGGGCCTCGTGATACG
CCTATTTTTATAGGTTAATGTCATGATAATAATGGTTTCTTAGACGTCAGGTGGCAC
TTTTCGGGGAAATGTGCGCGGAACCCCTATTTGTTTATTTTTCTAAATACATTCAAAT
ATGTATCCGCTCATGAGACAATAACCCTGATAAATGCTTCAATAATATTGAAAAAG
GAAGAGTATGAGTATTCAACATTTCCGTGTCGCCCTTATTCCCTTTTTTGCGGCATTT
TGCCTTCCTGTTTTTGCTCACCCAGAAACGCTGGTGAAAGTAAAAGATGCTGAAGAT
CAGTTGGGTGCACGAGTGGGTTACATCGAACTGGATCTCAACAGCGGTAAGATCCT
TGAGAGTTTTCGCCCCGAAGAACGTTTTCCAATGATGAGCACTTTTAAAGTTCTGCT
ATGTGGCGCGGTATTATCCCGTATTGACGCCGGGCAAGAGCAACTCGGTCGCCGCA
TACACTATTCAGAATGACTTGGTGAGTACTCACCAGTCACAGAAAAGCATCTTACG
GATGGCATGACAGTAAGAGAATTATGCAGTGCTGCCATAACCATGAGTGATAACAC
TGCGGCCAACTTACTTCTGACAACGATCGGAGGACCGAAGGAGCTAACCGCTTTTTT
GCACAACATGGGGGATCATGTAACTCGCCTTGATCGTTGGGAACCGGAGCTGAATG
AAGCCATACCAAACGACGAGCGTGACACCACGATGCCTGTAGCAATGGCAACAAC
GTTGCGCAAACTATTAACTGGCGAACTACTTACTCTAGCTTCCCGGCAACAATTAAT
AGACTGGATGGAGGCGGATAAAGTTGCAGGACCACTTCTGCGCTCGGCCCTTCCGG
CTGGCTGGTTTATTGCTGATAAATCTGGAGCCGGTGAGCGTGGGTCTCGCGGTATCA
TTGCAGCACTGGGGCCAGATGGTAAGCCCTCCCGTATCGTAGTTATCTACACGACG
GGGAGTCAGGCAACTATGGATGAACGAAATAGACAGATCGCTGAGATAGGTGCCTC
ACTGATTAAGCATTGGTAACTGTCAGACCAAGTTTACTCATATATACTTTAGATTGA
TTTAAAACTTCATTTTTAATTTAAAAGGATCTAGGTGAGATCCTTTTTGATAATCTCA
TGACCAAAATCCCTTAACGTGAGTTTTCGTTCCACTGAGCGTCAGACCCCGTAGAAA
AGATCAAAGGATCTTCTTGAGATCCTTTTTTTCTGCGCGTAATCTGCTGCTTGCAAA
CAAAAAAACCACCGCTACCAGCGGTGGTTTGTTTGCCGGATCAAGAGCTACCAACT
CTTTTTCCGAAGGTAACTGGCTTCAGCAGAGCGCAGATACCAAATACTGTCCTTCTA
GTGTAGCCGTAGTTAGGCCACCACTTCAAGAACTCTGTAGCACCGCCTACATACCTC
GCTCTGCTAATCCTGTTACCAGTGGCTGCTGCCAGTGGCGATAAGTCGTGTCTTACC
GGGTTGGACTCAAGACGATAGTTACCGGATAAGGCGCAGCGGTCGGGCTGAACGG
GGGGTTCGCATTGAGAAAGCGCCACGCTTCCCGAAGGGAGAAAGGCGGACAGGTA
TCCGGTAAGCGGCAGGGTCGGAACAGGAGAGCGCACGAGGGAGCTTCCAGGGGGA
AACGCCTGGTATCTTTATAGTCCTGTCGGGTTTCGCCACCTCTGACTTGAGCGTCGA
TTTTTGTGATGCTCGTCAGGGGGGCGGAGCCTATGGAAAAACGCCAGCAACGCGGC
CTTTTACGGTTCCTGGCCTTTTGCTGGCCTTTTGCTCACATGTTCTTTCCTGCGTTATC
CCCTGATTCTGTGGATAACCGTATTACCGCCTTTGAGTGAGCTGATACCGCTCGCCG
CAGCCGAACGACCGAGCGCAGCGAGTCAGTGAGCGAGGAAGCGGAAGAGCGCCCA
ATACGCAAACCGCCTCTCCCCGCGCGTTGGCCGATTCATTATGCAGCTGGGCTGCAG
GGGGGGGGGGGGGGGGGTGGGGGGGGGGGGGGGG
GG
>SEQ ID NO: 5; AAV-BiCBA-HexA:codon optimized-HexB:codon
optimized nucleic acid sequence
TTGGCCACTCCCTCTCTGCGCGCTCGCTCGCTCACTGAGGCCGGGCGACCAAAGGTC
GCCCGACGCCCGGGCTTTGCCCGGGCGGCCTCAGTGAGCGAGCGAGCGCGCAGAGA
GGGAGTGGCCAACTCCATCACTAGGGGTTCCTAGATCTGAATTCTACCACATTTGTA
GAGGTTTTACTTGCTTTAAAAAACCTCCCACATCTCCCCCTGAACCTGAAACATAAA
ATGAATGCAATTGTTGTTGTTAACTTGTTTATTGCAGCTTATAATGGTTACAAATAA
AGCAATAGCATCACAAATTTCACAAATAAAGCATTTTTTTCACTGCATTCTAGTTGT
GGTTTGTCCAAACTCATCAATGTATCTTATCATGTCTGTCGAGGCGGCCGCTCAGGT
CTGCTCAAATTCCTGCTCACAGAATCCGACATTCAGGGGCTGTGCCTGCACGCCTCT
CCTCAGCAGCTCGCACCTGAAGTGGCTCAGCCTTTCGTAAGCGAATGTCAGATCAG
AGGTCAGCTTGTTGGACCACAGTCTCTCGGCCACTGCTCCGGCCCTGGGCCACAGTC
GTGGGACCAGATTGGTGTTGTCCACATACTCGCCCCACATACATGCTTCGCCCCCGA
TGACCAGAGCTTTCTGCTCAGGAGTCCCTTCAAAGGCCAGGGGCTCCACAATGTAG
AAATCCTTCCAGTCTGGTCCATATGAGATCCTGTTCAGGTACCAAGGGGCACTCAGC
AGTGCTCGGAAGCCAGCTTTTGTCACCAGTTCCAGCTCCTTCATATAATTGACAGGA
ATATCCTCGCGCCACACTTGAATGATGGTATCGGGCTGGATTTTGACCTTGTTGTCA
AACACTTCCTGCCAGACCACATAGCCCTTTCCGTATGAACTCACAATGTCCAGCAGA
GTCTGGATGTAGAAACTCTCCAGCTGCTTGAAGTCTTCGCCGAACCCTTTCTTCCGC
ATAAAGTCCTGGATCTCTGGATTAGACTTCCAACATGTGAAATCCACTTCGTCTCCG
CCCAGGTGCAGATAAAAGTCAGGGAAGACAGAGGACACCTCCAGAAAGAATGTAG
ACATGAACTCGTAGGTATTGTTCAGGGATGGGTTCACAGGTCCGAAGGTGCCGCTT
GGCTCACTCCCAGAGTAGCAAGGAGTCAGCAGTCCGGGAATTCCTGGGCCCCAGCT
CAGAGTATGGCCAGGTGTATCGAATTCAGCCAGCACTCGAATCCCCCTCAGTCGGG
CATACTCGATGACTTCTTTCACGTCCTGGGCGGTATAGATGTGAGTCACGGGATTGT
AGCTGCCCTTGCGCATCAGCTCTGGAAATGTGAAGCTTTCGTAAGGAAATGAGGGA
TCGTCGACCAGATGCCAGTGGAACACGTTCAGCTTGTTATAAGCCATCACATCCAG
AGTGTCCAGAATGCTTGACAGGGGCAGGTAATGTCTAGATGTATCCAGCAGCAGCC
CCCGGTGTGGAAATCTAGGGAAGTCTTCGATCTCAGTTTTGTTGATAAAGAATGTTC
CCTCGGCTGACTTCCAGACCAGCTGACTGAAAGTCTCCAGGCCCCTCAGTGCCCCCC
ACACTGTTTCGGACAGCAGCAGACACTGATCGTCATTGATTGTCAGGGTATAGTTCT
CCACGGATTCCAGGGTGGGCAGCTGATTGCAGCCTGGAGTGACCACGCTGACCACC
AGCACGTTTTTCTCCAGGGTGTGGCGCTTCCCAGTCAGATAGGGCCGTGGCCAGGA
CCCGCTTCCAAACAGCAGATCGCGGTACCGCTGGAATGCCTCGTCCAGGACGCTGC
ATCCTGGCTGAGCGGCGCTGCTCACGTCGTACTGAAACTGGAAATTGTTTGGATACA
GCACGTACCTCTGGTCAGATGTCTGGAAGTTCTGAGGCCATGGCCACAGGGCGGTT
GCTCGTCCAGCAAATGCAGCGGCCAGCAGCAGGGAGAACCACAGTCTGGAGCTGGT
CATGGTGGCTTTATCGATACTAGCGCTAGAGCTTGCTCCCGCCCGCCGCGCGCTTCG
CTTTTTATAGGGCCGCCGCCGCCGCCGCCTCGCCATAAAAGGAAACTTTCGGAGCG
CGCCGCTCTGATTGGCTGCCGCCGCACCTCTCCGCCTCGCCCCGCCCCGCCCCTCGC
CCCCATCGCTGCACAAAATAATTAAAAAATAAATAAATACAAAATTGGGGGTGGGG
AGGGGGGGGAGATGGGGAGAGTGAAGCAGAACGTGGCCTCGGATCCCCCGGGCTG
CAGTATTAATAGTAATCAATTACGGGGTCATTAGTTCATAGCCCATATATGGAGTTC
CGCGTTACATAACTTACGGTAAATGGCCCGCCTGGCTGACCGCCCAACGACCCCCG
CCCATTGACGTCAATAATGACGTATGTTCCCATAGTAACGCCAATAGGGACTTTCCA
TTGACGTCAATGGGTGGAGTATTTACGGTAAACTGCCCACTTGGCAGTACATCAAGT
GTATCATATGCCAAGTACGCCCCCTATTGACGTCAATGACGGTAAATGGCCCGCCTG
GCATTATGCCCAGTACATGACCTTATGGGACTTTCCTACTTGGCAGTACATCTACGT
ATTAGTCATCGCTATTACCATGTCGAGGCCACGTTCTGCTTCACTCTCCCCATCTCCC
CCCCCTCCCCACCCCCAATTTTGTATTTATTTATTTTTTAATTATTTTGTGCAGCGAT
GGGGGCGAGGGGCGGGGCGGGGCGAGGCGGAGAGGTGCGGCGGCAGCCAATCAG
AGCGGCGCGCTCCGAAAGTTTCCTTTTATGGCGAGGCGGCGGCGGCGGCGGCCCTA
TAAAAAGCGAAGCGCGCGGCGGGCGGGAGCAAGCTCGCTAGCACTAGTAAAGCCA
CCATGGAACTGTGCGGACTGGGACTGCCTAGACCCCCTATGCTGCTGGCTCTGCTGC
TGGCTACCCTGCTGGCTGCTATGCTGGCCCTGCTGACTCAGGTGGCCCTGGTGGTCC
AGGTCGCAGAGGCAGCTAGGGCTCCTTCAGTGAGCGCAAAGCCAGGACCTGCCCTG
TGGCCACTGCCACTGTCCGTGAAAATGACACCAAACCTGCTGCACCTGGCCCCCGA
AAATTTCTACATCTCCCATTCTCCTAACAGTACAGCAGGGCCATCATGCACTCTGCT
GGAGGAAGCCTTCCGGAGATACCACGGGTATATTTTCGGCTTCTACAAGTGGCACC
ATGAGCCCGCAGAATTTCAGGCCAAAACCCAGGTGCAGCAGCTGCTGGTCAGCATC
ACACTGCAGTCCGAGTGTGACGCTTTCCCAAATATTAGCTCCGATGAGAGCTACACC
CTGCTGGTGAAGGAACCCGTGGCTGTCCTGAAAGCAAACAGAGTCTGGGGCGCACT
GAGGGGGCTGGAGACATTCTCCCAGCTGGTGTACCAGGACTCTTACGGAACATTTA
CTATCAACGAATCCACTATCATCGACTCCCCTAGGTTTTCTCACCGCGGCATCCTGA
TTGATACTTCTCGCCATTACCTGCCAGTGAAGATCATTCTGAAAACCCTGGATGCAA
TGGCCTTCAACAAGTTTAATGTGCTGCACTGGCATATCGTCGACGATCAGTCTTTCC
CTTACCAGAGTATTACATTTCCAGAGCTGAGTAATAAAGGCAGTTACTCACTGAGCC
ACGTGTATACTCCAAACGACGTGAGAATGGTCATCGAGTATGCCAGGCTGCGCGGA
ATTAGGGTGCTGCCAGAATTCGATACTCCCGGCCATACCCTGTCTTGGGGCAAGGG
GCAGAAAGACCTGCTGACCCCCTGCTACAGTCGGCAGAACAAGCTGGATTCATTCG
GCCCTATCAACCCAACACTGAATACCACATACTCTTTTCTGACTACCTTCTTTAAGG
AGATCAGTGAAGTGTTCCCCGACCAGTTTATTCACCTGGGCGGGGATGAGGTCGAA
TTCAAGTGTTGGGAGAGCAATCCTAAAATCCAGGACTTTATGAGACAGAAGGGATT
CGGCACCGACTTCAAGAAACTGGAATCATTCTACATTCAGAAGGTGCTGGACATCA
TTGCCACAATCAACAAAGGGAGCATCGTGTGGCAGGAGGTCTTTGACGATAAGGCC
AAACTGGCTCCCGGAACTATCGTGGAAGTCTGGAAGGATAGCGCCTATCCTGAGGA
ACTGTCACGCGTGACCGCTAGCGGCTTTCCCGTCATCCTGAGCGCACCTTGGTACCT
GGACCTGATTTCCTATGGGCAGGATTGGCGAAAGTACTATAAAGTGGAGCCCCTGG
ACTTCGGAGGCACCCAGAAGCAGAAACAGCTGTTTATCGGGGGAGAGGCCTGCCTG
TGGGGGGAATACGTGGATGCTACCAATCTGACACCACGACTGTGGCCTCGGGCTAG
CGCAGTGGGAGAGCGCCTGTGGTCTAGTAAGGACGTGCGGGATATGGACGATGCCT
ATGACCGGCTGACCCGACATCGGTGTAGAATGGTGGAACGGGGCATTGCCGCTCAG
CCACTGTATGCCGGGTACTGTAATCACGAAAATATGTGACTCGAGGCGGCCGCTCT
AGAGATCTTTTTCCCTCTGCCAAAAATTATGGGGACATCATGAAGCCCCTTGAGCAT
CTGACTTCTGGCTAATAAAGGAAATTTATTTTCATTGCAATAGTGTGTTGGAATTTTT
TGTGTCTCTCACTCGGCATGCTGGGGAGAGATCTAGGAACCCCTAGTGATGGAGTT
GGCCACTCCCTCTCTGCGCGCTCGCTCGCTCACTGAGGCCGCCCGGGCAAAGCCCG
GGCGTCGGGCGACCTTTGGTCGCCCGGCCTCAGTGAGCGAGCGAGCGCGCAGAGAG
GGAGTGGCCATGCAGCCAGCTGGCGTAATAGCGAAGAGGCCCGCACCGATCGCCCT
TCCCAACAGTTGCGTAGCCTGAATGGCGAATGGCGCGACGCGCCCTGTAGCGGCGC
ATTAAGCGCGGCGGGTGTGGTGGTTACGCGCAGCGTGACCGCTACACTTGCCAGCG
CCCTAGCGCCCGCTCCTTTCGCTTTCTTCCCTTCCTTTCTCGCCACGTTCGCCGGCTTT
CCCCGTCAAGCTCTAAATCGGGGGCTCCCTTTAGGGTTCCGATTTAGTGCTTTACGG
CACCTCGACCCCAAAAAACTTGATTAGGGTGATGGTTCACGTAGTGGGCCTCGCCCT
GATAGACGGTTTTTCGCCCTTTGACGTTGGAGTCCACGTTCTTTAATAGTGGACTCTT
GTTCCAAACTGGAACAACACTCAACCCTATCTCGGTCTATTCTTTTGATTTATAAGG
GATTTTGCCGATTTCGGCCTATTGGTTAAAAAATGAGCTGATTTAACAAAAATTTAA
CGCGAATTTTAACAAAATATTAACGTTTACAATTTCCTGATGCGGTATTTTCTCCTTA
CGCATCTGTGCGGTATTTCACACCGCATATGGTGCACTCTCAGTACAATCTGCTCTG
ATGCCGCATAGTTAAGCCAGCCCCGACACCCGCCAACACCCGCTGACGCGCCCTGA
CGGGCTTGTCTGCTCCCGGCATCCGCTTACAGACAAGCTGTGACCGTCTCCGGGAGC
TGCATGTGTCAGAGGTTTTCACCGTCATCACCGAAACGCGCGAGACGAAAGGGCCT
CGTGATACGCCTATTTTTATAGGTTAATGTCATGATAATAATGGTTTCTTAGACGTC
AGGTGGCACTTTTCGGGGAAATGTGCGCGGAACCCCTATTTGTTTATTTTTCTAAAT
ACATTCAAATATGTATCCGCTCATGAGACAATAACCCTGATAAATGCTTCAATAATA
TTGAAAAAGGAAGAGTATGAGTATTCAACATTTCCGTGTCGCCCTTATTCCCTTTTT
TGCGGCATTTTGCCTTCCTGTTTTTGCTCACCCAGAAACGCTGGTGAAAGTAAAAGA
TGCTGAAGATCAGTTGGGTGCAGAGTGGGTTACATCGAACTGGATCTCAACAGCGG
TAAGATCCTTGAGAGTTTTCGCCCCGAAGAACGTTTTCCAATGATGAGCACTTTTAA
AGTTCTGCTATGTGGCGCGGTATTATCCCGTATTGACGCCGGGCAAGAGCAACTCG
GTCGCCGCATACACTATTCTCAGAATGACTTGGTTGAGTACTCACCAGTCACAGAAA
AGCATCTTACGGATGGCATGACAGTAAGAGAATTATGCAGTGCTGCCATAACCATG
AGTGATAACACTGCGGCCAACTTACTTCTGACAACGATCGGAGGACCGAAGGAGCT
AACCGCTTTTTTGCACAACATGGGGGATCATGTAACTCGCCTTGATCGTTGGGAACC
GGAGCTGAATGAAGCCATACCAAACGACGAGCGTGACACCACGATGCCTGTAGCA
ATGGCAACAACGTTGCGCAAACTATTAACTGGCGAACTACTTACTCTAGCTTCCCGG
CAACAATTAATAGACTGGATGGAGGCGGATAAAGTTGCAGGACCACTTCTGCGCTC
GGCCCTTCCGGCTGGCTGGTTTATTGCTGATAAATCTGGAGCCGGTGAGCGTGGGTC
TCGCGGTATCATTGCAGCACTGGGGCCAGATGGTAAGCCCTCCCGTATCGTAGTTAT
CTACACGACGGGGAGTCAGGCAACTATGGATGAACGAAATAGACAGATCGCTGAG
ATAGGTGCCTCACTGATTAAGCATTGGTAACTGTCAGACCAAGTTTACTCATATATA
CTTTAGATTGATTTAAAACTTCATTTTTAATTTAAAAGGATCTAGGTGAAGATCCTTT
TTGATAATCTCATGACCAAAATCCCTTAACGTGAGTTTTCGTTCCACTGAGCGTCAG
ACCCCGTAGAAAAGATCAAAGGATCTTCTTGAGATCCTTTTTTTCTGCGCGTAATCT
GCTGCTTGCAAACAAAAAAACCACCGCTACCAGCGGTGGTTTGTTTGCCGGATCAA
GAGCTACCAACTCTTTTTCCGAAGGTAACTGGCTTCAGCAGAGCGCAGATACCAAA
TACTGTCCTTCTAGTGTAGCCGTAGTTAGGCCACCACTTCAAGAACTCTGTAGCACC
GCCTACATACCTCGCTCTGCTAATCCTGTTACCAGTGGCTGCTGCCAGTGGCGATAA
GTCGTGTCTTACCGGGTTGGACTCAAGACGATAGTTACCGGATAAGGCGCAGCGGT
CGGGCTGAACGGGGGGTTCGTGCACACAGCCCAGCTTGGAGCGAACGACCTACACC
GAACTGAGATACCTACAGCGTGAGCATTGAGAAAGCGCCACGCTTCCCGAAGGGAG
AAAGGCGGACAGGTATCCGGTAAGCGGCAGGGTCGGAACAGGAGAGCGCACGAGG
GAGCTTCCAGGGGGAAACGCCTGGTATCTTTATAGTCCTGTCGGGTTTCGCCACCTC
TGACTTGAGCGTCGATTTTTGTGATGCTCGTCAGGGGGGCGGAGCCTATGGAAAAA
CGCCAGCAACGCGGCCTTTTTACGGTTCCTGGCCTTTTGCTGGCCTTTTGCTCACATG
TTCTTTCCTGCGTTATCCCCTGATTCTGTGGATAACCGTATTACCGCCTTTGAGTGAG
CTGATACCGCTCGCCGCAGCCGAACGACCGAGCGCAGCGAGTCAGTGAGCGAGGA
AGCGGAAGAGCGCCCAATACGCAAACCGCCTCTCCCCGCGCGTTGGCCGATTCATT
AATGCAGCTGGGCTGCAGGGGGGGGGGGGGGGGGGTGGGGGGGGGGGGGGGGGG
>SEQ ID NO: 6: P2i-mHexAB nucleic acid sequence
TTGGCCACTCCCTCTCTGCGCGCTCGCTCGCTCACTGAGGCCGGGCGACCAAAGGTC
GCCCGACGCCCGGGCTTTGCCCGGGCGGCCTCAGTGAGCGAGCGAGCGCGCAGAGA
GGGAGTGGCCAACTCCATCACTAGGGGTTCCTAGATCTGAATTCCTGGAGCCGGTG
TCAGGTTGCTCCGGTAACGGTGACGTGCACGCGTGGGCGGAGCCATCACGCAGGTT
GCTATATAAGCAGAGCTCGTTTAGTGAACCGTCAGAATCGATCCACCATGGCCGGC
TGCAGGCTCTGGGTTTCGCTGCTGCTGGCGGCGGCGTTGGCTTGCTTGGCCACGGCA
CTGTGGCCGTGGCCCCAGTACATCCAAACCTACCACCGGCGCTACACCCTGTACCCC
AACAACTTCCAGTTCCGGTACCATGTCAGTTCGGCCGCGCAGGCGGGCTGCGTCGTC
CTCGACGAGGCCTTTCGACGCTACCGTAACCTGCTCTTCGGTTCCGGCTCTTGGCCC
CGACCCAGCTTCTCAAATAAACAGCAAACGTTGGGGAAGAACATTCTGGTGGTCTC
CGTCGTCACAGCTGAATGTAATGAATTTCCTAATTTGGAGTCGGTAGAAAATTACAC
CCTAACCATTAATGATGACCAGTGTTTACTCGCCTCTGAGACTGTCTGGGGCGCTCT
CCGAGGTCTGGAGACTTTCAGTCAGCTTGTTTGGAAATCAGCTGAGGGCACGTTCTT
TATCAACAAGACAAAGATTAAAGACTTTCCTCGATTCCCTCACCGGGGCGTACTGCT
GGATACATCTCGCCATTACCTGCCATTGTCTAGCATCCTGGATACACTGGATGTCAT
GGCATACAATAAATTCAACGTGTTCCACTGGCACTTGGTGGACGACTCTTCCTTCCC
ATATGAGAGCTTCACTTTCCCAGAGCTCACCAGAAAGGGGTCCTTCAACCCTGTCAC
TCACATCTACACAGCACAGGATGTGAAGGAGGTCATTGAATACGCAAGGCTTCGGG
GTATCCGTGTGCTGGCAGAATTTGACACTCCTGGCCACACTTTGTCCTGGGGGCCAG
GTGCCCCTGGGTTATTAACACCTTGCTACTCTGGGTCTCATCTCTCTGGCACATTTGG
ACCGGTGAACCCCAGTCTCAACAGCACCTATGACTTCATGAGCACACTCTTCCTGGA
GATCAGCTCAGTCTTCCCGGACTTTTATCTCCACCTGGGAGGGGATGAAGTCGACTT
CACCTGCTGGAAGTCCAACCCCAACATCCAGGCCTTCATGAAGAAAAAGGGCTTTA
CTGACTTCAAGCAGCTGGAGTCCTTCTACATCCAGACGCTGCTGGACATCGTCTCTG
ATTATGACAAGGGCTATGTGGTGTGGCAGGAGGTATTTGATAATAAAGTGAAGGTT
CGGCCAGATACAATCATACAGGTGTGGCGGGAAGAAATGCCAGTAGAGTACATGTT
GGAGATGCAAGATATCACCAGGGCTGGCTTCCGGGCCCTGCTGTCTGCTCCCTGGTA
CCTGAACCGTGTAAAGTATGGCCCTGACTGGAAGGACATGTACAAAGTGGAGCCCC
TGGCGTTTCATGGTACGCCTGAACAGAAGGCTCTGGTCATTGGAGGGGAGGCCTGT
ATGTGGGGAGAGTATGTGGACAGCACCAACCTGGTCCCCAGACTCTGGCCCAGAGC
GGGTGCCGTCGCTGAGAGACTGTGGAGCAGTAACCTGACAACTAATATAGACTTTG
CCTTTAAACGTTTGTCGCATTTCCGTTGTGAGCTGGTGAGGAGAGGAATCCAGGCCC
AGCCCATCAGTGTAGGCTACTGTGAGCAGGAGTTTGAGCAGACTTGAGCGGCCGCG
ACTAGAGCTCGCTGATCAGCCTCGACTGTGCCTTCTAGTTGCCAGCCATCTGTTGTT
TGCCCCTCCCCCGTGCCTTCCTTGACCCTGGAAGGTGCCACTCCCACTGTCCTTTCCT
AATAAAATGAGGAAATTGCATCGCATTGTCTGAGTAGGTGTCATTCTATTCTGGGGG
GTGGGGTGGGGCAGGACAGCAAGGGGGAGGATTGGGAAGACAATAGCAGTACCAC
ATTTGTAGAGGTTTTACTTGCTTTAAAAAACCTCCCACATCTCCCCCTGAACCTGAA
ACATAAAATGAATGCAATTGTTGTTGTTAACTTGTTTATTGCAGCTTATAATGGTTA
CAAATAAAGCAATAGCATCACAAATTTCACAAATAAAGCATTTTTTTCACTGCATTC
TAGTTGTGGTTTGTCCAAACTCATCAATGTATCTTATCATGTCTGCTCGACCTCGAGC
TATATTTTATTCTCATAGTTACAGTATCCAGTATAGAGAGGTTGTGCAGCTATTCCA
CGGCTGACCATTCTGCAGCGGTGCACGGCCAGTCGTTTGTAGGCATTTTCTAGGTCA
GTGACAGTTTTAGGGCTCCAGAGTCTCTCACCAACAGCGCTTGCTCGAGGCCATAAT
CTTGGAGTAAGGTTAGTTGCATCCACAAATTCTCCCCACAGGCAAGCTTCTCCACCA
ATAACAAGTTGTTTCTGCTTCTCAGAGCCTTCAAAATTAAGGGGCTCAACTTTGTAG
TAGTTTTTCCAGTCTTGCCCATAGCTGATCAGGTCTAAGTACCAAGGAGCAGAAAG
GATGGCAGGGAAGCCAGAGCCTGTGACTTGCTTTAGCTCATATGAATAATGCTCAC
TCTTCCACACTTCGACTACTGTGCCCGGCTGAAGCTCCACCTTATCATCAAAAACTT
CTTGCCAAACAATGGAGTTCTTCTTTAAGGATGAAATAATTTCCAAAATCTTTTTAA
TATAAAAGGATTCTAGTCTTCTAAAATCGCTGCCAAAGCCCTTTCTCTTCATGAAAC
CTTGGATGTTTGGATTTGATGCCCAACATTGAAATTCTACTTCATCTCCTCCCAAGTG
GATGAACTGATCTGGAAACACACTGCTGATTTCTTTGAAAAATGTGTTAAAGAATG
CATACGTTGTGTTTACAGTTGGGTCTACAGGCCCAAACACTTGAGTTTTAGTTTTTTG
ATTGTAACATGGAGTTAGAAGGTTTTTCTGTCCTTTGCCCCAAGACTGTGTATGGCC
AGGGGTATCAAATTCTGGTATGACTCGAATCCCTCGGAGCCGGGCGTACTCCAGCA
CCATCCGGACATCGTTTGGTGTATAGACATGAGACAAAGAGTAGCTTCCCTTATTGC
TTAGCTCAGGAAAAGTGGTACTCTGATAAGGGAAAGACTGGTCGTCCACTATGTGC
CAGTGAAGAACATTAAACTTATTAAAAGCCATGGCATCCAGAGTTTTTAAAATTGTC
TTCACAGGCAGGAAGTGTCTAGATGTATCAATTAAAATTCCTCTATGAGGGAATCTT
GGAGAATCAGCTATACTGGATTCATTGATGGTGAAAGTCCCGAAAGAGTCTTGGTA
AACTAACTGGCTAAACGTCTCTAAACCTCGTAACGCTCCCCAAACGCTGTTGGCCTT
GAGGACGGCTACTGGTTCTTGTACAAGCAGAGAATAGGTTTCATCTGAAGACAGAC
TAGGGAAGGACTCGCACTCTGACTCGAGGGTAATGGAGACCAGGAGCTTCTGCAAC
TGTGGCTCAGCTCGAAATCTAGCAGGGCCATGATGTCTCTTGTAGAAACCAAAAAC
ATAGTTGTAATATCGCCGAAACGCCTCCTGTAGCAGCGAGCAGGAAGGGCCCGCTG
TGGAATTGGGACTGTGGTCGATGCTGAAGTCCTCCGCGGAGATGTACAACAGCCGC
GGGAACATCTGCACCGAGCGCGGGAAGGGCCATAGCGCAGGTTGCAGTCGGGCCG
GGGCCACTAGGGCCAGCGACACTAGCGACACCAGCGCCTGCAGCAGCAGCAGCCC
GGGGGCGCTACGCGGGGACTGCGGCATGGTGGACTAGTTCTGACGGTTCACTAAAC
GAGCTCTGCTTATATAGCAACCTGCGTGATGGCTCCGCCCACGCGTGCACGTCACCG
TTACCGGAGCAACCTGACACCGGCTCCAGGCATGCTGGGGAGAGATCTAGGAACCC
CTAGTGATGGAGTTGGCCACTCCCTCTCTGCGCGCTCGCTCGCTCACTGAGGCCGCC
CGGGCAAAGCCCGGGCGTCGGGCGACCTTTGGTCGCCCGGCCTCAGTGAGCGAGCG
AGCGCGCAGAGAGGGAGTGGCCATGCAGCCAGCTGGCGTAATAGCGAAGAGGCCC
GCACCGATCGCCCTTCCCAACAGTTGCGTAGCCTGAATGGCGAATGGCGCGACGCG
CCCTGTAGCGGCGCATTAAGCGCGGCGGGTGTGGTGGTTACGCGCAGCGTGACCGC
TACACTTGCCAGCGCCCTAGCGCCCGCTCCTTTCGCTTTCTTCCCTTCCTTTCTCGCC
ACGTTCGCCGGCTTTCCCCGTCAAGCTCTAAATCGGGGGCTCCCTTTAGGGTTCCGA
TTTAGTGCTTTACGGCACCTCGACCCCAAAAAACTTGATTAGGGTGATGGTTCACGT
AGTGGGCCATCGCCCTGATAGACGGTTTTTCGCCCTTTGACGTTGGAGTCCACGTTC
TTTAATAGTGGACTCTTGTTCCAAACTGGAACAACACTCAACCCTATCTCGGTCTAT
TCTTTTGATTTATAAGGGATTTTGCCGATTTCGGCCTATTGGTTAAAAAATGAGCTG
ATTTAACAAAAATTTAACGCGAATTTTAACAAAATATTAACGTTTACAATTTCCTGA
TGCGGTATTTTCTCCTTACGCATCTGTGCGGTATTTCACACCGCATATGGTGCACTCT
CAGTACAATCTGCTCTGATGCCGCATAGTTAAGCCAGCCCCGACACCCGCCAACAC
CCGCTGACGCGCCCTGACGGGCTTGTCTGCTCCCGGCATCCGCTTACAGACAAGCTG
TGACCGTCTCCGGGAGCTGCATGTGTCAGAGGTTTTCACCGTCATCACCGAAACGCG
CGAGACGAAAGGGCCTCGTGATACGCCTATTTTTATAGGTTAATGTCATGATAATAA
TGGTTTCTTAGACGTCAGGTGGCACTTTTCGGGGAAATGTGCGCGGAACCCCTATTT
GTTTATTTTTCTAAATACATTCAAATATGTATCCGCTCATGAGACAATAACCCTGAT
AAATGCTTCAATAATATTGAAAAAGGAAGAGTATGAGTATTCAACATTTCCGTGTC
GCCCTTATTCCCTTTTTTGCGGCATTTTGCCTTCCTGTTTTTGCTCACCCAGAAACGC
TGGTGAAAGTAAAAGATGCTGAAGATCAGTTGGGTGCACGAGTGGGTTACATCGAA
CTGGATCTCAACAGCGGTAAGATCCTTGAGAGTTTTCGCCCCGAAGAACGTTTTCCA
ATGATGAGCACTTTTAAAGTTCTGCTATGTGGCGCGGTATTATCCCGTATTGACGCC
GGGCAAGAGCAACTCGGTCGCCGCATACACTATTCTCAGAATGACTTGGTTGAGTA
CTCACCAGTCACAGAAAAGCATCTTACGGATGGCATGACAGTAAGAGAATTATGCA
GTGCTGCCATAACCATGAGTGATAACACTGCGGCCAACTTACTTCTGACAACGATC
GGAGGACCGAAGGAGCTAACCGCTTTTTTGCACAACATGGGGGATCATGTAACTCG
CCTTGATCGTTGGGAACCGGAGCTGAATGAAGCCATACCAAACGACGAGCGTGACA
CCACGATGCCTGTAGCAATGGCAACAACGTTGCGCAAACTATTAACTGGCGAACTA
CTTACTCTAGCTTCCCGGCAACAATTAATAGACTGGATGGAGGCGGATAAAGTTGC
AGGACCACTTCTGCGCTCGGCCCTTCCGGCTGGCTGGTTTATTGCTGATAAATCTGG
AGCCGGTGAGCGTGGGTCTCGCGGTATCATTGCAGCACTGGGGCCAGATGGTAAGC
CCTCCCGTATCGTAGTTATCTACACGACGGGGAGTCAGGCAACTATGGATGAACGA
AATAGACAGATCGCTGAGATAGGTGCCTCACTGATTAAGCATTGGTAACTGTCAGA
CCAAGTTTACTCATATATACTTTAGATTGATTTAAAACTTCATTTTTAATTTAAAAGG
ATCTAGGTGAAGATCCTTTTTGATAATCTCATGACCAAAATCCCTTAACGTGAGTTT
TCGTTCCACTGAGCGTCAGACCCCGTAGAAAAGATCAAAGGATCTTCTTGAGATCCT
TTTTTTCTGCGCGTAATCTGCTGCTTGCAAACAAAAAAACCACCGCTACCAGCGGTG
GTTTGTTTGCCGGATCAAGAGCTACCAACTCTTTTTCCGAAGGTAACTGGCTTCAGC
AGAGCGCAGATACCAAATACTGTCCTTCTAGTGTAGCCGTAGTTAGGCCACCACTTC
AAGAACTCTGTAGCACCGCCTACATACCTCGCTCTGCTAATCCTGTTACCAGTGGCT
GCTGCCAGTGGCGATAAGTCGTGTCTTACCGGGTTGGACTCAAGACGATAGTTACC
GGATAAGGCGCAGCGGTCGGGCTGAACGGGGGGTTCGTGCACACAGCCCAGCTTGG
AGCGAACGACCTACACCGAACTGAGATACCTACAGCGTGAGCATTGAGAAAGCGCC
ACGCTTCCCGAAGGGAGAAAGGCGGACAGGTATCCGGTAAGCGGCAGGGTCGGAA
CAGGAGAGCGCACGAGGGAGCTTCCAGGGGGAAACGCCTGGTATCTTTATAGTCCT
GTCGGGTTTCGCCACCTCTGACTTGAGCGTCGATTTTTGTGATGCTCGTCAGGGGGG
CGGAGCCTATGGAAAAACGCCAGCAACGCGGCCTTTTTACGGTTCCTGGCCTTTTGC
TGGCCTTTTGCTCACATGTTCTTTCCTGCGTTATCCCCTGATTCTGTGGATAACCGTA
TTACCGCCTTTGAGTGAGCTGATACCGCTCGCCGCAGCCGAACGACCGAGCGCAGC
GAGTCAGTGAGCGAGGAAGCGGAAGAGCGCCCAATACGCAAACCGCCTCTCCCCG
CGCGTTGGCCGATTCATTAATGCAGCTGGGCTGCAGGGGGGGGGGGGGGGGGGTG
GGGGGGGGGGGGGGGGG
>SEQ ID NO: 7: AAV-hHexA:codon optimized-BiCBA-hHexB nucleic
acid sequence
TTGGCCACTCCCTCTCTGCGCGCTCGCTCGCTCACTGAGGCCGGGCGACCAAAGGTC
GCCCGACGCCCGGGCTTTGCCCGGGCGGCCTCAGTGAGCGAGCGAGCGCGCAGAGA
GGGAGTGGCCAACTCCATCACTAGGGGTTCCTAGATCTGAATTCTACCACATTTGTA
GAGGTTTTACTTGCTTTAAAAAACCTCCCACATCTCCCCCTGAACCTGAAACATAAA
ATGAATGCAATTGTTGTTGTTAACTTGTTTATTGCAGCTTATAATGGTTACAAATAA
AGCAATAGCATCACAAATTTCACAAATAAAGCATTTTTTTCACTGCATTCTAGTTGT
GGTTTGTCCAAACTCATCAATGTATCTTATCATGTCTGTCGAGGCGGCCGCTCAGGT
CTGCTCAAATTCCTGCTCACAGAATCCGACATTCAGGGGCTGTGCCTGCACGCCTCT
CCTCAGCAGCTCGCACCTGAAGTGGCTCAGCCTTTCGTAAGCGAATGTCAGATCAG
AGGTCAGCTTGTTGGACCACAGTCTCTCGGCCACTGCTCCGGCCCTGGGCCACAGTC
GTGGGACCAGATTGGTGTTGTCCACATACTCGCCCCACATACATGCTTCGCCCCCGA
TGACCAGAGCTTTCTGCTCAGGAGTCCCTTCAAAGGCCAGGGGCTCCACAATGTAG
AAATCCTTCCAGTCTGGTCCATATGAGATCCTGTTCAGGTACCAAGGGGCACTCAGC
AGTGCTCGGAAGCCAGCTTTTGTCACCAGTTCCAGCTCCTTCATATAATTGACAGGA
ATATCCTCGCGCCACACTTGAATGATGGTATCGGGCTGGATTTTGACCTTGTTGTCA
AACACTTCCTGCCAGACCACATAGCCCTTTCCGTATGAACTCACAATGTCCAGCAGA
GTCTGGATGTAGAAACTCTCCAGCTGCTTGAAGTCTTCGCCGAACCCTTTCTTCCGC
ATAAAGTCCTGGATCTCTGGATTAGACTTCCAACATGTGAAATCCACTTCGTCTCCG
CCCAGGTGCAGATAAAAGTCAGGGAAGACAGAGGACACCTCCAGAAAGAATGTAG
ACATGAACTCGTAGGTATTGTTCAGGGATGGGTTCACAGGTCCGAAGGTGCCGCTT
GGCTCACTCCCAGAGTAGCAAGGAGTCAGCAGTCCGGGAATTCCTGGGCCCCAGCT
CAGAGTATGGCCAGGTGTATCGAATTCAGCCAGCACTCGAATCCCCCTCAGTCGGG
CATACTCGATGACTTCTTTCACGTCCTGGGCGGTATAGATGTGAGTCACGGGATTGT
AGCTGCCCTTGCGCATCAGCTCTGGAAATGTGAAGCTTTCGTAAGGAAATGAGGGA
TCGTCGACCAGATGCCAGTGGAACACGTTCAGCTTGTTATAAGCCATCACATCCAG
AGTGTCCAGAATGCTTGACAGGGGCAGGTAATGTCTAGATGTATCCAGCAGCAGCC
CCCGGTGTGGAAATCTAGGGAAGTCTTCGATCTCAGTTTTGTTGATAAAGAATGTTC
CCTCGGCTGACTTCCAGACCAGCTGACTGAAAGTCTCCAGGCCCCTCAGTGCCCCCC
ACACTGTTTCGGACAGCAGCAGACACTGATCGTCATTGATTGTCAGGGTATAGTTCT
CCACGGATTCCAGGGTGGGCAGCTGATTGCAGCCTGGAGTGACCACGCTGACCACC
AGCACGTTTTTCTCCAGGGTGTGGCGCTTCCCAGTCAGATAGGGCCGTGGCCAGGA
CCCGCTTCCAAACAGCAGATCGCGGTACCGCTGGAATGCCTCGTCCAGGACGCTGC
ATCCTGGCTGAGCGGCGCTGCTCACGTCGTACTGAAACTGGAAATTGTTTGGATACA
GCACGTACCTCTGGTCAGATGTCTGGAAGTTCTGAGGCCATGGCCACAGGGCGGTT
GCTCGTCCAGCAAATGCAGCGGCCAGCAGCAGGGAGAACCACAGTCTGGAGCTGGT
CATGGTGGCTTTATCGATACTAGCGCTAGAGCTTGCTCCCGCCCGCCGCGCGCTTCG
CTTTTTATAGGGCCGCCGCCGCCGCCGCCTCGCCATAAAAGGAAACTTTCGGAGCG
CGCCGCTCTGATTGGCTGCCGCCGCACCTCTCCGCCTCGCCCCGCCCCGCCCCTCGC
CCCCATCGCTGCACAAAATAATTAAAAAATAAATAAATACAAAATTGGGGGTGGGG
AGGGGGGGGAGATGGGGAGAGTGAAGCAGAACGTGGCCTCGGATCCCCCGGGCTG
CAGTATTAATAGTAATCAATTACGGGGTCATTAGTTCATAGCCCATATATGGAGTTC
CGCGTTACATAACTTACGGTAAATGGCCCGCCTGGCTGACCGCCCAACGACCCCCG
CCCATTGACGTCAATAATGACGTATGTTCCCATAGTAACGCCAATAGGGACTTTCCA
TTGACGTCAATGGGTGGAGTATTTACGGTAAACTGCCCACTTGGCAGTACATCAAGT
GTATCATATGCCAAGTACGCCCCCTATTGACGTCAATGACGGTAAATGGCCCGCCTG
GCATTATGCCCAGTACATGACCTTATGGGACTTTCCTACTTGGCAGTACATCTACGT
ATTAGTCATCGCTATTACCATGTCGAGGCCACGTTCTGCTTCACTCTCCCCATCTCCC
CCCCCTCCCCACCCCCAATTTTGTATTTATTTATTTTTTAATTATTTTGTGCAGCGAT
GGGGGCGAGGGGCGGGGCGGGGCGAGGCGGAGAGGTGCGGCGGCAGCCAATCAG
AGCGGCGCGCTCCGAAAGTTTCCTTTTATGGCGAGGCGGCGGCGGCGGCGGCCCTA
TAAAAAGCGAAGCGCGCGGCGGGCGGGAGCAAGCTCGCTAGCACTAGTGGAGCAC
CATGGAGCTGTGCGGGCTGGGGCTGCCCCGGCCGCCCATGCTGCTGGCGCTGCTGTT
GGCGACACTGCTGGCGGCGATGTTGGCGCTGCTGACTCAGGTGGCGCTGGTGGTGC
AGGTGGCGGAGGCGGCTCGGGCCCCGAGCGTCTCGGCCAAGCCGGGGCCGGCGCT
GTGGCCCCTGCCGCTCTCGGTGAAGATGACCCCGAACCTGCTGCATCTCGCCCCGGA
GAACTTCTACATCAGCCACAGCCCCAATTCCACGGCGGGCCCCTCCTGCACCCTGCT
GGAGGAAGCGTTTCGACGATATCATGGCTATATTTTTGGTTTCTACAAGTGGCATCA
TGAACCTGCTGAATTCCAGGCTAAAACCCAGGTTCAGCAACTTCTTGTCTCAATCAC
CCTTCAGTCAGAGTGTGATGCTTTCCCCAACATATCTTCAGATGAGTCTTATACTTTA
CTTGTGAAAGAACCAGTGGCTGTCCTTAAGGCCAACAGAGTTTGGGGAGCATTACG
AGGTTTAGAGACCTTTAGCCAGTTAGTTTATCAAGATTCTTATGGAACTTTCACCAT
CAATGAATCCACCATTATTGATTCTCCAAGGTTTTCTCACAGAGGAATTTTGATTGA
TACATCCAGACATTATCTGCCAGTTAAGATTATTCTTAAAACTCTGGATGCCATGGC
TTTTAATAAGTTTAATGTTCTTCACTGGCACATAGTTGATGACCAGTCTTTCCCATAT
CAGAGCATCACTTTTCCTGAGTTAAGCAATAAAGGAAGCTATTCTTTGTCTCATGTT
TATACACCAAATGATGTCCGTATGGTGATTGAATATGCCAGATTACGAGGAATTCG
AGTCCTGCCAGAATTTGATACCCCTGGGCATACACTATCTTGGGGAAAAGGTCAGA
AAGACCTCCTGACTCCATGTTACAGTAGACAAAACAAGTTGGACTCTTTTGGACCTA
TAAACCCTACTCTGAATACAACATACAGCTTCCTTACTACATTTTTCAAAGAAATTA
GTGAGGTGTTTCCAGATCAATTCATTCATTTGGGAGGAGATGAAGTGGAATTTAAAT
GTTGGGAATCAAATCCAAAAATTCAAGATTTCATGAGGCAAAAAGGCTTTGGCACA
GATTTTAAGAAACTAGAATCTTTCTACATTCAAAAGGTTTTGGATATTATTGCAACC
ATAAACAAGGGATCCATTGTCTGGCAGGAGGTTTTTGATGATAAAGCAAAGCTTGC
GCCGGGCACAATAGTTGAAGTATGGAAAGACAGCGCATATCCTGAGGAACTCAGTA
GAGTCACAGCATCTGGCTTCCCTGTAATCCTTTCTGCTCCTTGGTACTTAGATTTGAT
TAGCTATGGACAAGATTGGAGGAAATACTATAAAGTGGAACCTCTTGATTTTGGCG
GTACTCAGAAACAGAAACAACTTTTCATTGGTGGAGAAGCTTGTCTATGGGGAGAA
TATGTGGATGCAACTAACCTCACTCCAAGATTATGGCCTCGGGCAAGTGCTGTTGGT
GAGAGACTCTGGAGTTCCAAAGATGTCAGAGATATGGATGACGCCTATGACAGACT
GACAAGGCACCGCTGCAGGATGGTCGAACGTGGAATAGCTGCACAACCTCTTTATG
CTGGATATTGTAACCATGAGAACATGTAACTCGAGGCGGCCGCTCTAGAGATCTTTT
TCCCTCTGCCAAAAATTATGGGGACATCATGAAGCCCCTTGAGCATCTGACTTCTGG
CTAATAAAGGAAATTTATTTTCATTGCAATAGTGTGTTGGAATTTTTTGTGTCTCTCA
CTCGGCATGCTGGGGAGAGATCTGAGGAACCCCTAGTGATGGAGTTGGCCACTCCC
TCTCTGCGCGCTCGCTCGCTCACTGAGGCCGCCCGGGCAAAGCCCGGGCGTCGGGC
GACCTTTGGTCGCCCGGCCTCAGTGAGCGAGCGAGCGCGCAGAGAGGGAGTGGCCA
TGCAGCCAGCTGGCGTAATAGCGAAGAGGCCCGCACCGATCGCCCTTCCCAACAGT
TGCGTAGCCTGAATGGCGAATGGCGCGACGCGCCCTGTAGCGGCGCATTAAGCGCG
GCGGGTGTGGTGGTTACGCGCAGCGTGACCGCTACACTTGCCAGCGCCCTAGCGCC
CGCTCCTTTCGCTTTCTTCCCTTCCTTTCTCGCCACGTTCGCCGGCTTTCCCCGTCAA
GCTCTAAATCGGGGGCTCCCTTTAGGGTTCCGATTTAGTGCTTTACGGCACCTCGAC
CCCAAAAAACTTGATTAGGGTGATGGTTCACGTAGTGGGCCATCGCCCTGATAGAC
GGTTTTTCGCCCTTTGACGTTGGAGTCCACGTTCTTTAATAGTGGACTCTTGTTCCAA
ACTGGAACAACACTCAACCCTATCTCGGTCTATTCTTTTGATTTATAAGGGATTTTG
CCGATTTCGGCCTATTGGTTAAAAAATGAGCTGATTTAACAAAAATTTAACGCGAAT
TTTAACAAAATATTAACGTTTACAATTTCCTGATGCGGTATTTTCTCCTTACGCATCT
GTGCGGTATTTCACACCGCATATGGTGCACTCTCAGTACAATCTGCTCTGATGCCGC
ATAGTTAAGCCAGCCCCGACACCCGCCAACACCCGCTGACGCGCCCTGACGGGCTT
GTCTGCTCCCGGCATCCGCTTACAGACAAGCTGTGACCGTCTCCGGGAGCTGCATGT
GTCAGAGGTTTTCACCGTCATCACCGAAACGCGCGAGACGAAAGGGCCTCGTGATA
CGCCTATTTTTATAGGTTAATGTCATGATAATAATGGTTTCTTAGACGTCAGGTGGC
ACTTTTCGGGGAAATGTGCGCGGAACCCCTATTTGTTTATTTTTCTAAATACATTCA
AATATGTATCCGCTCATGAGACAATAACCCTGATAAATGCTTCAATAATATTGAAA
AAGGAAGAGTATGAGTATTCAACATTTCCGTGTCGCCCTTATTCCCTTTTTTGCGGC
ATTTTGCCTTCCTGTTTTTGCTCACCCAGAAACGCTGGTGAAAGTAAAAGATGCTGA
AGATCAGTTGGGTGCACGAGTGGGTTACATCGAACTGGATCTCAACAGCGGTAAGA
TCCTTGAGAGTTTTCGCCCCGAAGAACGTTTTCCAATGATGAGCACTTTTAAAGTTC
TGCTATGTGGCGCGGTATTATCCCGTATTGACGCCGGGCAAGAGCAACTCGGTCGC
CGCATACACTATTCTCAGAATGACTTGGTTGAGTACTCACCAGTCACAGAAAAGCA
TCTTACGGATGGCATGACAGTAAGAGAATTATGCAGTGCTGCCATAACCATGAGTG
ATAACACTGCGGCCAACTTACTTCTGACAACGATCGGAGGACCGAAGGAGCTAACC
GCTTTTTTGCACAACATGGGGGATCATGTAACTCGCCTTGATCGTTGGGAACCGGAG
CTGAATGAAGCCATACCAAACGACGAGCGTGACACCACGATGCCTGTAGCAATGGC
AACAACGTTGCGCAAACTATTAACTGGCGAACTACTTACTCTAGCTTCCCGGCAACA
ATTAATAGACTGGATGGAGGCGGATAAAGTTGCAGGACCACTTCTGCGCTCGGCCC
TTCCGGCTGGCTGGTTTATTGCTGATAAATCTGGAGCCGGTGAGCGTGGGTCTCGCG
GTATCATTGCAGCACTGGGGCCAGATGGTAAGCCCTCCCGTATCGTAGTTATCTACA
CGACGGGGAGTCAGGCAACTATGGATGAACGAAATAGACAGATCGCTGAGATAGG
TGCCTCACTGATTAAGCATTGGTAACTGTCAGACCAAGTTTACTCATATATACTTTA
GATTGATTTAAAACTTCATTTTTAATTTAAAAGGATCTAGGTGAAGATCCTTTTTGA
TAATCTCATGACCAAAATCCCTTAACGTGAGTTTTCGTTCCACTGAGCGTCAGACCC
CGTAGAAAAGATCAAAGGATCTTCTTGAGATCCTTTTTTTCTGCGCGTAATCTGCTG
CTTGCAAACAAAAAAACCACCGCTACCAGCGGTGGTTTGTTTGCCGGATCAAGAGC
TACCAACTCTTTTTCCGAAGGTAACTGGCTTCAGCAGAGCGCAGATACCAAATACTG
TCCTTCTAGTGTAGCCGTAGTTAGGCCACCACTTCAAGAACTCTGTAGCACCGCCTA
CATACCTCGCTCTGCTAATCCTGTTACCAGTGGCTGCTGCCAGTGGCGATAAGTCGT
GTCTTACCGGGTTGGACTCAAGACGATAGTTACCGGATAAGGCGCAGCGGTCGGGC
TGAACGGGGGGTTCGTGCACACAGCCCAGCTTGGAGCGAACGACCTACACCGAACT
GAGATACCTACAGCGTGAGCATTGAGAAAGCGCCACGCTTCCCGAAGGGAGAAAG
GCGGACAGGTATCCGGTAAGCGGCAGGGTCGGAACAGGAGAGCGCACGAGGGAGC
TTCCAGGGGGAAACGCCTGGTATCTTTATAGTCCTGTCGGGTTTCGCCACCTCTGAC
TTGAGCGTCGATTTTTGTGATGCTCGTCAGGGGGGCGGAGCCTATGGAAAAACGCC
AGCAACGCGGCCTTTTTACGGTTCCTGGCCTTTTGCTGGCCTTTTGCTCACATGTTCT
TTCCTGCGTTATCCCCTGATTCTGTGGATAACCGTATTACCGCCTTTGAGTGAGCTG
ATACCGCTCGCCGCAGCCGAACGACCGAGCGCAGCGAGTCAGTGAGCGAGGAAGC
GGAAGAGCGCCCAATACGCAAACCGCCTCTCCCCGCGCGTTGGCCGATTCATTAAT
GCAGCTGGGCTGCAGGGGGGGGGGGGGGGGGGTGGGGGGGGGGGGGGGGGG
>SEQ ID NO: 8: AAV-hHexA:codon optimized-BiCBA-hHexB: codon
optimized nucleic acid sequence
TTGGCCACTCCCTCTCTGCGCGCTCGCTCGCTCACTGAGGCCGGGCGACCAAAGGTC
GCCCGACGCCCGGGCTTTGCCCGGGCGGCCTCAGTGAGCGAGCGAGCGCGCAGAGA
GGGAGTGGCCAACTCCATCACTAGGGGTTCCTCAGATCTGAATTCTACCACATTTGT
AGAGGTTTTACTTGCTTTAAAAAACCTCCCACATCTCCCCCTGAACCTGAAACATAA
AATGAATGCAATTGTTGTTGTTAACTTGTTTATTGCAGCTTATAATGGTTACAAATA
AAGCAATAGCATCACAAATTTCACAAATAAAGCATTTTTTTCACTGCATTCTAGTTG
TGGTTTGTCCAAACTCATCAATGTATCTTATCATGTCTGTCGAGGCGGCCGCTCAGG
TCTGCTCAAATTCCTGCTCACAGAATCCGACATTCAGGGGCTGTGCCTGCACGCCTC
TCCTCAGCAGCTCGCACCTGAAGTGGCTCAGCCTTTCGTAAGCGAATGTCAGATCAG
AGGTCAGCTTGTTGGACCACAGTCTCTCGGCCACTGCTCCGGCCCTGGGCCACAGTC
GTGGGACCAGATTGGTGTTGTCCACATACTCGCCCCACATACATGCTTCGCCCCCGA
TGACCAGAGCTTTCTGCTCAGGAGTCCCTTCAAAGGCCAGGGGCTCCACAATGTAG
AAATCCTTCCAGTCTGGTCCATATGAGATCCTGTTCAGGTACCAAGGGGCACTCAGC
AGTGCTCGGAAGCCAGCTTTTGTCACCAGTTCCAGCTCCTTCATATAATTGACAGGA
ATATCCTCGCGCCACACTTGAATGATGGTATCGGGCTGGATTTTGACCTTGTTGTCA
AACACTTCCTGCCAGACCACATAGCCCTTTCCGTATGAACTCACAATGTCCAGCAGA
GTCTGGATGTAGAAACTCTCCAGCTGCTTGAAGTCTTCGCCGAACCCTTTCTTCCGC
ATAAAGTCCTGGATCTCTGGATTAGACTTCCAACATGTGAAATCCACTTCGTCTCCG
CCCAGGTGCAGATAAAAGTCAGGGAAGACAGAGGACACCTCCAGAAAGAATGTAG
ACATGAACTCGTAGGTATTGTTCAGGGATGGGTTCACAGGTCCGAAGGTGCCGCTT
GGCTCACTCCCAGAGTAGCAAGGAGTCAGCAGTCCGGGAATTCCTGGGCCCCAGCT
CAGAGTATGGCCAGGTGTATCGAATTCAGCCAGCACTCGAATCCCCCTCAGTCGGG
CATACTCGATGACTTCTTTCACGTCCTGGGCGGTATAGATGTGAGTCACGGGATTGT
AGCTGCCCTTGCGCATCAGCTCTGGAAATGTGAAGCTTTCGTAAGGAAATGAGGGA
TCGTCGACCAGATGCCAGTGGAACACGTTCAGCTTGTTATAAGCCATCACATCCAG
AGTGTCCAGAATGCTTGACAGGGGCAGGTAATGTCTAGATGTATCCAGCAGCAGCC
CCCGGTGTGGAAATCTAGGGAAGTCTTCGATCTCAGTTTTGTTGATAAAGAATGTTC
CCTCGGCTGACTTCCAGACCAGCTGACTGAAAGTCTCCAGGCCCCTCAGTGCCCCCC
ACACTGTTTCGGACAGCAGCAGACACTGATCGTCATTGATTGTCAGGGTATAGTTCT
CCACGGATTCCAGGGTGGGCAGCTGATTGCAGCCTGGAGTGACCACGCTGACCACC
AGCACGTTTTTCTCCAGGGTGTGGCGCTTCCCAGTCAGATAGGGCCGTGGCCAGGA
CCCGCTTCCAAACAGCAGATCGCGGTACCGCTGGAATGCCTCGTCCAGGACGCTGC
ATCCTGGCTGAGCGGCGCTGCTCACGTCGTACTGAAACTGGAAATTGTTTGGATACA
GCACGTACCTCTGGTCAGATGTCTGGAAGTTCTGAGGCCATGGCCACAGGGCGGTT
GCTCGTCCAGCAAATGCAGCGGCCAGCAGCAGGGAGAACCACAGTCTGGAGCTGGT
CATGGTGGCTTTATCGATACTAGCGCTAGAGCTTGCTCCCGCCCGCCGCGCGCTTCG
CTTTTTATAGGGCCGCCGCCGCCGCCGCCTCGCCATAAAAGGAAACTTTCGGAGCG
CGCCGCTCTGATTGGCTGCCGCCGCACCTCTCCGCCTCGCCCCGCCCCGCCCCTCGC
CCCCATCGCTGCACAAAATAATTAAAAAATAAATAAATACAAAATTGGGGGTGGGG
AGGGGGGGGAGATGGGGAGAGTGAAGCAGAACGTGGCCTCGGATCCCCCGGGCTG
CAGTATTAATAGTAATCAATTACGGGGTCATTAGTTCATAGCCCATATATGGAGTTC
CGCGTTACATAACTTACGGTAAATGGCCCGCCTGGCTGACCGCCCAACGACCCCCG
CCCATTGACGTCAATAATGACGTATGTTCCCATAGTAACGCCAATAGGGACTTTCCA
TTGACGTCAATGGGTGGAGTATTTACGGTAAACTGCCCACTTGGCAGTACATCAAGT
GTATCATATGCCAAGTACGCCCCCTATTGACGTCAATGACGGTAAATGGCCCGCCTG
GCATTATGCCCAGTACATGACCTTATGGGACTTTCCTACTTGGCAGTACATCTACGT
ATTAGTCATCGCTATTACCATGTCGAGGCCACGTTCTGCTTCACTCTCCCCATCTCCC
CCCCCTCCCCACCCCCAATTTTGTATTTATTTATTTTTTAATTATTTTGTGCAGCGAT
GGGGGCGAGGGGCGGGGCGGGGCGAGGCGGAGAGGTGCGGCGGCAGCCAATCAG
AGCGGCGCGCTCCGAAAGTTTCCTTTTATGGCGAGGCGGCGGCGGCGGCGGCCCTA
TAAAAAGCGAAGCGCGCGGCGGGCGGGAGCAAGCTCGCTAGCACTAGTAAAGCCA
CCATGGAACTGTGCGGACTGGGACTGCCTAGACCCCCTATGCTGCTGGCTCTGCTGC
TGGCTACCCTGCTGGCTGCTATGCTGGCCCTGCTGACTCAGGTGGCCCTGGTGGTCC
AGGTCGCAGAGGCAGCTAGGGCTCCTTCAGTGAGCGCAAAGCCAGGACCTGCCCTG
TGGCCACTGCCACTGTCCGTGAAAATGACACCAAACCTGCTGCACCTGGCCCCCGA
AAATTTCTACATCTCCCATTCTCCTAACAGTACAGCAGGGCCATCATGCACTCTGCT
GGAGGAAGCCTTCCGGAGATACCACGGGTATATTTTCGGCTTCTACAAGTGGCACC
ATGAGCCCGCAGAATTTCAGGCCAAAACCCAGGTGCAGCAGCTGCTGGTCAGCATC
ACACTGCAGTCCGAGTGTGACGCTTTCCCAAATATTAGCTCCGATGAGAGCTACACC
CTGCTGGTGAAGGAACCCGTGGCTGTCCTGAAAGCAAACAGAGTCTGGGGCGCACT
GAGGGGGCTGGAGACATTCTCCCAGCTGGTGTACCAGGACTCTTACGGAACATTTA
CTATCAACGAATCCACTATCATCGACTCCCCTAGGTTTTCTCACCGCGGCATCCTGA
TTGATACTTCTCGCCATTACCTGCCAGTGAAGATCATTCTGAAAACCCTGGATGCAA
TGGCCTTCAACAAGTTTAATGTGCTGCACTGGCATATCGTCGACGATCAGTCTTTCC
CTTACCAGAGTATTACATTTCCAGAGCTGAGTAATAAAGGCAGTTACTCACTGAGCC
ACGTGTATACTCCAAACGACGTGAGAATGGTCATCGAGTATGCCAGGCTGCGCGGA
ATTAGGGTGCTGCCAGAATTCGATACTCCCGGCCATACCCTGTCTTGGGGCAAGGG
GCAGAAAGACCTGCTGACCCCCTGCTACAGTCGGCAGAACAAGCTGGATTCATTCG
GCCCTATCAACCCAACACTGAATACCACATACTCTTTTCTGACTACCTTCTTTAAGG
AGATCAGTGAAGTGTTCCCCGACCAGTTTATTCACCTGGGCGGGGATGAGGTCGAA
TTCAAGTGTTGGGAGAGCAATCCTAAAATCCAGGACTTTATGAGACAGAAGGGATT
CGGCACCGACTTCAAGAAACTGGAATCATTCTACATTCAGAAGGTGCTGGACATCA
TTGCCACAATCAACAAAGGGAGCATCGTGTGGCAGGAGGTCTTTGACGATAAGGCC
AAACTGGCTCCCGGAACTATCGTGGAAGTCTGGAAGGATAGCGCCTATCCTGAGGA
ACTGTCACGCGTGACCGCTAGCGGCTTTCCCGTCATCCTGAGCGCACCTTGGTACCT
GGACCTGATTTCCTATGGGCAGGATTGGCGAAAGTACTATAAAGTGGAGCCCCTGG
ACTTCGGAGGCACCCAGAAGCAGAAACAGCTGTTTATCGGGGGAGAGGCCTGCCTG
TGGGGGGAATACGTGGATGCTACCAATCTGACACCACGACTGTGGCCTCGGGCTAG
CGCAGTGGGAGAGCGCCTGTGGTCTAGTAAGGACGTGCGGGATATGGACGATGCCT
ATGACCGGCTGACCCGACATCGGTGTAGAATGGTGGAACGGGGCATTGCCGCTCAG
CCACTGTATGCCGGGTACTGTAATCACGAAAATATGTGACTCGAGGCGGCCGCTCT
AGAGATCTTTTTCCCTCTGCCAAAAATTATGGGGACATCATGAAGCCCCTTGAGCAT
CTGACTTCTGGCTAATAAAGGAAATTTATTTTCATTGCAATAGTGTGTTGGAATTTTT
TGTGTCTCTCACTCGGCATGCTGGGGAGAGATCTGAGGAACCCCTAGTGATGGAGT
TGGCCACTCCCTCTCTGCGCGCTCGCTCGCTCACTGAGGCCGCCCGGGCAAAGCCCG
GGCGTCGGGCGACCTTTGGTCGCCCGGCCTCAGTGAGCGAGCGAGCGCGCAGAGAG
GGAGTGGCCATGCAGCCAGCTGGCGTAATAGCGAAGAGGCCCGCACCGATCGCCCT
TCCCAACAGTTGCGTAGCCTGAATGGCGAATGGCGCGACGCGCCCTGTAGCGGCGC
ATTAAGCGCGGCGGGTGTGGTGGTTACGCGCAGCGTGACCGCTACACTTGCCAGCG
CCCTAGCGCCCGCTCCTTTCGCTTTCTTCCCTTCCTTTCTCGCCACGTTCGCCGGCTTT
CCCCGTCAAGCTCTAAATCGGGGGCTCCCTTTAGGGTTCCGATTTAGTGCTTTACGG
CACCTCGACCCCAAAAAACTTGATTAGGGTGATGGTTCACGTAGTGGGCCATCGCC
CTGATAGACGGTTTTTCGCCCTTTGACGTTGGAGTCCACGTTCTTTAATAGTGGACT
CTTGTTCCAAACTGGAACAACACTCAACCCTATCTCGGTCTATTCTTTTGATTTATAA
GGGATTTTGCCGATTTCGGCCTATTGGTTAAAAAATGAGCTGATTTAACAAAAATTT
AACGCGAATTTTAACAAAATATTAACGTTTACAATTTCCTGATGCGGTATTTTCTCC
TTACGCATCTGTGCGGTATTTCACACCGCATATGGTGCACTCTCAGTACAATCTGCT
CTGATGCCGCATAGTTAAGCCAGCCCCGACACCCGCCAACACCCGCTGACGCGCCC
TGACGGGCTTGTCTGCTCCCGGCATCCGCTTACAGACAAGCTGTGACCGTCTCCGGG
AGCTGCATGTGTCAGAGGTTTTCACCGTCATCACCGAAACGCGCGAGACGAAAGGG
CCTCGTGATACGCCTATTTTTATAGGTTAATGTCATGATAATAATGGTTTCTTAGAC
GTCAGGTGGCACTTTTCGGGGAAATGTGCGCGGAACCCCTATTTGTTTATTTTTCTA
AATACATTCAAATATGTATCCGCTCATGAGACAATAACCCTGATAAATGCTTCAATA
ATATTGAAAAAGGAAGAGTATGAGTATTCAACATTTCCGTGTCGCCCTTATTCCCTT
TTTTGCGGCATTTTGCCTTCCTGTTTTTGCTCACCCAGAAACGCTGGTGAAAGTAAA
AGATGCTGAAGATCAGTTGGGTGCACGAGTGGGTTACATCGAACTGGATCTCAACA
GCGGTAAGATCCTTGAGAGTTTTCGCCCCGAAGAACGTTTTCCAATGATGAGCACTT
TTAAAGTTCTGCTATGTGGCGCGGTATTATCCCGTATTGACGCCGGGCAAGAGCAAC
TCGGTCGCCGCATACACTATTCTCAGAATGACTTGGTTGAGTACTCACCAGTCACAG
AAAAGCATCTTACGGATGGCATGACAGTAAGAGAATTATGCAGTGCTGCCATAACC
ATGAGTGATAACACTGCGGCCAACTTACTTCTGACAACGATCGGAGGACCGAAGGA
GCTAACCGCTTTTTTGCACAACATGGGGGATCATGTAACTCGCCTTGATCGTTGGGA
ACCGGAGCTGAATGAAGCCATACCAAACGACGAGCGTGACACCACGATGCCTGTAG
CAATGGCAACAACGTTGCGCAAACTATTAACTGGCGAACTACTTACTCTAGCTTCCC
GGCAACAATTAATAGACTGGATGGAGGCGGATAAAGTTGCAGGACCACTTCTGCGC
TCGGCCCTTCCGGCTGGCTGGTTTATTGCTGATAAATCTGGAGCCGGTGAGCGTGGG
TCTCGCGGTATCATTGCAGCACTGGGGCCAGATGGTAAGCCCTCCCGTATCGTAGTT
ATCTACACGACGGGGAGTCAGGCAACTATGGATGAACGAAATAGACAGATCGCTG
AGATAGGTGCCTCACTGATTAAGCATTGGTAACTGTCAGACCAAGTTTACTCATATA
TACTTTAGATTGATTTAAAACTTCATTTTTAATTTAAAAGGATCTAGGTGAAGATCC
TTTTTGATAATCTCATGACCAAAATCCCTTAACGTGAGTTTTCGTTCCACTGAGCGTC
AGACCCCGTAGAAAAGATCAAAGGATCTTCTTGAGATCCTTTTTTTCTGCGCGTAAT
CTGCTGCTTGCAAACAAAAAAACCACCGCTACCAGCGGTGGTTTGTTTGCCGGATC
AAGAGCTACCAACTCTTTTTCCGAAGGTAACTGGCTTCAGCAGAGCGCAGATACCA
AATACTGTCCTTCTAGTGTAGCCGTAGTTAGGCCACCACTTCAAGAACTCTGTAGCA
CCGCCTACATACCTCGCTCTGCTAATCCTGTTACCAGTGGCTGCTGCCAGTGGCGAT
AAGTCGTGTCTTACCGGGTTGGACTCAAGACGATAGTTACCGGATAAGGCGCAGCG
GTCGGGCTGAACGGGGGGTTCGTGCACACAGCCCAGCTTGGAGCGAACGACCTACA
CCGAACTGAGATACCTACAGCGTGAGCATTGAGAAAGCGCCACGCTTCCCGAAGGG
AGAAAGGCGGACAGGTATCCGGTAAGCGGCAGGGTCGGAACAGGAGAGCGCACGA
GGGAGCTTCCAGGGGGAAACGCCTGGTATCTTTATAGTCCTGTCGGGTTTCGCCACC
TCTGACTTGAGCGTCGATTTTTGTGATGCTCGTCAGGGGGGCGGAGCCTATGGAAA
AACGCCAGCAACGCGGCCTTTTTACGGTTCCTGGCCTTTTGCTGGCCTTTTGCTCAC
ATGTTCTTTCCTGCGTTATCCCCTGATTCTGTGGATAACCGTATTACCGCCTTTGAGT
GAGCTGATACCGCTCGCCGCAGCCGAACGACCGAGCGCAGCGAGTCAGTGAGCGA
GGAAGCGGAAGAGCGCCCAATACGCAAACCGCCTCTCCCCGCGCGTTGGCCGATTC
ATTAATGCAGCTGGGCTGCAGGGGGGGGGGGGGGGGGGTGGGGGGGGGGGGGGG
GGG
>SEQ ID NO: 9: AAV-hHexA-BiCBA-hHexB nucleic acid sequence
TTGGCCACTCCCTCTCTGCGCGCTCGCTCGCTCACTGAGGCCGGGCGACCAAAGGTC
GCCCGACGCCCGGGCTTTGCCCGGGCGGCCTCAGTGAGCGAGCGAGCGCGCAGAGA
GGGAGTGGCCAACTCCATCACTAGGGGTTCCTCAGATCTGAATTCTACCACATTTGT
AGAGGTTTTACTTGCTTTAAAAAACCTCCCACATCTCCCCCTGAACCTGAAACATAA
AATGAATGCAATTGTTGTTGTTAACTTGTTTATTGCAGCTTATAATGGTTACAAATA
AAGCAATAGCATCACAAATTTCACAAATAAAGCATTTTTTTCACTGCATTCTAGTTG
TGGTTTGTCCAAACTCATCAATGTATCTTATCATGTCTGTCGAGTCAGGTCTGTTCAA
ACTCCTGCTCACAGAAGCCTACATTGAGGGGTTGGGCCTGGACACCTCGCCTCAGC
AACTCACAGCGGAAGTGTGACAAACGTTCATAGGCAAATGTCAGGTCAGATGTCAA
CTTGTTGCTCCACAGCCTTTCGGCAACAGCCCCTGCTCTGGGCCAGAGCCTGGGGAC
CAGGTTTGTGTTGTCCACATATTCTCCCCACATACAAGCCTCTCCACCAATCACCAG
AGCCTTCTGCTCAGGGGTACCTTCAAATGCCAGGGGTTCCACTATGTAGAAATCCTT
CCAGTCAGGGCCATAGGATATACGGTTCAGGTACCAGGGGGCAGAGAGAAGGGCC
CGGAAGCCGGCCTTGGTGACCAGTTCCAGCTCCTTCATATAGTTCACTGGAATATCC
TCTCGCCACACCTGTATGATTGTGTCTGGCTGAATCTTTACTTTATTATCAAACACCT
CCTGCCACACCACATAGCCCTTGCCATAAGAAGAGACGATGTCCAGCAGCGTCTGG
ATGTAGAAGGACTCCAGCTGCTTGAAGTCCTCACCGAAGCCTTTCTTCCTCATAAAG
TCCTGGATCTCTGGGTTGGACTTCCAGCAGGTGAAATCAACCTCATCTCCTCCAAGA
TGAAGATAAAAATCTGGGAAGACAGAGCTGACTTCTAAGAAGAATGTGCTCATGAA
CTCATAGGTATTATTGAGACTGGGATTCACTGGTCCAAAGGTGCCAGAGGGCTCAG
ACCCAGAGTAGCAAGGAGTCAGTAATCCAGGGATACCTGGTCCCCAGGACAAAGTG
TGGCCAGGAGTGTCAAACTCTGCAAGCACACGGATACCCCGGAGCCGTGCGTATTC
AATGACCTCCTTCACATCCTGTGCTGTGTAGATGTGGGTGACAGGGTTGTAGGACCC
CTTTCTCATGAGCTCTGGAAAAGTGAAGCTCTCATATGGGAAGGAAGGATCATCTA
CCAGATGCCAGTGGAACACGTTCAATTTATTGTACGCCATGACATCCAGAGTGTCCA
GGATGCTAGAGAGTGGCAGGTAATGGCGAGATGTATCCAACAGCAAGCCCCGGTG
AGGAAAGCGGGGAAAGTCCTCAATCTCAGTCTTGTTGATAAAGAATGTGCCCTCAG
CAGATTTCCAAACAAGCTGGCTAAAAGTCTCCAGACCTCGGAGAGCTCCCCAGACA
GTCTCAGAGAGGAGTAAACACTGGTCATCATTTATGGTCAGGGTATAATTCTCCACT
GACTCCAAAGTAGGAAGCTGGTTACATCCAGGTGTGACTACAGAGACAACCAACAC
ATTCTTCTCCAGTGTATGCCGTTTCCCTGTGAGGTAAGGACGGGGCCAAGACCCGGA
ACCGAAAAGCAGGTCACGATAGCGCTGGAAGGCCTCGTCGAGGACTGAGCAGCCG
GGCTGCGCGGCCGAGCTGACATCGTACTGGAATTGAAAGTTGTTCGGGTAAAGGAC
GTAGCGCTGGTCGGAGGTTTGGAAGTTCTGAGGCCAGGGCCAGAGGGCCGTCGCCC
GTCCTGCGAACGCTGCCGCCAGCAGCAGCGAAAACCAAAGCCTGGAACTTGTCATG
GTGCTCCACTAGCGCTAGAGCTTGCTCCCGCCCGCCGCGCGCTTCGCTTTTTATAGG
GCCGCCGCCGCCGCCGCCTCGCCATAAAAGGAAACTTTCGGAGCGCGCCGCTCTGA
TTGGCTGCCGCCGCACCTCTCCGCCTCGCCCCGCCCCGCCCCTCGCCCCCATCGCTG
CACAAAATAATTAAAAAATAAATAAATACAAAATTGGGGGTGGGGAGGGGGGGGA
GATGGGGAGAGTGAAGCAGAACGTGGCCTCGGATCCCCCGGGCTGCAGTATTAATA
GTAATCAATTACGGGGTCATTAGTTCATAGCCCATATATGGAGTTCCGCGTTACATA
ACTTACGGTAAATGGCCCGCCTGGCTGACCGCCCAACGACCCCCGCCCATTGACGT
CAATAATGACGTATGTTCCCATAGTAACGCCAATAGGGACTTTCCATTGACGTCAAT
GGGTGGAGTATTTACGGTAAACTGCCCACTTGGCAGTACATCAAGTGTATCATATGC
CAAGTACGCCCCCTATTGACGTCAATGACGGTAAATGGCCCGCCTGGCATTATGCCC
AGTACATGACCTTATGGGACTTTCCTACTTGGCAGTACATCTACGTATTAGTCATCG
CTATTACCATGTCGAGGCCACGTTCTGCTTCACTCTCCCCATCTCCCCCCCCTCCCCA
CCCCCAATTTTGTATTTATTTATTTTTTAATTATTTTGTGCAGCGATGGGGGCGAGGG
GCGGGGCGGGGCGAGGCGGAGAGGTGCGGCGGCAGCCAATCAGAGCGGCGCGCTC
CGAAAGTTTCCTTTTATGGCGAGGCGGCGGCGGCGGCGGCCCTATAAAAAGCGAAG
CGCGCGGCGGGCGGGAGCAAGCTCGCTAGCACTAGTGGAGCACCATGGAGCTGTGC
GGGCTGGGGCTGCCCCGGCCGCCCATGCTGCTGGCGCTGCTGTTGGCGACACTGCT
GGCGGCGATGTTGGCGCTGCTGACTCAGGTGGCGCTGGTGGTGCAGGTGGCGGAGG
CGGCTCGGGCCCCGAGCGTCTCGGCCAAGCCGGGGCCGGCGCTGTGGCCCCTGCCG
CTCTCGGTGAAGATGACCCCGAACCTGCTGCATCTCGCCCCGGAGAACTTCTACATC
AGCCACAGCCCCAATTCCACGGCGGGCCCCTCCTGCACCCTGCTGGAGGAAGCGTT
TCGACGATATCATGGCTATATTTTTGGTTTCTACAAGTGGCATCATGAACCTGCTGA
ATTCCAGGCTAAAACCCAGGTTCAGCAACTTCTTGTCTCAATCACCCTTCAGTCAGA
GTGTGATGCTTTCCCCAACATATCTTCAGATGAGTCTTATACTTTACTTGTGAAAGA
ACCAGTGGCTGTCCTTAAGGCCAACAGAGTTTGGGGAGCATTACGAGGTTTAGAGA
CCTTTAGCCAGTTAGTTTATCAAGATTCTTATGGAACTTTCACCATCAATGAATCCA
CCATTATTGATTCTCCAAGGTTTTCTCACAGAGGAATTTTGATTGATACATCCAGAC
ATTATCTGCCAGTTAAGATTATTCTTAAAACTCTGGATGCCATGGCTTTTAATAAGT
TTAATGTTCTTCACTGGCACATAGTTGATGACCAGTCTTTCCCATATCAGAGCATCA
CTTTTCCTGAGTTAAGCAATAAAGGAAGCTATTCTTTGTCTCATGTTTATACACCAA
ATGATGTCCGTATGGTGATTGAATATGCCAGATTACGAGGAATTCGAGTCCTGCCA
GAATTTGATACCCCTGGGCATACACTATCTTGGGGAAAAGGTCAGAAAGACCTCCT
GACTCCATGTTACAGTAGACAAAACAAGTTGGACTCTTTTGGACCTATAAACCCTAC
TCTGAATACAACATACAGCTTCCTTACTACATTTTTCAAAGAAATTAGTGAGGTGTT
TCCAGATCAATTCATTCATTTGGGAGGAGATGAAGTGGAATTTAAATGTTGGGAAT
CAAATCCAAAAATTCAAGATTTCATGAGGCAAAAAGGCTTTGGCACAGATTTTAAG
AAACTAGAATCTTTCTACATTCAAAAGGTTTTGGATATTATTGCAACCATAAACAAG
GGATCCATTGTCTGGCAGGAGGTTTTTGATGATAAAGCAAAGCTTGCGCCGGGCAC
AATAGTTGAAGTATGGAAAGACAGCGCATATCCTGAGGAACTCAGTAGAGTCACAG
CATCTGGCTTCCCTGTAATCCTTTCTGCTCCTTGGTACTTAGATTTGATTAGCTATGG
ACAAGATTGGAGGAAATACTATAAAGTGGAACCTCTTGATTTTGGCGGTACTCAGA
AACAGAAACAACTTTTCATTGGTGGAGAAGCTTGTCTATGGGGAGAATATGTGGAT
GCAACTAACCTCACTCCAAGATTATGGCCTCGGGCAAGTGCTGTTGGTGAGAGACT
CTGGAGTTCCAAAGATGTCAGAGATATGGATGACGCCTATGACAGACTGACAAGGC
ACCGCTGCAGGATGGTCGAACGTGGAATAGCTGCACAACCTCTTTATGCTGGATATT
GTAACCATGAGAACATGTAACTCGAGGCGGCCGCTCTAGAGATCTTTTTCCCTCTGC
CAAAAATTATGGGGACATCATGAAGCCCCTTGAGCATCTGACTTCTGGCTAATAAA
GGAAATTTATTTTCATTGCAATAGTGTGTTGGAATTTTTTGTGTCTCTCACTCGGCAT
GCTGGGGAGAGATCTGAGGAACCCCTAGTGATGGAGTTGGCCACTCCCTCTCTGCG
CGCTCGCTCGCTCACTGAGGCCGCCCGGGCAAAGCCCGGGCGTCGGGCGACCTTTG
GTCGCCCGGCCTCAGTGAGCGAGCGAGCGCGCAGAGAGGGAGTGGCCATGCAGCC
AGCTGGCGTAATAGCGAAGAGGCCCGCACCGATCGCCCTTCCCAACAGTTGCGTAG
CCTGAATGGCGAATGGCGCGACGCGCCCTGTAGCGGCGCATTAAGCGCGGCGGGTG
TGGTGGTTACGCGCAGCGTGACCGCTACACTTGCCAGCGCCCTAGCGCCCGCTCCTT
TCGCTTTCTTCCCTTCCTTTCTCGCCACGTTCGCCGGCTTTCCCCGTCAAGCTCTAAA
TCGGGGGCTCCCTTTAGGGTTCCGATTTAGTGCTTTACGGCACCTCGACCCCAAAAA
ACTTGATTAGGGTGATGGTTCACGTAGTGGGCCATCGCCCTGATAGACGGTTTTTCG
CCCTTTGACGTTGGAGTCCACGTTCTTTAATAGTGGACTCTTGTTCCAAACTGGAAC
AACACTCAACCCTATCTCGGTCTATTCTTTTGATTTATAAGGGATTTTGCCGATTTCG
GCCTATTGGTTAAAAAATGAGCTGATTTAACAAAAATTTAACGCGAATTTTAACAA
AATATTAACGTTTACAATTTCCTGATGCGGTATTTTCTCCTTACGCATCTGTGCGGTA
TTTCACACCGCATATGGTGCACTCTCAGTACAATCTGCTCTGATGCCGCATAGTTAA
GCCAGCCCCGACACCCGCCAACACCCGCTGACGCGCCCTGACGGGCTTGTCTGCTC
CCGGCATCCGCTTACAGACAAGCTGTGACCGTCTCCGGGAGCTGCATGTGTCAGAG
GTTTTCACCGTCATCACCGAAACGCGCGAGACGAAAGGGCCTCGTGATACGCCTAT
TTTTATAGGTTAATGTCATGATAATAATGGTTTCTTAGACGTCAGGTGGCACTTTTC
GGGGAAATGTGCGCGGAACCCCTATTTGTTTATTTTTCTAAATACATTCAAATATGT
ATCCGCTCATGAGACAATAACCCTGATAAATGCTTCAATAATATTGAAAAAGGAAG
AGTATGAGTATTCAACATTTCCGTGTCGCCCTTATTCCCTTTTTTGCGGCATTTTGCC
TTCCTGTTTTTGCTCACCCAGAAACGCTGGTGAAAGTAAAAGATGCTGAAGATCAGT
TGGGTGCACGAGTGGGTTACATCGAACTGGATCTCAACAGCGGTAAGATCCTTGAG
AGTTTTCGCCCCGAAGAACGTTTTCCAATGATGAGCACTTTTAAAGTTCTGCTATGT
GGCGCGGTATTATCCCGTATTGACGCCGGGCAAGAGCAACTCGGTCGCCGCATACA
CTATTCTCAGAATGACTTGGTTGAGTACTCACCAGTCACAGAAAAGCATCTTACGGA
TGGCATGACAGTAAGAGAATTATGCAGTGCTGCCATAACCATGAGTGATAACACTG
CGGCCAACTTACTTCTGACAACGATCGGAGGACCGAAGGAGCTAACCGCTTTTTTG
CACAACATGGGGGATCATGTAACTCGCCTTGATCGTTGGGAACCGGAGCTGAATGA
AGCCATACCAAACGACGAGCGTGACACCACGATGCCTGTAGCAATGGCAACAACGT
TGCGCAAACTATTAACTGGCGAACTACTTACTCTAGCTTCCCGGCAACAATTAATAG
ACTGGATGGAGGCGGATAAAGTTGCAGGACCACTTCTGCGCTCGGCCCTTCCGGCT
GGCTGGTTTATTGCTGATAAATCTGGAGCCGGTGAGCGTGGGTCTCGCGGTATCATT
GCAGCACTGGGGCCAGATGGTAAGCCCTCCCGTATCGTAGTTATCTACACGACGGG
GAGTCAGGCAACTATGGATGAACGAAATAGACAGATCGCTGAGATAGGTGCCTCAC
TGATTAAGCATTGGTAACTGTCAGACCAAGTTTACTCATATATACTTTAGATTGATT
TAAAACTTCATTTTTAATTTAAAAGGATCTAGGTGAAGATCCTTTTTGATAATCTCA
TGACCAAAATCCCTTAACGTGAGTTTTCGTTCCACTGAGCGTCAGACCCCGTAGAAA
AGATCAAAGGATCTTCTTGAGATCCTTTTTTTCTGCGCGTAATCTGCTGCTTGCAAA
CAAAAAAACCACCGCTACCAGCGGTGGTTTGTTTGCCGGATCAAGAGCTACCAACT
CTTTTTCCGAAGGTAACTGGCTTCAGCAGAGCGCAGATACCAAATACTGTCCTTCTA
GTGTAGCCGTAGTTAGGCCACCACTTCAAGAACTCTGTAGCACCGCCTACATACCTC
GCTCTGCTAATCCTGTTACCAGTGGCTGCTGCCAGTGGCGATAAGTCGTGTCTTACC
GGGTTGGACTCAAGACGATAGTTACCGGATAAGGCGCAGCGGTCGGGCTGAACGG
GGGGTTCGTGCACACAGCCCAGCTTGGAGCGAACGACCTACACCGAACTGAGATAC
CTACAGCGTGAGCATTGAGAAAGCGCCACGCTTCCCGAAGGGAGAAAGGCGGACA
GGTATCCGGTAAGCGGCAGGGTCGGAACAGGAGAGCGCACGAGGGAGCTTCCAGG
GGGAAACGCCTGGTATCTTTATAGTCCTGTCGGGTTTCGCCACCTCTGACTTGAGCG
TCGATTTTTGTGATGCTCGTCAGGGGGGCGGAGCCTATGGAAAAACGCCAGCAACG
CGGCCTTTTTACGGTTCCTGGCCTTTTGCTGGCCTTTTGCTCACATGTTCTTTCCTGC
GTTATCCCCTGATTCTGTGGATAACCGTATTACCGCCTTTGAGTGAGCTGATACCGC
TCGCCGCAGCCGAACGACCGAGCGCAGCGAGTCAGTGAGCGAGGAAGCGGAAGAG
CGCCCAATACGCAAACCGCCTCTCCCCGCGCGTTGGCCGATTCATTAATGCAGCTGG
GCTGCAGGGGGGGGGGGGGGGGGGTGGGGGGGGGGGGGGGGGG

What is claimed is:

1. An isolated nucleic acid construct, comprising
(i) a first expression cassette, comprising a nucleic acid encoding a hexosaminidase alpha-subunit (HEXA) under the control of a first promoter, and
(ii) a second expression cassette, comprising a nucleic acid encoding a hexosaminidase beta-subunit (HEXB) under the control of a second promoter,
wherein the first expression cassette and the second expression cassette are flanked by adeno-associated virus (AAV) inverted terminal repeats (ITRs).

2. The isolated nucleic acid of claim 1, wherein a first intron is present between the first promoter and the sequence of the nucleic acid encoding the hexosaminidase alpha-subunit (HEXA).

3. The isolated nucleic acid of claim 1 or 2, wherein a second intron is present between the second promoter and the sequence of the nucleic acid encoding the hexosaminidase beta-subunit (HEXB).

4. The isolated nucleic acid construct of any one of claims 1 to 3, wherein the HexA comprises an amino acid sequence as set forth in SEQ ID NO: 1.

5. The isolated nucleic acid construct of any one of claims 1 to 4, wherein the HexB comprises an amino acid sequence as set forth in SEQ ID NO: 2.

6. The isolated nucleic acid construct of any one of claims 1 to 5, wherein the first intron is positioned between the first promoter and the nucleic acid sequence encoding the HexA, optionally wherein the first intron is a chimeric intron.

7. The isolated nucleic acid construct of any one of claims 1 to 6, wherein the first promoter is positioned proximal to an AAV ITR, optionally wherein the first promoter is positioned between an AAV ITR and a nucleic acid sequence encoding HexA.

8. The isolated nucleic acid construct of any one of claims 1 to 7, wherein the first promoter and/or the second promoter is a P2 promoter.

9. The isolated nucleic acid construct of any one of claims 1 to 8, wherein the second intron is positioned between the second promoter and the nucleic acid sequence encoding the HexB, optionally wherein the second intron is a chimeric intron.

10. The isolated nucleic acid construct of any one of claims 1 to 9, wherein the second promoter is positioned proximal to an AAV ITR, optionally wherein the second promoter is positioned between an AAV ITR and a nucleic acid sequence encoding HexB.

11. The isolated nucleic acid construct of any one of claims 1 to 10, wherein the first expression cassette comprises a first poly A signal operably linked to the nucleic acid sequence encoding HexA, optionally wherein the first poly A signal is a BGH poly A signal.

12. The isolated nucleic acid construct of any one of claims 1 to 11, wherein the second expression cassette comprises a second poly A signal operably linked to the nucleic acid sequence encoding HexB, optionally wherein the second poly A tail is an SV40 poly A signal.

13. The isolated nucleic acid construct of any one of claims 1 to 12, wherein the first poly A signal and the second poly A signal are positioned adjacent to one another.

14. An isolated nucleic acid construct, comprising
(i) a first expression cassette, comprising a nucleic acid encoding a hexosaminidase alpha-subunit, and
(ii) a second expression cassette, comprising a nucleic acid encoding a hexosaminidase beta-subunit,
wherein the first expression cassette and the second expression cassette are operably linked by a bidirectional promoter, and wherein the first expression cassette and the second expression cassette are flanked by adeno-associated virus (AAV) inverted terminal repeats (ITRs).

15. The isolated nucleic acid construct of claim 14, wherein the HexA comprises an amino acid sequence as set forth in SEQ ID NO: 1.

16. The isolated nucleic acid construct of claim 14 or 15, wherein the HexB comprises an amino acid sequence as set forth in SEQ ID NO: 2.

17. The isolated nucleic acid construct of any one of claims 14 to 16, wherein the bidirectional promoter comprises at least one chicken beta-actin (CBA) promoter.

18. The isolated nucleic acid construct of claim 17, wherein the bidirectional promoter comprises two CBA promoters, wherein the CBA promoters initiate transcription of the first expression cassette and the second expression cassette in opposite directions.

19. The isolated nucleic acid construct of claim 18, wherein the bidirectional promoter comprises a CMV enhancer sequence, optionally wherein the CMV enhancer sequence is positioned between the two CBA promoters.

20. The isolated nucleic acid construct of any one of claims 14 to 19, wherein the first expression construct comprises a first poly A signal, optionally wherein the first poly A signal is proximal to an AAV ITR.

21. The isolated nucleic acid construct of any one of claims 14 to 20, wherein the first expression construct comprises a second poly A signal, optionally wherein the second poly A signal is proximal to an AAV ITR.

22. The isolated nucleic acid construct of any one of claims 14 to 21, wherein the first and/or second poly A signal is each selected from SV40 poly A signal, rabbit beta-globulin (RBG) poly A signal, and bovine growth hormone (BGH) poly A signal.

23. An isolated nucleic acid comprising the sequence set forth in any one of SEQ ID NOs: 3-9.

24. A recombinant AAV (rAAV) comprising:
(i) a capsid protein; and
(ii) an isolated nucleic acid of any one of claims 1 to 23.

25. The rAAV of claim 24, wherein the capsid protein is of a serotype selected from AAV1, AAV2, AAV3, AAV4, AAV5, AAV6, AAV7, AAV8, AAVrh8, AAV9, AAV10, AAVrh10, and AAV.PHP.B.

26. The rAAV of claim 24 or 25, wherein the isolated nucleic acid comprises an ITR selected from the group consisting of AAV1 ITR, AAV2 ITR, AAV3 ITR, AAV4 ITR, AAV5 ITR, or AAV6 ITR.

27. A host cell comprising the isolated nucleic acid of any one of claims 1 to 23, or the rAAV of any one of claims 22 to 24.

28. The host cell of claim 27, wherein the host cell is a mammalian cell, yeast cell, bacterial cell, insect cell, plant cell, or fungal cell.

29. A method for treating a lysosomal storage disease, the method comprising administering the isolated nucleic acid of any one of claims 1 to 23, or the rAAV of any one of claims 24 to 26, to a subject having or suspected of having a lysosomal storage disease.

30. The method of claim 29, wherein the lysosomal storage disease is Tay-Sachs disease or Sandhoff disease.

31. The method of claim 29 or 30, wherein the subject is characterized as having a mutation in a HEXA gene resulting in reduced or loss of function of a hexosaminidase alpha-subunit of the subject.

32. The method of claim 29 or 30, wherein the subject is characterized as having a mutation in a HEXB gene resulting in reduced or loss of function of a hexosaminidase beta-subunit of the subject.

33. The method of any one of claims 29 to 32, wherein the rAAV is administered by intracranial injection, intracerebral injection, or injection into the CSF via the cerebral ventricular system, cisterna magna, or intrathecal space.

34. The method of any one of claims 29 to 33, wherein the subject is administered the isolated nucleic acid or the rAAV during a pre-symptomatic stage of the lysosomal storage disease.

35. The method of claim 34, wherein the pre-symptomatic stage of the lysosomal storage disease occurs between birth (e.g., perinatal) and 4-weeks of age.


SEQUENCE LISTING (9 sequences)

SEQ ID NO: 1 - protein, 529 aa, synthetic construct
  REGION 1..529: note = Synthetic polypeptide
MTSSRLWFSLLLAAAFAGRATALWPWPQNFQTSDQRYVLYPNNFQFQYDVSSAAQPGCSVLDEAFQRYRDLLFGSGSWPRPYLTGKRHTLEKNVLVVSVVTPGCNQLPTLESVENYTLTINDDQCLLLSETVWGALRGLETFSQLVWKSAEGTFFINKTEIEDFPRFPHRGLLLDTSRHYLPLSSILDTLDVMAYNKLNVFHWHLVDDPSFPYESFTFPELMRKGSYNPVTHIYTAQDVKEVIEYARLRGIRVLAEFDTPGHTLSWGPGIPGLLTPCYSGSEPSGTFGPVNPSLNNTYEFMSTFFLEVSSVFPDFYLHLGGDEVDFTCWKSNPEIQDFMRKKGFGEDFKQLESFYIQTLLDIVSSYGKGYVVWQEVFDNKVKIQPDTIIQVWREDIPVNYMKELELVTKAGFRALLSAPWYLNRISYGPDWKDFYVVEPLAFEGTPEQKALVIGGEACMWGEYVDNTNLVPRLWPRAGAVAERLWSNKLTSDLTFAYERLSHFRCELLRRGVQAQPLNVGFCEQEFEQT

SEQ ID NO: 2 - protein, 556 aa, synthetic construct
  REGION 1..556: note = Synthetic polypeptide
MELCGLGLPRPPMLLALLLATLLAAMLALLTQVALVVQVAEAARAPSVSAKPGPALWPLPLSVKMTPNLLHLAPENFYISHSPNSTAGPSCTLLEEAFRRYHGYIFGFYKWHHEPAEFQAKTQVQQLLVSITLQSECDAFPNISSDESYTLLVKEPVAVLKANRVWGALRGLETFSQLVYQDSYGTFTINESTIIDSPRFSHRGILIDTSRHYLPVKIILKTLDAMAFNKFNVLHWHIVDDQSFPYQSITFPELSNKGSYSLSHVYTPNDVRMVIEYARLRGIRVLPEFDTPGHTLSWGKGQKDLLTPCYSRQNKLDSFGPINPTLNTTYSFLTTFFKEISEVFPDQFIHLGGDEVEFKCWESNPKIQDFMRQKGFGTDFKKLESFYIQKVLDIIATINKGSIVWQEVFDDKAKLAPGTIVEVWKDSAYPEELSRVTASGFPVILSAPWYLDLISYGQDWRKYYKVEPLDFGGTQKQKQLFIGGEACLWGEYVDATNLTPRLWPRASAVGERLWSSKDVRDMDDAYDRLTRHRCRMVERGIAAQPLYAGYCNHENM

SEQ ID NO: 3 - DNA, 7681 bp, synthetic construct
  misc_feature 1..7681: note = Synthetic polynucleotide
  source 1..7681: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattctaccacatttgtagaggttttacttgctttaaaaaacctcccacatctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtcgaggcggccgctcaggtctgctcaaattcctgctcacagaatccgacattcaggggctgtgcctgcacgcctctcctcagcagctcgcacctgaagtggctcagcctttcgtaagcgaatgtcagatcagaggtcagcttgttggaccacagtctctcggccactgctccggccctgggccacagtcgtgggaccagattggtgttgtccacatactcgccccacatacatgcttcgcccccgatgaccagagctttctgctcaggagtcccttcaaaggccaggggctccacaatgtagaaatccttccagtctggtccatatgagatcctgttcaggtaccaaggggcactcagcagtgctcggaagccagcttttgtcaccagttccagctccttcatataattgacaggaatatcctcgcgccacacttgaatgatggtatcgggctggattttgaccttgttgtcaaacacttcctgccagaccacatagccctttccgtatgaactcacaatgtccagcagagtctggatgtagaaactctccagctgcttgaagtcttcgccgaaccctttcttccgcataaagtcctggatctctggattagacttccaacatgtgaaatccacttcgtctccgcccaggtgcagataaaagtcagggaagacagaggacacctccagaaagaatgtagacatgaactcgtaggtattgttcagggatgggttcacaggtccgaaggtgccgcttggctcactcccagagtagcaaggagtcagcagtccgggaattcctgggccccagctcagagtatggccaggtgtatcgaattcagccagcactcgaatccccctcagtcgggcatactcgatgacttctttcacgtcctgggcggtatagatgtgagtcacgggattgtagctgcccttgcgcatcagctctggaaatgtgaagctttcgtaaggaaatgagggatcgtcgaccagatgccagtggaacacgttcagcttgttataagccatcacatccagagtgtccagaatgcttgacaggggcaggtaatgtctagatgtatccagcagcagcccccggtgtggaaatctagggaagtcttcgatctcagttttgttgataaagaatgttccctcggctgacttccagaccagctgactgaaagtctccaggcccctcagtgccccccacactgtttcggacagcagcagacactgatcgtcattgattgtcagggtatagttctccacggattccagggtgggcagctgattgcagcctggagtgaccacgctgaccaccagcacgtttttctccagggtgtggcgcttcccagtcagatagggccgtggccaggacccgcttccaaacagcagatcgcggtaccgctggaatgcctcgtccaggacgctgcatcctggctgagcggcgctgctcacgtcgtactgaaactggaaattgtttggatacagcacgtacctctggtcagatgtctggaagttctgaggccatggccacagggcggttgctcgtccagcaaatgcagcggccagcagcagggagaaccacagtctggagctggtcatggtggctttatcgatactagcgctagagcttgctcccgcccgccgcgcgcttcgctttttatagggccgccgccgccgccgcctcgccataaaaggaaactttcggagcgcgccgctctgattggctgccgccgcacctctccgcctcgccccgccccgcccctcgcccccatcgctgcacaaaataattaaaaaataaataaatacaaaattgggggtggggaggggggggagatggggagagtgaagcagaacgtggcctcggatcccccgggctgcagtattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagctcgctagcactagtggagcaccatggagctgtgcgggctggggctgccccggccgcccatgctgctggcgctgctgttggcgacactgctggcggcgatgttggcgctgctgactcaggtggcgctggtggtgcaggtggcggaggcggctcgggccccgagcgtctcggccaagccggggccggcgctgtggcccctgccgctctcggtgaagatgaccccgaacctgctgcatctcgccccggagaacttctacatcagccacagccccaattccacggcgggcccctcctgcaccctgctggaggaagcgtttcgacgatatcatggctatatttttggtttctacaagtggcatcatgaacctgctgaattccaggctaaaacccaggttcagcaacttcttgtctcaatcacccttcagtcagagtgtgatgctttccccaacatatcttcagatgagtcttatactttacttgtgaaagaaccagtggctgtccttaaggccaacagagtttggggagcattacgaggtttagagacctttagccagttagtttatcaagattcttatggaactttcaccatcaatgaatccaccattattgattctccaaggttttctcacagaggaattttgattgatacatccagacattatctgccagttaagattattcttaaaactctggatgccatggcttttaataagtttaatgttcttcactggcacatagttgatgaccagtctttcccatatcagagcatcacttttcctgagttaagcaataaaggaagctattctttgtctcatgtttatacaccaaatgatgtccgtatggtgattgaatatgccagattacgaggaattcgagtcctgccagaatttgatacccctgggcatacactatcttggggaaaaggtcagaaagacctcctgactccatgttacagtagacaaaacaagttggactcttttggacctataaaccctactctgaatacaacatacagcttccttactacatttttcaaagaaattagtgaggtgtttccagatcaattcattcatttgggaggagatgaagtggaatttaaatgttgggaatcaaatccaaaaattcaagatttcatgaggcaaaaaggctttggcacagattttaagaaactagaatctttctacattcaaaaggttttggatattattgcaaccataaacaagggatccattgtctggcaggaggtttttgatgataaagcaaagcttgcgccgggcacaatagttgaagtatggaaagacagcgcatatcctgaggaactcagtagagtcacagcatctggcttccctgtaatcctttctgctccttggtacttagatttgattagctatggacaagattggaggaaatactataaagtggaacctcttgattttggcggtactcagaaacagaaacaacttttcattggtggagaagcttgtctatggggagaatatgtggatgcaactaacctcactccaagattatggcctcgggcaagtgctgttggtgagagactctggagttccaaagatgtcagagatatggatgacgcctatgacagactgacaaggcaccgctgcaggatggtcgaacgtggaatagctgcacaacctctttatgctggatattgtaaccatgagaacatgtaactcgaggcggccgctctagagatctttttccctctgccaaaaattatggggacatcatgaagccccttgagcatctgacttctggctaataaaggaaatttattttcattgcaatagtgtgttggaattttttgtgtctctcactcggcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccatgcagccagctggcgtaatagcgaagaggcccgcaccgatcgcccttcccaacagttgcgtagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctctattctcagaatgacttggttgagtactcaccagtcacagaaaagcatcttacggatggcatgacgtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgggctgcaggggggggggggggggggtgggggggggggggggggg

SEQ ID NO: 4 - DNA, 7676 bp, synthetic construct
  misc_feature 1..7676: note = Synthetic polynucleotide
  source 1..7676: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattctaccacatttgtagaggttttacttgctttaaaaaacctcccacatctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtcgaggcggccgctcaggtctgctcaaattcctgctcacagaatccgacattcaggggctgtgcctgcacgcctctcctcagcagctcgcacctgaagtggctcagcctttcgtaagcgaatgtcagatcagaggtcagcttgttggaccacagtctctcggccactgctccggccctgggccacagtcgtgggaccagattggtgttgtccacatactcgccccacatacatgcttcgcccccgatgaccagagctttctgctcaggagtcccttcaaaggccaggggctccacaatgtagaaatccttccagtctggtccatatgagatcctgttcaggtaccaaggggcactcagcagtgctcggaagccagcttttgtcaccagttccagctccttcatataattgacaggaatatcctcgcgccacacttgaatgatggtatcgggctggattttgaccttgttgtcaaacacttcctgccagaccacatagccctttccgtatgaactcacaatgtccagcagagtctggatgtagaaactctccagctgcttgaagtcttcgccgaaccctttcttccgcataaagtcctggatctctggattagacttccaacatgtgaaatccacttcgtctccgcccaggtgcagataaaagtcagggaagacagaggacacctccagaaagaatgtagacatgaactcgtaggtattgttcagggatgggttcacaggtccgaaggtgccgcttggctcactcccagagtagcaaggagtcagcagtccgggaattcctgggccccagctcagagtatggccaggtgtatcgaattcagccagcactcgaatccccctcagtcgggcatactcgatgacttctttcacgtcctgggcggtatagatgtgagtcacgggattgtagctgcccttgcgcatcagctctggaaatgtgaagctttcgtaaggaaatgagggatcgtcgaccagatgccagtggaacacgttcagcttgttataagccatcacatccagagtgtccagaatgcttgacaggggcaggtaatgtctagatgtatccagcagcagcccccggtgtggaaatctagggaagtcttcgatctcagttttgttgataaagaatgttccctcggctgacttccagaccagctgactgaaagtctccaggcccctcagtgccccccacactgtttcggacagcagcagacactgatcgtcattgattgtcagggtatagttctccacggattccagggtgggcagctgattgcagcctggagtgaccacgctgaccaccagcacgtttttctccagggtgtggcgcttcccagtcagatagggccgtggccaggacccgcttccaaacagcagatcgcggtaccgctggaatgcctcgtccaggacgctgcatcctggctgagcggcgctgctcacgtcgtactgaaactggaaattgtttggatacagcacgtacctctggtcagatgtctggaagttctgaggccatggccacagggcggttgctcgtccagcaaatgcagcggccagcagcagggagaaccacagtctggagctggtcatggtggctttatcgatactagcgctagagcttgctcccgcccgccgcgcgcttcgctttttatagggccgccgccgccgccgcctcgccataaaaggaaactttcggagcgcgccgctctgattggctgccgccgcacctctccgcctcgccccgccccgcccctcgcccccatcgctgcacaaaataattaaaaaataaataaatacaaaattgggggtggggaggggggggagatggggagagtgaagcagaacgtggcctcggatcccccgggctgcagtattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagctcgctagcactagtggagcaccatggagctgtgcgggctggggctgccccggccgcccatgctgctggcgctgctgttggcgacactgctggcggcgatgttggcgctgctgactcaggtggcgctggtggtgcaggtggcggaggcggctcgggccccgagcgtctcggccaagccggggccggcgctgtggcccctgccgctctcggtgaagatgaccccgaacctgctgcatctcgccccggagaacttctacatcagccacagccccaattccacggcgggcccctcctgcaccctgctggaggaagcgtttcgacgatatcatggctatatttttggtttctacaagtggcatcatgaacctgctgaattccaggctaaaacccaggttcagcaacttcttgtctcaatcacccttcagtcagagtgtgatgctttccccaacatatcttcagatgagtcttatactttacttgtgaaagaaccagtggctgtccttaaggccaacagagtttggggagcattacgaggtttagagacctttagccagttagtttatcaagattcttatggaactttcaccatcaatgaatccaccattattgattctccaaggttttctcacagaggaattttgattgatacatccagacattatctgccagttaagattattcttaaaactctggatgccatggcttttaataagtttaatgttcttcactggcacatagttgatgaccagtctttcccatatcagagcatcacttttcctgagttaagcaataaaggaagctattctttgtctcatgtttatacaccaaatgatgtccgtatggtgattgaatatgccagattacgaggaattcgagtcctgccagaatttgatacccctgggcatacactatcttggggaaaaggtcagaaagacctcctgactccatgttacagtagacaaaacaagttggactcttttggacctataaaccctactctgaatacaacatacagcttccttactacatttttcaaagaaattagtgaggtgtttccagatcaattcattcatttgggaggagatgaagtggaatttaaatgttgggaatcaaatccaaaaattcaagatttcatgaggcaaaaaggctttggcacagattttaagaaactagaatctttctacattcaaaaggttttggatattattgcaaccataaacaagggatccattgtctggcaggaggtttttgatgataaagcaaagcttgcgccgggcacaatagttgaagtatggaaagacagcgcatatcctgaggaactcagtagagtcacagcatctggcttccctgtaatcctttctgctccttggtacttagatttgattagctatggacaagattggaggaaatactataaagtggaacctcttgattttggcggtactcagaaacagaaacaacttttcattggtggagaagcttgtctatggggagaatatgtggatgcaactaacctcactccaagattatggcctcgggcaagtgctgttggtgagagactctggagttccaaagatgtcagagatatggatgacgcctatgacagactgacaaggcaccgctgcaggatggtcgaacgtggaatagctgcacaacctctttatgctggatattgtaaccatgagaacatgtaactcgaggcggccgctctagagatctttttccctctgccaaaaattatggggacatcatgaagccccttgagcatctgacttctggctaataaaggaaatttattttcattgcaatagtgtgttggaattttttgtgtctctcactcggcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccatgcagccagctggcgtaatagcgaagaggcccgcaccgatcgcccttcccaacagttgcgtagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattcagaatgacttggtgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggccttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattatgcagctgggctgcaggggggggggggggggggtgggggggggggggggggg

SEQ ID NO: 5 - DNA, 7741 bp, synthetic construct
  misc_feature 1..7741: note = Synthetic polynucleotide
  source 1..7741: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattctaccacatttgtagaggttttacttgctttaaaaaacctcccacatctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtcgaggcggccgctcaggtctgctcaaattcctgctcacagaatccgacattcaggggctgtgcctgcacgcctctcctcagcagctcgcacctgaagtggctcagcctttcgtaagcgaatgtcagatcagaggtcagcttgttggaccacagtctctcggccactgctccggccctgggccacagtcgtgggaccagattggtgttgtccacatactcgccccacatacatgcttcgcccccgatgaccagagctttctgctcaggagtcccttcaaaggccaggggctccacaatgtagaaatccttccagtctggtccatatgagatcctgttcaggtaccaaggggcactcagcagtgctcggaagccagcttttgtcaccagttccagctccttcatataattgacaggaatatcctcgcgccacacttgaatgatggtatcgggctggattttgaccttgttgtcaaacacttcctgccagaccacatagccctttccgtatgaactcacaatgtccagcagagtctggatgtagaaactctccagctgcttgaagtcttcgccgaaccctttcttccgcataaagtcctggatctctggattagacttccaacatgtgaaatccacttcgtctccgcccaggtgcagataaaagtcagggaagacagaggacacctccagaaagaatgtagacatgaactcgtaggtattgttcagggatgggttcacaggtccgaaggtgccgcttggctcactcccagagtagcaaggagtcagcagtccgggaattcctgggccccagctcagagtatggccaggtgtatcgaattcagccagcactcgaatccccctcagtcgggcatactcgatgacttctttcacgtcctgggcggtatagatgtgagtcacgggattgtagctgcccttgcgcatcagctctggaaatgtgaagctttcgtaaggaaatgagggatcgtcgaccagatgccagtggaacacgttcagcttgttataagccatcacatccagagtgtccagaatgcttgacaggggcaggtaatgtctagatgtatccagcagcagcccccggtgtggaaatctagggaagtcttcgatctcagttttgttgataaagaatgttccctcggctgacttccagaccagctgactgaaagtctccaggcccctcagtgccccccacactgtttcggacagcagcagacactgatcgtcattgattgtcagggtatagttctccacggattccagggtgggcagctgattgcagcctggagtgaccacgctgaccaccagcacgtttttctccagggtgtggcgcttcccagtcagatagggccgtggccaggacccgcttccaaacagcagatcgcggtaccgctggaatgcctcgtccaggacgctgcatcctggctgagcggcgctgctcacgtcgtactgaaactggaaattgtttggatacagcacgtacctctggtcagatgtctggaagttctgaggccatggccacagggcggttgctcgtccagcaaatgcagcggccagcagcagggagaaccacagtctggagctggtcatggtggctttatcgatactagcgctagagcttgctcccgcccgccgcgcgcttcgctttttatagggccgccgccgccgccgcctcgccataaaaggaaactttcggagcgcgccgctctgattggctgccgccgcacctctccgcctcgccccgccccgcccctcgcccccatcgctgcacaaaataattaaaaaataaataaatacaaaattgggggtggggaggggggggagatggggagagtgaagcagaacgtggcctcggatcccccgggctgcagtattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagctcgctagcactagtaaagccaccatggaactgtgcggactgggactgcctagaccccctatgctgctggctctgctgctggctaccctgctggctgctatgctggccctgctgactcaggtggccctggtggtccaggtcgcagaggcagctagggctccttcagtgagcgcaaagccaggacctgccctgtggccactgccactgtccgtgaaaatgacaccaaacctgctgcacctggcccccgaaaatttctacatctcccattctcctaacagtacagcagggccatcatgcactctgctggaggaagccttccggagataccacgggtatattttcggcttctacaagtggcaccatgagcccgcagaatttcaggccaaaacccaggtgcagcagctgctggtcagcatcacactgcagtccgagtgtgacgctttcccaaatattagctccgatgagagctacaccctgctggtgaaggaacccgtggctgtcctgaaagcaaacagagtctggggcgcactgagggggctggagacattctcccagctggtgtaccaggactcttacggaacatttactatcaacgaatccactatcatcgactcccctaggttttctcaccgcggcatcctgattgatacttctcgccattacctgccagtgaagatcattctgaaaaccctggatgcaatggccttcaacaagtttaatgtgctgcactggcatatcgtcgacgatcagtctttcccttaccagagtattacatttccagagctgagtaataaaggcagttactcactgagccacgtgtatactccaaacgacgtgagaatggtcatcgagtatgccaggctgcgcggaattagggtgctgccagaattcgatactcccggccataccctgtcttggggcaaggggcagaaagacctgctgaccccctgctacagtcggcagaacaagctggattcattcggccctatcaacccaacactgaataccacatactcttttctgactaccttctttaaggagatcagtgaagtgttccccgaccagtttattcacctgggcggggatgaggtcgaattcaagtgttgggagagcaatcctaaaatccaggactttatgagacagaagggattcggcaccgacttcaagaaactggaatcattctacattcagaaggtgctggacatcattgccacaatcaacaaagggagcatcgtgtggcaggaggtctttgacgataaggccaaactggctcccggaactatcgtggaagtctggaaggatagcgcctatcctgaggaactgtcacgcgtgaccgctagcggctttcccgtcatcctgagcgcaccttggtacctggacctgatttcctatgggcaggattggcgaaagtactataaagtggagcccctggacttcggaggcacccagaagcagaaacagctgtttatcgggggagaggcctgcctgtggggggaatacgtggatgctaccaatctgacaccacgactgtggcctcgggctagcgcagtgggagagcgcctgtggtctagtaaggacgtgcgggatatggacgatgcctatgaccggctgacccgacatcggtgtagaatggtggaacggggcattgccgctcagccactgtatgccgggtactgtaatcacgaaaatatgtgactcgaggcggccgctctagagatctttttccctctgccaaaaattatggggacatcatgaagccccttgagcatctgacttctggctaataaaggaaatttattttcattgcaatagtgtgttggaattttttgtgtctctcactcggcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccatgcagccagctggcgtaatagcgaagaggcccgcaccgatcgcccttcccaacagttgcgtagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggcctcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcagagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgggctgcaggggggggggggggggggtgggggggggggggggggg

SEQ ID NO: 6 - DNA, 7090 bp, synthetic construct
  misc_feature 1..7090: note = Synthetic polynucleotide
  source 1..7090: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcctggagccggtgtcaggttgctccggtaacggtgacgtgcacgcgtgggcggagccatcacgcaggttgctatataagcagagctcgtttagtgaaccgtcagaatcgatccaccatggccggctgcaggctctgggtttcgctgctgctggcggcggcgttggcttgcttggccacggcactgtggccgtggccccagtacatccaaacctaccaccggcgctacaccctgtaccccaacaacttccagttccggtaccatgtcagttcggccgcgcaggcgggctgcgtcgtcctcgacgaggcctttcgacgctaccgtaacctgctcttcggttccggctcttggccccgacccagcttctcaaataaacagcaaacgttggggaagaacattctggtggtctccgtcgtcacagctgaatgtaatgaatttcctaatttggagtcggtagaaaattacaccctaaccattaatgatgaccagtgtttactcgcctctgagactgtctggggcgctctccgaggtctggagactttcagtcagcttgtttggaaatcagctgagggcacgttctttatcaacaagacaaagattaaagactttcctcgattccctcaccggggcgtactgctggatacatctcgccattacctgccattgtctagcatcctggatacactggatgtcatggcatacaataaattcaacgtgttccactggcacttggtggacgactcttccttcccatatgagagcttcactttcccagagctcaccagaaaggggtccttcaaccctgtcactcacatctacacagcacaggatgtgaaggaggtcattgaatacgcaaggcttcggggtatccgtgtgctggcagaatttgacactcctggccacactttgtcctgggggccaggtgcccctgggttattaacaccttgctactctgggtctcatctctctggcacatttggaccggtgaaccccagtctcaacagcacctatgacttcatgagcacactcttcctggagatcagctcagtcttcccggacttttatctccacctgggaggggatgaagtcgacttcacctgctggaagtccaaccccaacatccaggccttcatgaagaaaaagggctttactgacttcaagcagctggagtccttctacatccagacgctgctggacatcgtctctgattatgacaagggctatgtggtgtggcaggaggtatttgataataaagtgaaggttcggccagatacaatcatacaggtgtggcgggaagaaatgccagtagagtacatgttggagatgcaagatatcaccagggctggcttccgggccctgctgtctgctccctggtacctgaaccgtgtaaagtatggccctgactggaaggacatgtacaaagtggagcccctggcgtttcatggtacgcctgaacagaaggctctggtcattggaggggaggcctgtatgtggggagagtatgtggacagcaccaacctggtccccagactctggcccagagcgggtgccgtcgctgagagactgtggagcagtaacctgacaactaatatagactttgcctttaaacgtttgtcgcatttccgttgtgagctggtgaggagaggaatccaggcccagcccatcagtgtaggctactgtgagcaggagtttgagcagacttgagcggccgcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcagtaccacatttgtagaggttttacttgctttaaaaaacctcccacatctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgctcgacctcgagctatattttattctcatagttacagtatccagtatagagaggttgtgcagctattccacggctgaccattctgcagcggtgcacggccagtcgtttgtaggcattttctaggtcagtgacagttttagggctccagagtctctcaccaacagcgcttgctcgaggccataatcttggagtaaggttagttgcatccacaaattctccccacaggcaagcttctccaccaataacaagttgtttctgcttctcagagccttcaaaattaaggggctcaactttgtagtagtttttccagtcttgcccatagctgatcaggtctaagtaccaaggagcagaaaggatggcagggaagccagagcctgtgacttgctttagctcatatgaataatgctcactcttccacacttcgactactgtgcccggctgaagctccaccttatcatcaaaaacttcttgccaaacaatggagttcttctttaaggatgaaataatttccaaaatctttttaatataaaaggattctagtcttctaaaatcgctgccaaagccctttctcttcatgaaaccttggatgtttggatttgatgcccaacattgaaattctacttcatctcctcccaagtggatgaactgatctggaaacacactgctgatttctttgaaaaatgtgttaaagaatgcatacgttgtgtttacagttgggtctacaggcccaaacacttgagttttagttttttgattgtaacatggagttagaaggtttttctgtcctttgccccaagactgtgtatggccaggggtatcaaattctggtatgactcgaatccctcggagccgggcgtactccagcaccatccggacatcgtttggtgtatagacatgagacaaagagtagcttcccttattgcttagctcaggaaaagtggtactctgataagggaaagactggtcgtccactatgtgccagtgaagaacattaaacttattaaaagccatggcatccagagtttttaaaattgtcttcacaggcaggaagtgtctagatgtatcaattaaaattcctctatgagggaatcttggagaatcagctatactggattcattgatggtgaaagtcccgaaagagtcttggtaaactaactggctaaacgtctctaaacctcgtaacgctccccaaacgctgttggccttgaggacggctactggttcttgtacaagcagagaataggtttcatctgaagacagactagggaaggactcgcactctgactcgagggtaatggagaccaggagcttctgcaactgtggctcagctcgaaatctagcagggccatgatgtctcttgtagaaaccaaaaacatagttgtaatatcgccgaaacgcctcctgtagcagcgagcaggaagggcccgctgtggaattgggactgtggtcgatgctgaagtcctccgcggagatgtacaacagccgcgggaacatctgcaccgagcgcgggaagggccatagcgcaggttgcagtcgggccggggccactagggccagcgacactagcgacaccagcgcctgcagcagcagcagcccgggggcgctacgcggggactgcggcatggtggactagttctgacggttcactaaacgagctctgcttatatagcaacctgcgtgatggctccgcccacgcgtgcacgtcaccgttaccggagcaacctgacaccggctccaggcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccatgcagccagctggcgtaatagcgaagaggcccgcaccgatcgcccttcccaacagttgcgtagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgggctgcaggggggggggggggggggtgggggggggggggggggg

SEQ ID NO: 7 - DNA, 7743 bp, synthetic construct
  misc_feature 1..7743: note = Synthetic polynucleotide
  source 1..7743: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattctaccacatttgtagaggttttacttgctttaaaaaacctcccacatctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtcgaggcggccgctcaggtctgctcaaattcctgctcacagaatccgacattcaggggctgtgcctgcacgcctctcctcagcagctcgcacctgaagtggctcagcctttcgtaagcgaatgtcagatcagaggtcagcttgttggaccacagtctctcggccactgctccggccctgggccacagtcgtgggaccagattggtgttgtccacatactcgccccacatacatgcttcgcccccgatgaccagagctttctgctcaggagtcccttcaaaggccaggggctccacaatgtagaaatccttccagtctggtccatatgagatcctgttcaggtaccaaggggcactcagcagtgctcggaagccagcttttgtcaccagttccagctccttcatataattgacaggaatatcctcgcgccacacttgaatgatggtatcgggctggattttgaccttgttgtcaaacacttcctgccagaccacatagccctttccgtatgaactcacaatgtccagcagagtctggatgtagaaactctccagctgcttgaagtcttcgccgaaccctttcttccgcataaagtcctggatctctggattagacttccaacatgtgaaatccacttcgtctccgcccaggtgcagataaaagtcagggaagacagaggacacctccagaaagaatgtagacatgaactcgtaggtattgttcagggatgggttcacaggtccgaaggtgccgcttggctcactcccagagtagcaaggagtcagcagtccgggaattcctgggccccagctcagagtatggccaggtgtatcgaattcagccagcactcgaatccccctcagtcgggcatactcgatgacttctttcacgtcctgggcggtatagatgtgagtcacgggattgtagctgcccttgcgcatcagctctggaaatgtgaagctttcgtaaggaaatgagggatcgtcgaccagatgccagtggaacacgttcagcttgttataagccatcacatccagagtgtccagaatgcttgacaggggcaggtaatgtctagatgtatccagcagcagcccccggtgtggaaatctagggaagtcttcgatctcagttttgttgataaagaatgttccctcggctgacttccagaccagctgactgaaagtctccaggcccctcagtgccccccacactgtttcggacagcagcagacactgatcgtcattgattgtcagggtatagttctccacggattccagggtgggcagctgattgcagcctggagtgaccacgctgaccaccagcacgtttttctccagggtgtggcgcttcccagtcagatagggccgtggccaggacccgcttccaaacagcagatcgcggtaccgctggaatgcctcgtccaggacgctgcatcctggctgagcggcgctgctcacgtcgtactgaaactggaaattgtttggatacagcacgtacctctggtcagatgtctggaagttctgaggccatggccacagggcggttgctcgtccagcaaatgcagcggccagcagcagggagaaccacagtctggagctggtcatggtggctttatcgatactagcgctagagcttgctcccgcccgccgcgcgcttcgctttttatagggccgccgccgccgccgcctcgccataaaaggaaactttcggagcgcgccgctctgattggctgccgccgcacctctccgcctcgccccgccccgcccctcgcccccatcgctgcacaaaataattaaaaaataaataaatacaaaattgggggtggggaggggggggagatggggagagtgaagcagaacgtggcctcggatcccccgggctgcagtattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagctcgctagcactagtggagcaccatggagctgtgcgggctggggctgccccggccgcccatgctgctggcgctgctgttggcgacactgctggcggcgatgttggcgctgctgactcaggtggcgctggtggtgcaggtggcggaggcggctcgggccccgagcgtctcggccaagccggggccggcgctgtggcccctgccgctctcggtgaagatgaccccgaacctgctgcatctcgccccggagaacttctacatcagccacagccccaattccacggcgggcccctcctgcaccctgctggaggaagcgtttcgacgatatcatggctatatttttggtttctacaagtggcatcatgaacctgctgaattccaggctaaaacccaggttcagcaacttcttgtctcaatcacccttcagtcagagtgtgatgctttccccaacatatcttcagatgagtcttatactttacttgtgaaagaaccagtggctgtccttaaggccaacagagtttggggagcattacgaggtttagagacctttagccagttagtttatcaagattcttatggaactttcaccatcaatgaatccaccattattgattctccaaggttttctcacagaggaattttgattgatacatccagacattatctgccagttaagattattcttaaaactctggatgccatggcttttaataagtttaatgttcttcactggcacatagttgatgaccagtctttcccatatcagagcatcacttttcctgagttaagcaataaaggaagctattctttgtctcatgtttatacaccaaatgatgtccgtatggtgattgaatatgccagattacgaggaattcgagtcctgccagaatttgatacccctgggcatacactatcttggggaaaaggtcagaaagacctcctgactccatgttacagtagacaaaacaagttggactcttttggacctataaaccctactctgaatacaacatacagcttccttactacatttttcaaagaaattagtgaggtgtttccagatcaattcattcatttgggaggagatgaagtggaatttaaatgttgggaatcaaatccaaaaattcaagatttcatgaggcaaaaaggctttggcacagattttaagaaactagaatctttctacattcaaaaggttttggatattattgcaaccataaacaagggatccattgtctggcaggaggtttttgatgataaagcaaagcttgcgccgggcacaatagttgaagtatggaaagacagcgcatatcctgaggaactcagtagagtcacagcatctggcttccctgtaatcctttctgctccttggtacttagatttgattagctatggacaagattggaggaaatactataaagtggaacctcttgattttggcggtactcagaaacagaaacaacttttcattggtggagaagcttgtctatggggagaatatgtggatgcaactaacctcactccaagattatggcctcgggcaagtgctgttggtgagagactctggagttccaaagatgtcagagatatggatgacgcctatgacagactgacaaggcaccgctgcaggatggtcgaacgtggaatagctgcacaacctctttatgctggatattgtaaccatgagaacatgtaactcgaggcggccgctctagagatctttttccctctgccaaaaattatggggacatcatgaagccccttgagcatctgacttctggctaataaaggaaatttattttcattgcaatagtgtgttggaattttttgtgtctctcactcggcatgctggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccatgcagccagctggcgtaatagcgaagaggcccgcaccgatcgcccttcccaacagttgcgtagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgggctgcaggggggggggggggggggtgggggggggggggggggg

SEQ ID NO: 8 - DNA, 7745 bp, synthetic construct
  misc_feature 1..7745: note = Synthetic polynucleotide
  source 1..7745: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattctaccacatttgtagaggttttacttgctttaaaaaacctcccacatctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtcgaggcggccgctcaggtctgctcaaattcctgctcacagaatccgacattcaggggctgtgcctgcacgcctctcctcagcagctcgcacctgaagtggctcagcctttcgtaagcgaatgtcagatcagaggtcagcttgttggaccacagtctctcggccactgctccggccctgggccacagtcgtgggaccagattggtgttgtccacatactcgccccacatacatgcttcgcccccgatgaccagagctttctgctcaggagtcccttcaaaggccaggggctccacaatgtagaaatccttccagtctggtccatatgagatcctgttcaggtaccaaggggcactcagcagtgctcggaagccagcttttgtcaccagttccagctccttcatataattgacaggaatatcctcgcgccacacttgaatgatggtatcgggctggattttgaccttgttgtcaaacacttcctgccagaccacatagccctttccgtatgaactcacaatgtccagcagagtctggatgtagaaactctccagctgcttgaagtcttcgccgaaccctttcttccgcataaagtcctggatctctggattagacttccaacatgtgaaatccacttcgtctccgcccaggtgcagataaaagtcagggaagacagaggacacctccagaaagaatgtagacatgaactcgtaggtattgttcagggatgggttcacaggtccgaaggtgccgcttggctcactcccagagtagcaaggagtcagcagtccgggaattcctgggccccagctcagagtatggccaggtgtatcgaattcagccagcactcgaatccccctcagtcgggcatactcgatgacttctttcacgtcctgggcggtatagatgtgagtcacgggattgtagctgcccttgcgcatcagctctggaaatgtgaagctttcgtaaggaaatgagggatcgtcgaccagatgccagtggaacacgttcagcttgttataagccatcacatccagagtgtccagaatgcttgacaggggcaggtaatgtctagatgtatccagcagcagcccccggtgtggaaatctagggaagtcttcgatctcagttttgttgataaagaatgttccctcggctgacttccagaccagctgactgaaagtctccaggcccctcagtgccccccacactgtttcggacagcagcagacactgatcgtcattgattgtcagggtatagttctccacggattccagggtgggcagctgattgcagcctggagtgaccacgctgaccaccagcacgtttttctccagggtgtggcgcttcccagtcagatagggccgtggccaggacccgcttccaaacagcagatcgcggtaccgctggaatgcctcgtccaggacgctgcatcctggctgagcggcgctgctcacgtcgtactgaaactggaaattgtttggatacagcacgtacctctggtcagatgtctggaagttctgaggccatggccacagggcggttgctcgtccagcaaatgcagcggccagcagcagggagaaccacagtctggagctggtcatggtggctttatcgatactagcgctagagcttgctcccgcccgccgcgcgcttcgctttttatagggccgccgccgccgccgcctcgccataaaaggaaactttcggagcgcgccgctctgattggctgccgccgcacctctccgcctcgccccgccccgcccctcgcccccatcgctgcacaaaataattaaaaaataaataaatacaaaattgggggtggggaggggggggagatggggagagtgaagcagaacgtggcctcggatcccccgggctgcagtattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagctcgctagcactagtaaagccaccatggaactgtgcggactgggactgcctagaccccctatgctgctggctctgctgctggctaccctgctggctgctatgctggccctgctgactcaggtggccctggtggtccaggtcgcagaggcagctagggctccttcagtgagcgcaaagccaggacctgccctgtggccactgccactgtccgtgaaaatgacaccaaacctgctgcacctggcccccgaaaatttctacatctcccattctcctaacagtacagcagggccatcatgcactctgctggaggaagccttccggagataccacgggtatattttcggcttctacaagtggcaccatgagcccgcagaatttcaggccaaaacccaggtgcagcagctgctggtcagcatcacactgcagtccgagtgtgacgctttcccaaatattagctccgatgagagctacaccctgctggtgaaggaacccgtggctgtcctgaaagcaaacagagtctggggcgcactgagggggctggagacattctcccagctggtgtaccaggactcttacggaacatttactatcaacgaatccactatcatcgactcccctaggttttctcaccgcggcatcctgattgatacttctcgccattacctgccagtgaagatcattctgaaaaccctggatgcaatggccttcaacaagtttaatgtgctgcactggcatatcgtcgacgatcagtctttcccttaccagagtattacatttccagagctgagtaataaaggcagttactcactgagccacgtgtatactccaaacgacgtgagaatggtcatcgagtatgccaggctgcgcggaattagggtgctgccagaattcgatactcccggccataccctgtcttggggcaaggggcagaaagacctgctgaccccctgctacagtcggcagaacaagctggattcattcggccctatcaacccaacactgaataccacatactcttttctgactaccttctttaaggagatcagtgaagtgttccccgaccagtttattcacctgggcggggatgaggtcgaattcaagtgttgggagagcaatcctaaaatccaggactttatgagacagaagggattcggcaccgacttcaagaaactggaatcattctacattcagaaggtgctggacatcattgccacaatcaacaaagggagcatcgtgtggcaggaggtctttgacgataaggccaaactggctcccggaactatcgtggaagtctggaaggatagcgcctatcctgaggaactgtcacgcgtgaccgctagcggctttcccgtcatcctgagcgcaccttggtacctggacctgatttcctatgggcaggattggcgaaagtactataaagtggagcccctggacttcggaggcacccagaagcagaaacagctgtttatcgggggagaggcctgcctgtggggggaatacgtggatgctaccaatctgacaccacgactgtggcctcgggctagcgcagtgggagagcgcctgtggtctagtaaggacgtgcgggatatggacgatgcctatgaccggctgacccgacatcggtgtagaatggtggaacggggcattgccgctcagccactgtatgccgggtactgtaatcacgaaaatatgtgactcgaggcggccgctctagagatctttttccctctgccaaaaattatggggacatcatgaagccccttgagcatctgacttctggctaataaaggaaatttattttcattgcaatagtgtgttggaattttttgtgtctctcactcggcatgctggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccatgcagccagctggcgtaatagcgaagaggcccgcaccgatcgcccttcccaacagttgcgtagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgggctgcaggggggggggggggggggtgggggggggggggggggg

SEQ ID NO: 9 - DNA, 7729 bp, synthetic construct
  misc_feature 1..7729: note = Synthetic polynucleotide
  source 1..7729: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattctaccacatttgtagaggttttacttgctttaaaaaacctcccacatctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtcgagtcaggtctgttcaaactcctgctcacagaagcctacattgaggggttgggcctggacacctcgcctcagcaactcacagcggaagtgtgacaaacgttcataggcaaatgtcaggtcagatgtcaacttgttgctccacagcctttcggcaacagcccctgctctgggccagagcctggggaccaggtttgtgttgtccacatattctccccacatacaagcctctccaccaatcaccagagccttctgctcaggggtaccttcaaatgccaggggttccactatgtagaaatccttccagtcagggccataggatatacggttcaggtaccagggggcagagagaagggcccggaagccggccttggtgaccagttccagctccttcatatagttcactggaatatcctctcgccacacctgtatgattgtgtctggctgaatctttactttattatcaaacacctcctgccacaccacatagcccttgccataagaagagacgatgtccagcagcgtctggatgtagaaggactccagctgcttgaagtcctcaccgaagcctttcttcctcataaagtcctggatctctgggttggacttccagcaggtgaaatcaacctcatctcctccaagatgaagataaaaatctgggaagacagagctgacttctaagaagaatgtgctcatgaactcataggtattattgagactgggattcactggtccaaaggtgccagagggctcagacccagagtagcaaggagtcagtaatccagggatacctggtccccaggacaaagtgtggccaggagtgtcaaactctgcaagcacacggataccccggagccgtgcgtattcaatgacctccttcacatcctgtgctgtgtagatgtgggtgacagggttgtaggacccctttctcatgagctctggaaaagtgaagctctcatatgggaaggaaggatcatctaccagatgccagtggaacacgttcaatttattgtacgccatgacatccagagtgtccaggatgctagagagtggcaggtaatggcgagatgtatccaacagcaagccccggtgaggaaagcggggaaagtcctcaatctcagtcttgttgataaagaatgtgccctcagcagatttccaaacaagctggctaaaagtctccagacctcggagagctccccagacagtctcagagaggagtaaacactggtcatcatttatggtcagggtataattctccactgactccaaagtaggaagctggttacatccaggtgtgactacagagacaaccaacacattcttctccagtgtatgccgtttccctgtgaggtaaggacggggccaagacccggaaccgaaaagcaggtcacgatagcgctggaaggcctcgtcgaggactgagcagccgggctgcgcggccgagctgacatcgtactggaattgaaagttgttcgggtaaaggacgtagcgctggtcggaggtttggaagttctgaggccagggccagagggccgtcgcccgtcctgcgaacgctgccgccagcagcagcgaaaaccaaagcctggaacttgtcatggtgctccactagcgctagagcttgctcccgcccgccgcgcgcttcgctttttatagggccgccgccgccgccgcctcgccataaaaggaaactttcggagcgcgccgctctgattggctgccgccgcacctctccgcctcgccccgccccgcccctcgcccccatcgctgcacaaaataattaaaaaataaataaatacaaaattgggggtggggaggggggggagatggggagagtgaagcagaacgtggcctcggatcccccgggctgcagtattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagctcgctagcactagtggagcaccatggagctgtgcgggctggggctgccccggccgcccatgctgctggcgctgctgttggcgacactgctggcggcgatgttggcgctgctgactcaggtggcgctggtggtgcaggtggcggaggcggctcgggccccgagcgtctcggccaagccggggccggcgctgtggcccctgccgctctcggtgaagatgaccccgaacctgctgcatctcgccccggagaacttctacatcagccacagccccaattccacggcgggcccctcctgcaccctgctggaggaagcgtttcgacgatatcatggctatatttttggtttctacaagtggcatcatgaacctgctgaattccaggctaaaacccaggttcagcaacttcttgtctcaatcacccttcagtcagagtgtgatgctttccccaacatatcttcagatgagtcttatactttacttgtgaaagaaccagtggctgtccttaaggccaacagagtttggggagcattacgaggtttagagacctttagccagttagtttatcaagattcttatggaactttcaccatcaatgaatccaccattattgattctccaaggttttctcacagaggaattttgattgatacatccagacattatctgccagttaagattattcttaaaactctggatgccatggcttttaataagtttaatgttcttcactggcacatagttgatgaccagtctttcccatatcagagcatcacttttcctgagttaagcaataaaggaagctattctttgtctcatgtttatacaccaaatgatgtccgtatggtgattgaatatgccagattacgaggaattcgagtcctgccagaatttgatacccctgggcatacactatcttggggaaaaggtcagaaagacctcctgactccatgttacagtagacaaaacaagttggactcttttggacctataaaccctactctgaatacaacatacagcttccttactacatttttcaaagaaattagtgaggtgtttccagatcaattcattcatttgggaggagatgaagtggaatttaaatgttgggaatcaaatccaaaaattcaagatttcatgaggcaaaaaggctttggcacagattttaagaaactagaatctttctacattcaaaaggttttggatattattgcaaccataaacaagggatccattgtctggcaggaggtttttgatgataaagcaaagcttgcgccgggcacaatagttgaagtatggaaagacagcgcatatcctgaggaactcagtagagtcacagcatctggcttccctgtaatcctttctgctccttggtacttagatttgattagctatggacaagattggaggaaatactataaagtggaacctcttgattttggcggtactcagaaacagaaacaacttttcattggtggagaagcttgtctatggggagaatatgtggatgcaactaacctcactccaagattatggcctcgggcaagtgctgttggtgagagactctggagttccaaagatgtcagagatatggatgacgcctatgacagactgacaaggcaccgctgcaggatggtcgaacgtggaatagctgcacaacctctttatgctggatattgtaaccatgagaacatgtaactcgaggcggccgctctagagatctttttccctctgccaaaaattatggggacatcatgaagccccttgagcatctgacttctggctaataaaggaaatttattttcattgcaatagtgtgttggaattttttgtgtctctcactcggcatgctggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccatgcagccagctggcgtaatagcgaagaggcccgcaccgatcgcccttcccaacagttgcgtagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgggctgcaggggggggggggggggggtgggggggggggggggggg